SEQUENCE LISTING (57 sequences)

SEQ ID NO: 1 - protein, 317 aa, synthetic construct
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 2 - protein, 10 aa, synthetic construct
GVYDGREHTV

SEQ ID NO: 3 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 4 - DNA, 745 bp, synthetic construct
  source 1..745: mol_type = other DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 5 - protein, 282 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 6 - DNA, 846 bp, synthetic construct
  source 1..846: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 7 - protein, 311 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 8 - DNA, 933 bp, synthetic construct
  source 1..933: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 9 - protein, 136 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 10 - protein, 133 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 11 - protein, 6 aa, synthetic construct
VSPFSN

SEQ ID NO: 12 - protein, 6 aa, synthetic construct
LTFSEN

SEQ ID NO: 13 - protein, 15 aa, synthetic construct
CVVSGGTDSWGKLQF

SEQ ID NO: 14 - protein, 5 aa, synthetic construct
KGHDR

SEQ ID NO: 15 - protein, 6 aa, synthetic construct
SFDVKD

SEQ ID NO: 16 - protein, 14 aa, synthetic construct
CATSGQGAYEEQFF

SEQ ID NO: 17 - protein, 9 aa, synthetic construct
VLLSNPTSG

SEQ ID NO: 18 - protein, 8 aa, synthetic construct
YLSQNKPK

SEQ ID NO: 19 - protein, 6 aa, unidentified
LSNSIM

SEQ ID NO: 20 - protein, 609 aa, synthetic construct
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 21 - protein, 9 aa, synthetic construct
FMNKFIYEI

SEQ ID NO: 22 - protein, 205 aa, synthetic construct
QKEVEQNSGPLSVPEGAIASLNCTYSDRGSQSFFWYRQYSGKSPELIMSIYSNGDKEDGRFTAQLNKASQYVSLLIRDSQPSDSATYLCAVNSDSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 23 - protein, 242 aa, synthetic construct
DSGVTQTPKHLITATGQRVTLRCSPRSGDLSVYWYQQSLDQGLQFLIQYYNGEERAKGNILERFSAQQFPDLHSELNLSSLELGDSALYFCASSLGGESEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 24 - DNA, 615 bp, synthetic construct
  source 1..615: mol_type = other DNA
caaaaagaagttgagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaatagtgattccgggtatgcactcaacttcggcaaaggcacctcgctgttggtcacaccccatatccagaaccctgaccctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcc

SEQ ID NO: 25 - DNA, 726 bp, synthetic construct
  source 1..726: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcctcgggggggaatctgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 26 - protein, 6 aa, synthetic construct
DRGSQS

SEQ ID NO: 27 - protein, 6 aa, synthetic construct
IYSNGD

SEQ ID NO: 28 - protein, 12 aa, synthetic construct
AVNSDSGYALNF

SEQ ID NO: 29 - protein, 5 aa, synthetic construct
SGDLS

SEQ ID NO: 30 - protein, 6 aa, synthetic construct
YYNGEE

SEQ ID NO: 31 - protein, 11 aa, synthetic construct
ASSLGGESEQY

SEQ ID NO: 32 - protein, 6 aa, synthetic construct
DRGSQA

SEQ ID NO: 33 - protein, 12 aa, synthetic construct
AVNSDSSYALNF

SEQ ID NO: 34 - protein, 12 aa, synthetic construct
AVNSDSGVALNF

SEQ ID NO: 35 - protein, 12 aa, synthetic construct
AVNSQSGYALNF

SEQ ID NO: 36 - protein, 12 aa, synthetic construct
AVNSQSGYSLNF

SEQ ID NO: 37 - protein, 12 aa, synthetic construct
AVNSQNGYALNF

SEQ ID NO: 38 - protein, 6 aa, synthetic construct
DRGSFS

SEQ ID NO: 39 - protein, 6 aa, synthetic construct
DRGSYS

SEQ ID NO: 40 - protein, 12 aa, synthetic construct
AVNSQSSYALNF

SEQ ID NO: 41 - protein, 205 aa, synthetic construct
QKEVEQNSGPLSVPEGAIASLNCTYSDRGSQAFFWYRQYSGKSPELIMSIYSNGDKEDGRFTAQLNKASQYVSLLIRDSQPSDSATYLCAVNSQSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 42 - protein, 242 aa, synthetic construct; identical to SEQ ID NO: 23

SEQ ID NO: 43 - DNA, 855 bp, synthetic construct
  source 1..855: mol_type = other DNA
agttgatagagtactgtctgccttcataggtatttagtatgctgtaaatatttttaggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtaacaatttaaagtaacattaacttattgcagaggctaaagtcatttgagactttggatttgcactgaatgcaaatcttttttccaaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtaaggatgactacttaaatgtaaaaaagttgtgttaaagatgaaaaatacaactgaacagtactttgggtaataattaactttttttttaataggtcgctttgctgttcgtgatatgagacagacagttgcggtgggtgtcatcaaagcagtggacaagaaggctgctggagctggcaaggtcaccaagtctgcccagaaagctcagaaggctaaa

SEQ ID NO: 44 - DNA, 949 bp, synthetic construct
  source 1..949: mol_type = other DNA
tgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgtgttgcatagaaagcttcatgttgctaaaccaatgttaagtgaatctttggaaacaaaatgtttccaaattactgggatgtgcatgttgaaacgtgggttaaaatgactgggcagtgaaagttgactatttgccatgacataagaaataagtgtagtggctagtgtacaccctatgagtggaagggtccattttgaagtcagtggagtaagctttatgccagtttgatggtttcacaagttctattgagtgctattcagaataggaacaaggttctaatagaaaaagatggcaatttgaagtagctataaaattagactaatctacattgcttttctcctgcagagtctaataccttttatgctttgataattagcagtttgtctac

SEQ ID NO: 45 - DNA, 1944 bp, synthetic construct
  source 1..1944: mol_type = other DNA
tccagcggcagccgggccaagagatctggatcaggtgagggcagaggcagcctgctgacatgtggcgacgtggaagaaaaccctggccctatgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagagatctggatccggcgctaccaactttagcctgctgaagcaggccggggacgtggaagaaaaccctggccctaggatggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggctaa

SEQ ID NO: 46 - DNA, 49 bp, synthetic construct
  source 1..49: mol_type = other DNA
aataaaatatctttattttcattacatctgtgtgttggttttttgtgtg

SEQ ID NO: 47 - DNA, 230 bp, synthetic construct
  source 1..230: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 48 - DNA, 34 bp, synthetic construct
  source 1..34: mol_type = other DNA
ataacttcgtataatgtatgctatacgaagttat

SEQ ID NO: 49 - DNA, 597 bp, synthetic construct
  source 1..597: mol_type = other DNA
atgaccgagtacaagcccaccgtgaggctggctaccagagacgacgtgcccagagccgtgagaaccctggctgccgccttcgctgactaccctgccaccaggcacaccgtggatcccgacaggcacatcgagagggtgaccgagctgcaggagctgttcctgaccagggtgggcctggacatcggcaaggtgtgggtggctgacgatggagccgctgtggccgtgtggacaacccccgagagcgtggaagctggagccgtgttcgccgagatcggccctagaatggccgagctgtccggcagcaggctggctgctcagcagcagatggagggcctgctggcccctcacagacctaaggagcccgcctggttcctggccaccgtgggagtgagccctgaccaccagggcaagggactgggctccgctgtggtgctgcctggagtggaagccgctgagagagccggagtgcccgcctttctggaaacctccgcccccaggaacctgcccttctacgagaggctgggcttcaccgtgaccgccgatgtggaagtgcctgagggccccaggacctggtgcatgaccaggaagcccggcgcc

SEQ ID NO: 50 - DNA, 25 bp, synthetic construct
  source 1..25: mol_type = other DNA
taagttggctgtaaacaaagttgaa

SEQ ID NO: 51 - DNA, 18 bp, synthetic construct
  source 1..18: mol_type = other DNA
gcacgtcgtctcggtagc

SEQ ID NO: 52 - DNA, 21 bp, synthetic construct
  source 1..21: mol_type = other DNA
tctacgagaggcctgggcttc

SEQ ID NO: 53 - DNA, 20 bp, synthetic construct
  source 1..20: mol_type = other DNA
gtggggtggcaggtattagg

SEQ ID NO: 54 - DNA, 21 bp, synthetic construct
  source 1..21: mol_type = other DNA
ggaacccctagtgatggagtt

SEQ ID NO: 55 - DNA, 16 bp, synthetic construct
  source 1..16: mol_type = other DNA
cggcctcagtgagcga

SEQ ID NO: 56 - DNA, 663 bp, synthetic construct
  source 1..663: mol_type = other DNA
ggaagcggagctactaacttcagcctgctgaagcaggctggagacgtggaggagaaccctggacctatgaccgagtacaagcccaccgtgaggctggctaccagagacgacgtgcccagagccgtgagaaccctggctgccgccttcgctgactaccctgccaccaggcacaccgtggatcccgacaggcacatcgagagggtgaccgagctgcaggagctgttcctgaccagggtgggcctggacatcggcaaggtgtgggtggctgacgatggagccgctgtggccgtgtggacaacccccgagagcgtggaagctggagccgtgttcgccgagatcggccctagaatggccgagctgtccggcagcaggctggctgctcagcagcagatggagggcctgctggcccctcacagacctaaggagcccgcctggttcctggccaccgtgggagtgagccctgaccaccagggcaagggactgggctccgctgtggtgctgcctggagtggaagccgctgagagagccggagtgcccgcctttctggaaacctccgcccccaggaacctgcccttctacgagaggctgggcttcaccgtgaccgccgatgtggaagtgcctgagggccccaggacctggtgcatgaccaggaagcccggcgcc

SEQ ID NO: 57 - DNA, 2577 bp, synthetic construct
  source 1..2577: mol_type = other DNA
ggaagcggagctactaacttcagcctgctgaagcaggctggagacgtggaggagaaccctggacctatgaccgagtacaagcccaccgtgaggctggctaccagagacgacgtgcccagagccgtgagaaccctggctgccgccttcgctgactaccctgccaccaggcacaccgtggatcccgacaggcacatcgagagggtgaccgagctgcaggagctgttcctgaccagggtgggcctggacatcggcaaggtgtgggtggctgacgatggagccgctgtggccgtgtggacaacccccgagagcgtggaagctggagccgtgttcgccgagatcggccctagaatggccgagctgtccggcagcaggctggctgctcagcagcagatggagggcctgctggcccctcacagacctaaggagcccgcctggttcctggccaccgtgggagtgagccctgaccaccagggcaagggactgggctccgctgtggtgctgcctggagtggaagccgctgagagagccggagtgcccgcctttctggaaacctccgcccccaggaacctgcccttctacgagaggctgggcttcaccgtgaccgccgatgtggaagtgcctgagggccccaggacctggtgcatgaccaggaagcccggcgccggatcgggagagggcagaggaagtctgctaacatgcggtgacgtcgaggagaatcctggcccaatgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagagatctggatccggcgctaccaactttagcctgctgaagcaggccggggacgtggaagaaaaccctggccctaggatggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

(19) United States
(12) Patent Application Publication
(10) Pub. No.: US 2024/0392244 A1
(43) Pub. Date: Nov. 28, 2024

(54) MODIFICATION OF CELLS WITH HETEROLOGOUS T-CELL RECEPTOR (TCR) CODING SEQUENCES

(71) Applicant: ADAPTIMMUNE LIMITED, Abingdon, Oxfordshire (GB)

(72) Inventor: Garth HAMILTON, Abingdon, Oxfordshire (GB)

(21) Appl. No.: 18/291,895

(22) PCT Filed: Aug. 3, 2022
(86) PCT No.: PCT/GB2022/052048

§ 371 Date: Jan. 24, 2024

(30) Foreign Application Priority Data
Aug. 3, 2021 (GB) 2111214.9

(51) Int. Cl. C12N 5/0783; A61K 39/00; C07K 14/725

(52) U.S. Cl. CPC C12N 5/0636; A61K 39/4611; A61K 39/4632; A61K 39/464486; C07K 14/7051; C12N 2501/115; C12N 2501/125; C12N 2501/155; C12N 2501/16; C12N 2501/165; C12N 2501/727; C12N 2506/45; C12N 2510/00

(57) ABSTRACT

The present invention provides a modified iPSC or haemogenic lineage cell, such as a T cell, comprising at least one heterologous nucleic acid sequence encoding a heterologous T-cell receptor (TCR) integrated in the cell genome, for example in the eukaryotic translation elongation factor 1 alpha 1 (EEF1A1) gene and uses thereof.

### FIELD

The present invention provides a modified induced pluripotent stem cell (iPSC) or haemogenic lineage cell comprising at least one heterologous nucleic acid sequence encoding a heterologous T-cell receptor (TCR) integrated in the cell genome and uses thereof.

### BACKGROUND

Adoptive T-cell therapy is widely recognized as an important therapeutic intervention for the treatment of cancer. Most current approaches use autologous or patient-derived T-cells. These include TILs (tumour infiltrating lymphocytes) or T-cells that have been virally transduced to express a CAR (chimeric antigen receptor) or affinity enhanced TCR (T-cell receptor).

One drawback of autologous therapies is the complexity associated with their manufacture. An alternative “off-the-shelf” approach is the production of engineered T-cells from an allogeneic source, such healthy donor derived T-cells or T-cells that have been differentiated from a human induced pluripotent stem cell (hiPSC). Additionally, patients must wait for an apheresis slot to collect their own lymphocytes and the manufacturing time for the T cell product to be made, additionally patients may also suffer T cell dysfunction or have had multiple lines of prior chemotherapy, impairing their own lymphocyte count and function such that the cells available for manufacture are low in count or functionality at the outset.

The allogeneic approach is particularly advantageous as it may provide a more readily available supply of a uniform cell product that can be screened for a T cell phenotype linked with more a more functional and potent, anti-cancer capability associated with improved response. In short patients will get treatment faster with a more defined T-cell phenotype that does not vary from patient to patient. It is expected that gene editing can provide quick access to T cell therapy at reduced cost for a standardized high quality T cell product, for a much wider group of patients who have the most aggressive and resistant disease.

A prerequisite for any allogeneic T-cell product which uses an αβTCR is the production of a clonal T-cell population. This is required to mitigate the risk of graft versus host disease (GvHD). T cells derived from iPSCs are commonly referred to as iT cells. iT-cells with restricted expression of a defined αβTCR have been successfully produced from clones of hiPSC that were transduced with lentiviral vectors encoding a specific TCR [Minagawa, A., et al., Enhancing T Cell Receptor Stability in Rejuvenated iPSC-Derived T Cells Improves Their Use in Cancer Immunotherapy. Cell Stem Cell, 2018. 23 (6): p. 850-858 e4]. Related studies have suggested that restriction of αβTCR expression in differentiated iT-cells is driven by TCR allelic exclusion, which prevents erroneous rearrangement of endogenous TCR genes. However, due to the phenomenon of promoter silencing, lentiviral transduction is an inefficient method at promoting stable transgene expression in hiPSC and differentiated cells [Zou, J., et al., Oxidase-deficient neutrophils from X-linked chronic granulomatous disease iPS cells: functional correction by zinc finger nuclease-mediated safe harbor targeting. Blood, 2011. 117 (21): p. 5561-72]. In addition, the development of a TiPSC derived from a recombinant heterologous T-cell receptor expressing T-cell would require additional interventions to prevent expression of the native TCR present in the initial T-cell material.

An alternative approach is to genetically engineer the hiPSC to prevent endogenous TCR expression in differentiated iT-cells. This is a challenging approach as it requires the inactivation of multiple genes in both alleles, for example TCR constant domains (TRBC1/2 and TRAC) or possibly genes involved in TCR gene rearrangement (e.g RAG1/2) in conjunction with the heterologous T-cell receptor knock-in. It is however difficult and unpredictable to ascertain what loci to target that will permit TCR expression in differentiated iT-cells.

### SUMMARY

The present inventors have demonstrated that targeted insertion of a recombinant heterologous T-cell receptor sequence permits expression in differentiated iT-cells using a minimal editing strategy. Aspects of the invention are provided in the following numbered statements.
1. A modified iPSC or haemogenic lineage cell comprising at least one heterologous nucleic acid sequence encoding a heterologous T-cell receptor (TCR) integrated at or into a locus in the cell genome.

In preferred embodiments of the aspect of statement 1, the locus in the genome is a gene encoding a member of the elongation factor complex, preferably a translation elongation factor, preferably eukaryotic translation elongation factor 1 alpha 1 (EEF1A1).
2. The modified iPSC or haemogenic lineage cell according to statement 1, wherein the at least one heterologous nucleic acid sequence encoding a heterologous TCR is an expressible heterologous nucleic acid sequence.
3. The modified iPSC or haemogenic lineage cell according to either statement 1 or statement 2, wherein the modified iPSC or haemogenic lineage cell expresses or presents the at least one heterologous TCR encoded by a heterologous TCR encoding nucleic acid sequence, preferably expressed or presented at the cell surface.
4. The modified iPSC or haemogenic lineage cell according to any previous statement, wherein the modified haemogenic lineage cell is derived from the modified iPSC.
5. The modified iPSC or haemogenic lineage cell according to any previous statement, wherein the haemogenic lineage cell is selected from any one of;
   (a) a mesoderm cell, optionally which may express any one or more of the mesodermal markers, Brachyury, Goosecoid, MixI1, KDR (also known as FLK1 or VEGFR2), FoxA2, GATA6 or PDGF alpha R,
   (b) a haemogenic endothelial cell, optionally which may be (a) CD34+or (b) CD34+ CD73− or (c) CD34+ CD73− CXCR4− (CD184−),
   (c) a haematopoietic progenitor cell, optionally which may be (a) CD34+ or (b) CD34+ CD45+ or (c) CD34+ and/or CD45+ in combination with any one or more of CD117+, CD133+, CD45+, FLK+, CD38−, (d) CD34+, CD133+, CD45+, FLK1+, CD38−
   (d) a progenitor T cell, optionally which may be (a) CD5+ and/or CD7+, or (b) CD5+ and/or CD7+ in combination with any one or more of: CD44+, CD25+, CD2+, CD45+, CD3−, CD4−, CD8−,
   (e) a double positive T cell (DP T cell), optionally which may be (a) CD4+ CD8+ (b) CD4+ CD8+ in combination with any one or more of CD3+, CD28+, CD45+,
   (f) a single positive T cell (SP T cell) optionally which may be (a) CD4+ (b) CD8+ (c) CD4+ or CD8+ in combination with any one or more of CD3+, CD28+, CD45+, or
   (g) a mature T cell, optionally alpha beta T cell, gamma delta T cell, NKT cell, T helper cell (TH) which are CD4+, cytotoxic T cell (TC) which are CD8+.
6. The modified iPSC or haemogenic lineage cell according to any previous statement, wherein the heterologous nucleic acid sequence encoding a heterologous TCR is integrated at or into one or both alleles of the locus in the cell genome.
7. The modified iPSC or haemogenic lineage cell according to statement 6, wherein the locus is a gene encoding an endogenous protein of the cell.
8. The modified iPSC or haemogenic lineage cell according to statement 7, wherein the heterologous nucleic acid sequence encoding a heterologous TCR is integrated adjacent to or within the gene encoding the endogenous protein.
9. The modified iPSC or haemogenic lineage cell according to either of statements 7 or 8, wherein the heterologous nucleic acid sequence encoding a heterologous TCR is integrated within an intron or exon of the gene encoding the endogenous protein, optionally a 3′ exon.
10 The modified iPSC or haemogenic lineage cell according to statement 9, wherein the heterologous nucleic acid sequence encoding a heterologous TCR is integrated within the 3′ exon before the TAG stop sequence of the gene or nucleic acid sequence encoding the endogenous protein.
11. The modified iPSC or haemogenic lineage cell according to any one of statements 7to 10, wherein the integration of the heterologous nucleic acid sequence encoding a heterologous TCR is non-disruptive to the production of the endogenous protein.
12. The modified iPSC or haemogenic lineage cell according to any one of statements 7to 11, comprising a fusion sequence between the nucleic acid encoding the heterologous TCR and the nucleic acid encoding the endogenous gene, preferably a fusion gene or sequence or multicistronic fusion gene or sequence between the nucleic acid encoding the heterologous TCR and the nucleic acid encoding the endogenous protein.
13. The modified iPSC or haemogenic lineage cell according to statement 12 wherein the nucleic acid encoding the heterologous TCR is connected to the nucleic acid encoding the endogenous protein by a nucleic acid sequence encoding a peptide comprising an enzymatic cleavage site and/or a nucleic acid sequence which mediates ribosome-skipping.
14. The modified iPSC or haemogenic lineage cell according to either of statements 12 or 13, wherein the nucleic acid encoding the heterologous TCR comprises a coding sequence of a TCRα and TCRβ chain, optionally with an intervening nucleic acid sequence encoding a peptide comprising an enzymatic cleavage site and/or nucleic acid sequence which mediates ribosome-skipping.
15. The modified iPSC or haemogenic lineage cell according to either of statements 13 or 14, wherein the nucleic acid sequence which mediates ribosome-skipping is a T2A or P2A skip sequence.
16. The modified iPSC or haemogenic lineage cell according to any one of statements 13 to 15, wherein the nucleic acid sequence encoding a peptide comprising an enzymatic cleavage site encodes a furin cleavage site, preferably RAKR.
17. The modified iPSC or haemogenic lineage cell according to any one of statements 7 to 16, wherein the transcription and expression of the heterologous TCR and the endogenous protein is from the same promoter.
18. The modified iPSC or haemogenic lineage cell according to any one of statements 7 to 17, wherein the heterologous TCR is expressed as a fusion protein with the endogenous protein, optionally connected by a peptide comprising an enzymatic cleavage site, preferably a furin cleavage site, preferably RAKR and/or ribosome skip sequence, preferably T2A or P2A skip sequence.
19. The modified iPSC or haemogenic lineage cell according to any one of statements 1 to 18, wherein the heterologous TCR is expressed and/or presented in the cell as a nascent heterologous TCR comprising a TCR alpha chain and TCR beta chain, preferably expressed and/or presented at the cell surface.
20. The modified iPSC or haemogenic lineage cell according to any one of statements 7 to 19, wherein the endogenous protein is a member of the elongation factor complex, preferably a translation elongation factor, preferably eukaryotic translation elongation factor 1 alpha 1.
21. The modified iPSC or haemogenic lineage cell according to any one of statements 3 to 20, wherein the endogenous protein is a member of the elongation factor complex, preferably a translation elongation factor, preferably eukaryotic translation elongation factor 1 alpha 1.
22. The modified iPSC or haemogenic lineage cell according to any previous statement, wherein the locus is or is in a gene encoding a member of the elongation factor complex, preferably a translation elongation factor, preferably eukaryotic translation elongation factor 1 alpha 1 optionally wherein the locus is at exon 8 of the eukaryotic translation elongation factor 1 alpha 1 (EEF1A1) gene, optionally before the TAG stop codon or immediately adjacent to and/or before the TAG stop codon.
23. The modified iPSC or haemogenic lineage cell according to any previous statement, wherein the locus is or is in the EEF1A gene on chromosome 6.
24. The modified iPSC or haemogenic lineage cell according to statement 23, wherein the integration is at exon 8 of the EEF1A1 gene, optionally before the TAG stop codon or immediately adjacent to and/or before the TAG stop codon.
25. The modified iPSC or haemogenic lineage cell according to any one of statements 12 to 24, wherein the fusion gene or sequence or multicistronic fusion gene or sequence comprises the nucleic acid encoding the heterologous TCR and nucleic acid encoding EEF1A1 with an intervening nucleic acid sequence encoding a peptide comprising an enzymatic cleavage site, preferably a furin cleavage site, and nucleic acid sequence which mediates ribosome-skipping, preferably selected from a T2A or P2A skip sequence and wherein the nucleic acid sequence encoding the heterologous TCR comprises the coding sequence of a TCRα and TCRβ chain, with an intervening nucleic acid sequence encoding a peptide comprising an enzymatic cleavage site, preferably a furin cleavage site, and nucleic acid sequence which mediates ribosome-skipping, preferably selected from T2A or P2A skip sequence.
26. The modified iPSC or haemogenic lineage cell according to any previous statement, wherein the heterologous TCR binds or specifically binds to an antigen or peptide antigen thereof selected from,
   (a) a cancer and/or tumour antigen or peptide antigen thereof, or
   (b) a cancer and/or tumour antigen or peptide antigen thereof associated with a cancerous condition and/or presented by tumour or cancer cell or tissue.
27. The modified iPSC or haemogenic lineage cell according to statement 26 wherein the cancer and/or tumour antigen or peptide antigen thereof is selected from;
   (a) a cancer-testis antigen,
   (b) a MAGE antigen,
   (c) MAGE A4 or peptide antigen thereof, optionally a peptide antigen comprising the sequence GVYDGREHTV (SEQ ID NO: 2), or
   (d) AFP or peptide antigen thereof, optionally a peptide antigen comprising the sequence FMNKFIYEI (SEQ ID No: 21).
28. The modified iPSC or haemogenic lineage cell according to either one of statements 26 or 27 wherein the cancer and/or tumour antigen or peptide antigen thereof is complexed with a peptide presenting molecule, optionally major histocompatibility complex (MHC) or human leukocyte antigen (HLA), optionally class I or class II, optionally HLA-A2 or HLA-A*02, or HLA-A*0201.
29. The modified iPSC or haemogenic lineage cell according to any previous statement, wherein the heterologous TCR comprises a TCR having;
   (a) an alpha chain variable domain comprising an amino acid sequence that has at least 80%, identity to SEQ ID NO:9 or the sequence of amino acid residues 1-136 of SEQ ID NO: 6, and/or the beta chain variable domain comprising an amino acid sequence that has at least 80%, identity to SEQ ID NO: 10 or the sequence of amino acid residues 1-133 of SEQ ID NO:7, or
   (b) an alpha chain variable domain comprising an amino acid sequence that has at least 80%, identity to the sequence of amino acid residues 1-112 of SEQ ID NO:22, and/or the beta chain variable domain comprising an amino acid sequence that has at least 80%, identity to the sequence of amino acid residues 1-112 of SEQ ID NO:23.
30. The modified iPSC or haemogenic lineage cell according to any previous statement, wherein the iPSC or haemogenic lineage cell further comprises a nucleic acid encoding and/or expresses or presents a heterologous co-receptor, optionally wherein the co-receptor is a CD8 co-receptor.
31. The modified iPSC or haemogenic lineage cell according to statement 30, wherein the heterologous CD8 co-receptor is heterodimer or homodimer, a CD8αb heterodimer or a CD8αα homodimer.
32. The modified iPSC or haemogenic lineage cell according to either one of statements 30 or 31, wherein the heterologous CD8 co-receptor comprises;
   (a) a CDR 1 of at least 80% sequence identity to amino acid sequence VLLSNPTSG, SEQ ID NO:17, CDR 2 of at least 80% sequence identity to amino acid sequence YLSQNKPK SEQ ID NO: 18 and CDR 3 of at least 80% sequence identity amino acid sequence LSNSIM SEQ ID NO: 19, or
   (b) an amino acid sequence having at least 80% sequence identity to amino acids number 22 to 235 of SEQ ID NO: 19.
33. The modified iPSC or haemogenic lineage cell according to any previous statement, wherein the iPSC or haemogenic lineage cell further comprises a nucleic acid encoding and/or expresses or presents a heterologous co-stimulatory ligand; optionally 4-1BBL or CD80.
34. The modified iPSC or haemogenic lineage cell according to any one of statements 1 to 33, wherein the binding of the iPSC or haemogenic lineage cell and/or heterologous TCR to the cancer and/or tumour antigen or peptide antigen thereof or a cancer and/or tumour cell or tissue expressing or presenting the cancer and/or tumour antigen or peptide antigen thereof according to any of statements 26 to 28 induces activation of the iPSC or haemogenic lineage cell, optionally as determined by any one or more of;
   (a) up-regulation of T cell activation markers, for example either CD69 and/or CD25 on CD3+ cells,
   (b) up-regulation of cytokine production, for example any one or more of IFN gamma, IL-2 or Granzyme B,
   (c) induced cell cytotoxic activity in the presence of the antigen or antigen peptide, or
   (d) ability to kill tumour cells presenting the antigen or antigen peptide.
35. A nucleic acid construct or vector comprising a nucleic acid region encoding the heterologous TCR according to any previous statement and at least one homology region comprising a nucleic acid region homologous to a nucleic acid region at the locus in the cell genome for integration of the nucleic acid region encoding the heterologous TCR.
36. The nucleic acid construct or vector according to statement 35, wherein the locus is or is in a gene encoding a member of the elongation factor complex, preferably a translation elongation factor, preferably eukaryotic translation elongation factor 1 alpha 1 optionally wherein the locus is at exon 8 of the eukaryotic translation elongation factor 1 alpha 1 (EEF1A1) gene, optionally before the TAG stop codon or immediately adjacent to and/or before the TAG stop codon.
37. The nucleic acid construct or vector according to either statement 35 or 36, wherein the locus is or is in the EEF1A1 gene on chromosome 6.
38. The nucleic acid construct or vector according to any one of statements 35 to 37, wherein the locus is at exon 8 of the EEF1A1 gene, optionally before the TAG stop codon or immediately adjacent to and/or before the TAG stop codon.
39. The nucleic acid construct or vector according to any one of statements 35 to 38, wherein the nucleic acid encoding the heterologous TCR comprises a coding sequence of a TCRα and TCRβ chain, optionally with an intervening nucleic acid sequence encoding a peptide comprising an enzymatic cleavage site and/or nucleic acid sequence which mediates ribosome-skipping, preferably a furin cleavage site, preferably RAKR and/or ribosome skip sequence, preferably T2A or P2A skip sequence.
40. The nucleic acid construct or vector according to any one of statements 35 to 39, wherein the construct or vector comprises a left hand and a right hand homology region each homologous to a nucleic acid region at the locus in the cell genome for integration of the nucleic acid region encoding the heterologous TCR and which flank opposite sides of the integration site.
41. The nucleic acid construct or vector according to any one of statements 35 to 40, wherein the construct or vector further comprises any one or more of:
   (a) a recombination target sequence, preferably loxP (locus of X-over P1) sequence, such as the sequence of SEQ ID NO: 48,
   (b) an expressible selection marker sequence, preferably an antibiotic resistance gene, and optionally a neomycin resistance gene, preferably constitutively expressed from a promoter for example from an EF1A promoter such as the sequence of SEQ ID NO: 47.
42. The nucleic acid construct or vector according to any one of statements 35 to 41, wherein the construct or vector comprises a nucleotide sequence encoding the heterologous TCR comprising the sequence SEQ ID No: 45, homology regions comprising the sequences SEQ ID No: 43 and SEQ ID No: 44, a recombination target sequence comprising SEQ ID No: 48 and an expressible selection marker sequence comprising SEQ ID No: 47 and SEQ ID No: 49.
43. A process of producing a modified iPSC or haemogenic lineage cell according to any one of statements 1 to 34 comprising introducing the nucleic acid construct or vector according to any one of statements 35 to 42 into an unmodified iPSC or haemogenic lineage cell, optionally as defined in statement 5, under conditions to permit integration of the nucleic acid sequence encoding a heterologous T-cell receptor (TCR) at or into a locus in the cell genome and optionally isolating the modified iPSC or haemogenic lineage cell.
44. A pharmaceutical composition comprising the modified iPSC or haemogenic lineage cell according to any one of statements 1 to 34 and a pharmaceutically acceptable carrier.
45. The modified iPSC or haemogenic lineage cell according to any one of statements 1 to 34, or pharmaceutical composition of statement 44, for use in therapy and/or medicine.
46. The modified iPSC or haemogenic lineage cell according to any one of statements 1 to 34, or pharmaceutical composition of statement 44, for use in treatment, prevention or delaying the progression of cancer and/or tumour in an individual or subject optionally wherein the treatment is cancer immunotherapy therapy and/or adoptive T cell therapy, optionally allogenic adoptive T cell therapy.
45. Use of the modified iPSC or haemogenic lineage cell according to any one of statements 1 to 34, or pharmaceutical composition of statement 44, in the manufacture of a medicament for the treatment of cancer and/or tumour in an individual or subject, optionally wherein the treatment is cancer immunotherapy therapy and/or adoptive T cell therapy, optionally allogenic adoptive T cell therapy.
46. A method of treating, preventing or delaying the progression of cancer and/or tumour in an individual or subject comprising administering to the individual the modified iPSC or haemogenic lineage cell according to any one of statements 1 to 34, or pharmaceutical composition of statement 44, optionally wherein the treatment is cancer immunotherapy therapy and/or adoptive T cell therapy, optionally allogenic adoptive T cell therapy.
47. The modified iPSC or haemogenic lineage cell according to any one of statements 1 to 34, or pharmaceutical composition of statement 44, for use according to statement 45 or in the method of statement 46, wherein the cancer and/or tumour is a solid tumour.
48. The modified iPSC or haemogenic lineage cell according to any one of statements 1 to 34, or pharmaceutical composition of statement 44, for use according to statement 45, or in the method of statement 46, wherein the modified iPSC or haemogenic lineage cell, or pharmaceutical composition is for use or used in combination with one or more further therapeutic agent optionally administered or for administration separately, sequentially or simultaneously.
49. A kit comprising,
   (a) the modified iPSC or haemogenic lineage cell according to any one of statements 1 to 34, or pharmaceutical composition of statement 44, and a package insert comprising instructions for use thereof for treating, preventing or delaying the progression of cancer and/or tumour in an individual or subject, or
   (b) the modified iPSC or haemogenic lineage cell according to any one of statements 1 to 34, or pharmaceutical composition of statement 44, and a package insert comprising instructions for use thereof for treating, preventing or delaying the progression of cancer and/or tumour in an individual or subject, in combination with one or more further therapeutic agent optionally administered or for administration separately, sequentially or simultaneously.

In some preferred embodiments of the aspects of any one of statements 1 to 49, the modified iPSC or haemogenic lineage cell is a T cell, for example a DP T cell, a SP T cell or a mature T cell.
50. A method of producing a population of T cells comprising;
   (i) introducing a heterologous nucleic acid encoding T-cell receptor at or into a locus in the cell genome of a population of iPSCs, and
   (iii) differentiating the population of iPSCs into T cells.

In preferred embodiments of the aspect of statement 50, the locus in the genome is a gene encoding a member of the elongation factor complex, preferably a translation elongation factor, preferably eukaryotic translation elongation factor 1 alpha 1 (EEF1A1).

Other aspects and embodiments of the invention are described in more detail below.

### BRIEF DESCRIPTION OF FIGURES

FIG. 1—EEF1a Chromosomal location. Human EEF1a is located on chromosome 6 and is composed of 8 exons.

FIG. 2—ADP A2M4 knock-in strategy and PCR screening strategy—The targeting strategy was deigned to knock ADP A2M4 SPEAR into EEF1a1. Editing was performed with AAV. The homology arms in the rAAV repair template correspond the 500 bp of genomic sequence upstream and downstream of the stop codon. Editing creates a multicistronic fusion between EEF1a and ADP A2M4 with each polypeptide coding sequence separated by a furin cleavage site (RAKR) and 2A skip peptide (EEF1A1_FuT2A_TCRα_FuP2A_TCRβ). This design permits the expression of all 3 polypeptides from a single edited allele. The resistance cassette contains a LOXP flanked puromycin resistance gene constitutively expressed from the EF1A promoter PCR screening strategy. Edited clones were isolated by limiting dilution in 96 well plates. A sample of gDNA was isolated from each clone using QuickExtract™. Clones were screened by PCR with primers outside the homology arm regions and within the transgene cassette. The LHA boundary PCR was performed using primers 1824 and 1825. The RHA boundary PCR was performed with primers 2294 and 2295 PCR products were sequenced. Four clones were progressed for differentiation: 5F4, 5H4, 3D10, and 4B11

FIG. 3—Genotype of RAG1 knockout in the iPSC clone T1E8 (B2M HSV-TK/HLA-E EEF1A1 +/A2M4 RAG1 −/−). One allele contains a single nucleotide deletion whereas the second contains a 13 nucleotide deletion. Both edits result in a frameshift in the coding sequence of RAG1. The region of sequence targeted for editing is highlighted.

FIG. 4—Phenotypes of iT cells differentiated from edited iPSC clones (Stage 5): Differentiation is unaffected by editing. Edited clones express the ADP A2M4 SPEAR. Cells were phenotyped by FACS and A2M4 expression measured by staining with anti-Vα24 antibodies and ADP A2M4 MAGE-4 dextramer. Knock-in at a single allele is sufficient to promote ADP A2M4 expression.

FIG. 5—iT cells differentiated from edited iPSC clones exhibit specific and potent cytolytic effector function: iT cell cytolytic function was assessed with the KILR assay. T2 cells were pulsed with decreasing concentrations (10−5 M−10−11 M) of the MAGE-A4 derived peptide GVYDGREHTV and incubated with iT cells differentiated from the edited clones.

FIG. 6—Phenotypes of EEF1α+/A2M4 iT cells (clone 3E10) differentiated from edited iPSC clones (Stage 6): Differentiation is unaffected by editing. Edited clones express the ADP A2M4 SPEAR. Cells were phenotyped by FACS and show the ability of edited clones to differentiate into a CD8 T-Cell. Knock-in at a single allele is sufficient to promote ADP A2M4 expression.

FIG. 7—Phenotypes of RAG1−/−EEF1α+/A2M4 iT cells (clone T1E8) differentiated from edited iPSC clones (Stage 6): Differentiation is unaffected by the removal of RAG1 and cells were able to express the ADP A2M4 SPEAR. Cells were phenotyped by FACS and show the ability of edited clones to differentiate into a CD8 T-Cell. Knock-in at a single allele is sufficient to promote ADP A2M4 expression.

FIG. 8—target cell killing kinetics by EEF1α+/A2M4 iT cells in a time-resolved cytotoxicity assay. Left panel shows that EEF1α+/A2M4 iT cells (c3E10) have increased ability to induce apoptosis in A375 melanoma (antigen positive) target cells compared to controls in the presence or absence of MAGE-A4 derived peptide GVYDGREHTV. Right panel shows that EEF1α+/A2M4 iT cells (c3E10) have increased ability to induce apoptosis in COLO205 (antigen negative) target cells compared to controls in the presence but not in the absence of MAGE-A4 derived peptide GVYDGREHTV.

FIG. 9—target cell killing kinetics by RAG^{-/-}EEF1α^{+/A2M4} iT cells in a time-resolved cytotoxicity assay. Left panel shows that RAG^{-/-}EEF1α^{+/A2M4} iT cells (T1E8) have an increased ability to induce apoptosis in A375 melanoma (antigen positive) target cells compared to controls in the presence or absence of MAGE-A4 derived peptide GVYDGREHTV. Right panel shows that RAG^{-/-}EEF1α^{+/A2M4} iT cells (T1E8) have increased ability to induce apoptosis in COLO205 (antigen negative) target cells compared to controls in the presence but not in the absence of MAGE-A4 derived peptide GVYDGREHTV.

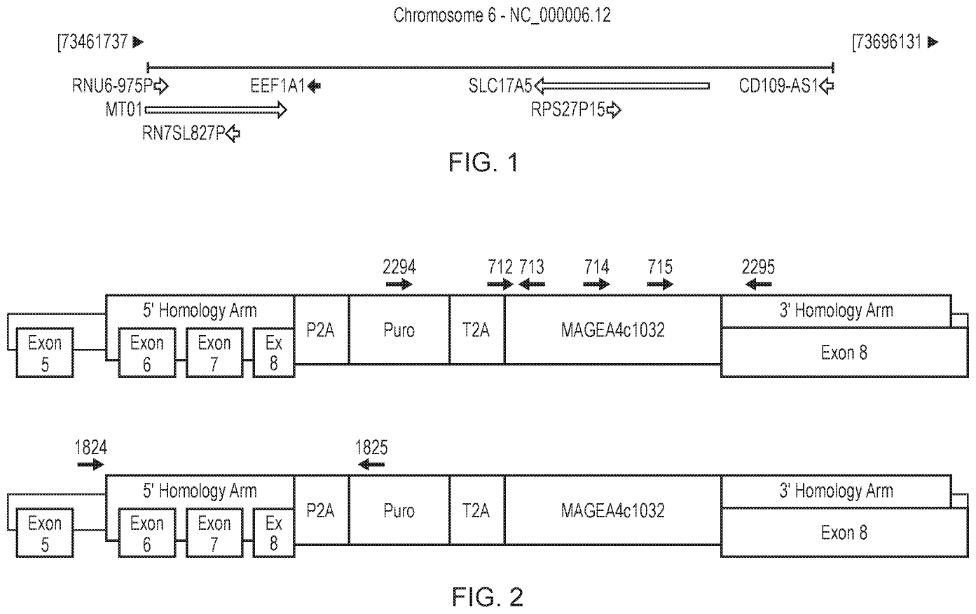

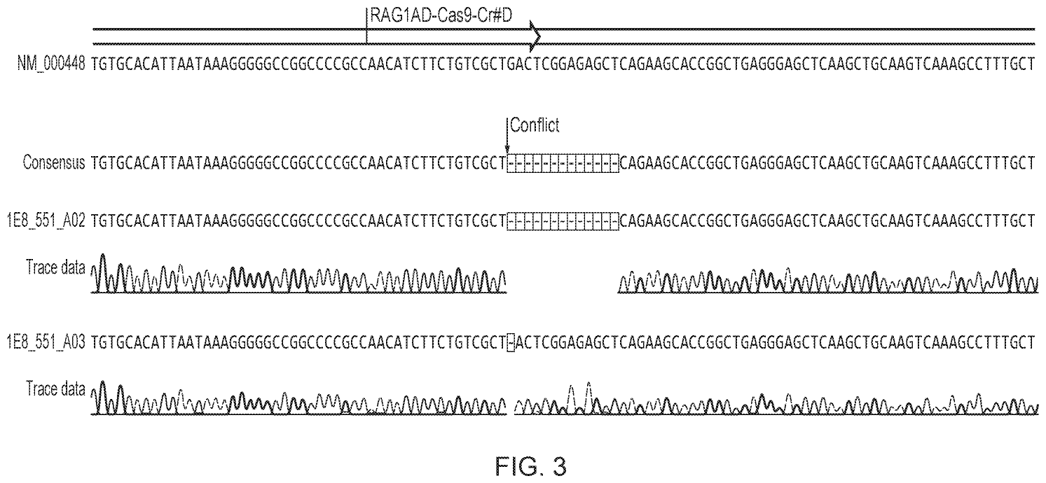

### DETAILED DESCRIPTION

Aspects and embodiments of the present invention will now be discussed with reference to the accompanying figures.

According to the present invention, there is provided a modified induced pluripotent stem cell (iPSC) or haemogenic lineage cell comprising at least one heterologous nucleic acid sequence encoding a heterologous T-cell receptor (TCR) integrated at or into a locus in the cell genome.

Integration of the nucleic acid sequence encoding the heterologous TCR at the locus allows the consistent generation of cells that express the heterologous TCR and reduces the need for screening compared to cells generated by random insertion.

The locus in the cell genome is or is in a gene encoding a member of the elongation factor complex. Preferably, the elongation factor complex is a translation elongation factor, most preferably eukaryotic translation elongation factor 1 alpha 1 (EEF1A1). In some especially preferred embodiments, the locus is at exon 8 of the eukaryotic translation elongation factor 1 alpha 1 (EEF1A1) gene, optionally before the TAG stop codon or immediately adjacent to and/or before the TAG stop codon. Exon 8 is the last exon of the human EEF1A1 gene and includes 40 full codons before the TGA stop codon. In some embodiments, the integration (insertion) locus may be between the last non-stop codon and the stop codon of the EEF1A1 gene. In other embodiments, the integration locus may be within one of the non-stop codons within exon 8, or between two adjacent non-stop codons within exon 8. Such an integration may lead to deletion of the downstream endogenous codons from the transcribed mRNA. However, if the deleted sequence is short, for example 1 to 10 non-stop codons, then such a short deletion of the C-terminal fragment may not affect the activity of the EEF1A1 protein. In some embodiments, the deleted non-stop codons (whether full or partial) may be included in the N-terminus of the inserted heterologous nucleic acid, such that the non-stop codons are included, in frame, in the transcript.

In other embodiments, the integration locus may be in one of the upstream exons of the EEF1A1 gene, such as exon 7, exon 6, exon 5, exon 4, exon 3, exon 2, or exon 1, so long as the truncated EEF1A1 transcript, along with the inserted heterologous nucleic acid which is designed to include the deleted portion, collectively expresses a functional EEF1A1 protein.

Integration at the EEF1A1 gene locus is shown herein to provide consistent high levels of expression of the heterologous TCR encoded by the heterologous nucleic acid sequence.

Other examples of members of the elongation factor complex include, for instance, EEF1A2 (Elongation factor 1-alpha 2; Entrez Gene ID for the human gene: 1917), EEF1A1P43 (Eukaryotic translation elongation factor 1 alpha 1 pseudogene 43; Entrez Gene ID for the human gene: 1918), EEF1B2 (Elongation factor 1-beta; Entrez Gene ID for the human gene: 1933), EEF1B2P1 (Eukaryotic translation elongation factor 1 beta 2 pseudogene 1; Entrez Gene ID for the human gene: 1932), eEF1B1 (Eukaryotic translation elongation factor 1 beta 2 pseudogene 1; Entrez Gene ID for the human gene: 1932), EEF1B2P2 (Eukaryotic translation elongation factor 1 beta 2 pseudogene 2; Entrez Gene ID for the human gene: 1934), EEF1B2P3 (Eukaryotic translation elongation factor 1 beta 2 pseudogene 3; Entrez Gene ID for the human gene: 644820), EEF1G (Elongation factor 1-gamma; Entrez Gene ID for the human gene: 1937), EEF1D (Elongation factor 1-delta; Entrez Gene ID for the human gene: 1936), EEF1E1 (Eukaryotic translation elongation factor 1 epsilon-1; Entrez Gene ID for the human gene: 9521), VARS (Valyl-tRNA synthetase; Entrez Gene ID for the human gene: 7407), EEF2 (Eukaryotic elongation factor 2; Entrez Gene ID for the human gene: 1938), and EIF5A (Eukaryotic translation initiation factor 5A-1; Entrez Gene ID for the human gene: 1984).

In some embodiments, the integration locus is in the last exon, such as between the last non-stop codon and the stop codon; alternatively, as explained above, the integration locus may be more upstream within the last exon, or in an upstream exon, preferably without interrupting the expression and activity of the gene at the locus.

Integration of the nucleic acid sequence encoding the heterologous TCR at the locus may generate a multicistronic fusion that comprises nucleic acid sequence encoding the member of the elongation factor complex and nucleic acid sequence encoding the heterologous TCR. The multicistronic fusion may comprise one or more further nucleic acid sequences that allows expression of the member of the elongation factor complex and the heterologous TCR from the multicistronic fusion. For example, the multicistronic fusion may comprise one or more nucleic acid sequences encoding an enzymatic cleavage site and/or nucleic acid sequences which mediate ribosome-skipping.

The at least one heterologous nucleic acid sequence encoding a heterologous TCR may be an expressible heterologous nucleic acid sequence. For example, the heterologous nucleic acid sequence may comprise one or more encoding genes or one or more encoding open reading frames and/or one or more regulatory nucleic acid sequences capable of enabling and/or regulating the expression of the one or more encoding genes or one or more encoding open reading frames within the cell. Suitable regulatory nucleic acid sequences include sequences enabling the transcription and/or post transcriptional modification and/or translation of the encoding gene or genes or open reading frames. Suitable regulatory sequences may comprise any of transcriptional or translational start, stop or termination sequences, operator sequence, promoter sequence, enhancer sequence, terminal repeats, ribosome binding site, cap sequence and polyA tail sequence.

The technology described herein, is not limited to the production of cells that express a heterologous/exogenous TCR and may be adapted to produce cells that express other therapeutic proteins, such as chimeric antigen receptors (CAR). A T cell that expresses a CAR is commonly referred to as a CAR-T cell. A natural killer (NK) cell that expresses a CAR is commonly referred to as a CAR-NK cell. A macrophage that expresses a CAR is commonly referred to as a CAR-macrophage. As described, a heterologous nucleic acid that encodes a fusion protein that includes one or more protease cleavage site(s) and a CAR molecule may be integrated to a target cell, such as an iPSC, which can then be differentiated into a target immune cell. Alternatively, the integration may be carried out with the immune cell directly.

The term “modified iPSC or haemogenic lineage cell” may be used herein to describe cells at any stage in differentiation that have been modified to incorporate the heterogenous nucleic acid in the EEF1A1 locus. For example, a modified iPSC or haemogenic lineage cell may be a cell selected from the group consisting of an iPSC, a mesoderm cell, a haemogenic endothelial cell, a haematopoietic progenitor cell, a progenitor T cell, a double positive T cell (DP T cell), a single positive T cell (SP T cell) and a mature T cell. Preferably, the modified iPSC or haemogenic lineage cell according to the invention, such as a T cell, expresses or presents the at least one heterologous TCR encoded by a heterologous TCR coding sequence, preferably expressed or presented at the cell surface. Preferably the at least one heterologous TCR is presented at the cell surface as a membrane bound functional TCR, preferably a membrane anchored heterodimeric TCR protein, preferably an alpha: beta TCR (optionally gamma: delta), preferably comprising variable alpha (α) and beta (β) chains, preferably expressed as a complex with invariant CD3 chains molecules. Preferably, the at least one heterologous TCR is capable of binding or specifically binding to a cancer and/or tumour antigen or peptide thereof and/or to tumour and/or cancer cells (e.g. expressing a cancer and/or tumour antigen or peptide thereof) and/or to peptides or antigenic peptides therefrom. Preferably, the binding of the TCR promotes activation of the modified iPSC or haemogenic lineage cell as herein described, preferably a T cell, and/or activation of T cell activity as herein described.

According to the present invention, the modified haemogenic lineage cell, preferably a T cell, may be derived from the modified iPSC. For example, an unmodified iPSC may be modified or engineered, for example recombinantly engineered, to comprise the at least one heterologous nucleic acid sequence encoding a heterologous T-cell receptor (TCR) integrated at or into a locus in the cell genome, for example to produce the modified induced pluripotent stem cell which may then be differentiated into a modified haemogenic lineage cell. Alternatively, an unmodified haemogenic lineage cell may be derived from an unmodified iPSC, for example by a process of differentiation, and then may be modified or engineered to comprise the at least one heterologous nucleic acid sequence encoding a heterologous T-cell receptor (TCR) integrated at or into a locus in the cell genome.

The “iPSC or haemogenic lineage cell”, or the “modified iPSC or haemogenic lineage cell” may be selected from any one of:
(a) a mesoderm cell, optionally which may express any one or more of the mesodermal markers, Brachyury, Goosecoid, MixI1, KDR (also known as FLK1 or VEGFR2), FoxA2, GATA6 or PDGF alpha R,
(b) a haemogenic endothelial cell, optionally which may be (a) CD34+ or (b) CD34+ CD73− or (c) CD34+ CD73− CXCR4− (CD184−),
(c) a haematopoietic progenitor cell, optionally which may be (a) CD34+ or (b) CD34+ CD45+ or (c) CD34+ and/or CD45+ in combination with any one or more of CD117+, CD133+, CD45+, FLK+, CD38−, (d) CD34+, CD133+, CD45+, FLK1+, CD38−,
(d) a progenitor T cell, optionally which may be (a) CD5+ and/or CD7+, or (b) CD5+ and/or CD7+ in combination with any one or more of: CD44+, CD25+, CD2+, CD45+, CD3−, CD4−, CD8−,
(e) a double positive T cell (DP T cell), optionally which may be (a) CD4+ CD8+ (b) CD4+ CD8+ in combination with any one or more of CD3+, CD28+, CD45+,
(f) a single positive T cell (SP T cell) optionally which may be (a) CD4+ (b) CD8+ (b) CD4+ or CD8+ in combination with any one or more of CD3+, CD28+, CD45+,
(g) a mature T cell, optionally alpha beta T cell, gamma delta T cell, NKT cell, T helper cell (TH) which are CD4+, cytotoxic T cell (TC) which are CD8+, or
(h) an iPSC, optionally a human iPSC derived or created from a CD34+ progenitor cell, optionally isolated from umbilical cord blood, further optionally using the pEB-C5 and pEB-TG episomal plasmids.

Accordingly, the iPSC is a human iPSC, preferably derived or created from a CD34+ progenitor cell, optionally preferably isolated from umbilical cord blood, further optionally isolated using pEB-C5 and/or pEB-TG episomal plasmids.

In some embodiments of the invention, the iPSC may be modified to reduce or eliminate RAG1 and/or RAG2 expression, for example by inactivation or knock-out of the RAG1 and/or RAG2 gene. RAG1 and RAG2 knockout can be achieved by any suitable gene editing technique, including CRISPR-Cas9, to introduce suitable inactivating mutations such as frameshifts or stop codons.

The modified pluripotent stem cell iPSC or haemogenic lineage cells may be cells of the lymphoid lineage including T cells, Natural Killer T (NKT) cells, and precursors thereof including embryonic stem cells, and pluripotent stem cells (e.g. those from which lymphoid cells may be differentiated). T cells can be lymphocytes that mature in the thymus and are chiefly responsible for cell-mediated immunity and also involved in the adaptive immune system. According to the present invention, the T cells can include, but are not limited to, helper T cells, cytotoxic T cells, memory T cells (including central memory T cells, stem-cell-like memory T cells (or stem-like memory T cells)), and two types of effector memory T cells: e.g., TEM cells and TEMRA cells, Regulatory T cells (also known as suppressor T cells), Natural killer T cells, Mucosal associated invariant T cells, and gamma-delta T cells.

Cytotoxic T cells (CTL or killer T cells) are a subset of T-lymphocytes capable of inducing the death of infected somatic or tumour cells. Preferably, the T cells are optionally a CD4+ T cell or a CD8+ T cell or population of such T-cells, optionally CD4+ T cells; or CD8+ T cells, or a mixed population of CD4+ T cells and CD8+ T cells.

The heterologous nucleic acid sequence encoding the heterologous TCR may be integrated at or into a locus in the cell genome, i.e. the genome of the iPSC or haemogenic lineage cell. The locus may be a specific and/or fixed position on a chromosome for example where a particular gene or genetic marker is located, it may be the specific and/or fixed position of a gene or genetic marker or genetic element and/or a specific fixed position within a gene or genetic element of a gene such as, for example, an intron or exon of the gene or a regulatory element of a gene (e.g. transcriptional or translational start, stop or termination sequences, operator sequence, promoter sequence, enhancer sequence, terminal repeats, ribosome binding site, cap sequence, polyA tail sequence).

The heterologous nucleic acid sequence encoding a heterologous TCR may be integrated at or into one or both alleles of the locus in the cell genome. For example, where the locus is a gene or endogenous gene or a specific and/or fixed position on a chromosome, and/or the position where a particular gene or endogenous gene of the iPSC or haemogenic lineage cell is located, then the heterologous nucleic acid sequence encoding a heterologous TCR may be integrated at either one or both alleles of the locus.

Accordingly, the locus may be a gene or may be within a gene encoding an endogenous protein of the cell or the iPSC or haemogenic lineage cell.

The heterologous nucleic acid sequence encoding a heterologous TCR may be integrated adjacent to or within a gene encoding an endogenous protein of the cell or the iPSC or haemogenic lineage cell. Optionally the heterologous nucleic acid sequence encoding a heterologous TCR may be integrated within an intron or exon of a gene or nuclei acid sequence encoding an endogenous protein of the cell or the iPSC or haemogenic lineage cell, optionally a 5′ exon or 3′ exon, preferably 3′ exon.

The heterologous nucleic acid sequence encoding a heterologous TCR may be integrated within the 3′ exon before the TAG stop sequence of the gene encoding the endogenous protein. Preferably, the integration of the heterologous nucleic acid sequence encoding a heterologous TCR, is non-disruptive to the production of the endogenous protein. For example, the production of the endogenous protein by the modified cell should provide all or substantially all of the endogenous protein sequence, which may be translated and/or produced and/or processed equivalently to the native protein. For example, the gene encoding the endogenous protein may still be capable of transcription and/or post transcriptional processing and/or translation to produce the endogenous protein sequence or substantially all of the endogenous protein sequence which may optionally be post translationally processed and/or trafficked in the cell. Preferably, the integration of the heterologous nucleic acid sequence encoding a heterologous TCR, does not result in the deletion or substitution of the gene encoding the endogenous protein, and/or the deletion and/or inactivation of the produced endogenous protein, it may result in the concatenation of the heterologous nucleic acid sequence encoding a heterologous TCR with the nucleic acid sequence or gene encoding the endogenous protein, optionally such that a fusion gene results and/or the gene products are produced as a fusion protein.

The modified iPSC or haemogenic lineage cell may comprise a fusion sequence between the nucleic acid encoding the heterologous TCR and the nucleic acid encoding the endogenous protein, for example as generated by the integration of the nucleic acid encoding the heterologous TCR, for example a fusion gene or sequence or multicistronic fusion gene or sequence between the nucleic acid encoding the heterologous TCR and the nucleic acid encoding the endogenous protein, for example wherein the expression of the heterologous TCR is linked to the expression of the endogenous protein and/or wherein the heterologous TCR may co-transcribed and/or co-translated and/or co-expressed with the gene product of the gene at the locus, e.g. the nucleic acid encoding the endogenous gene.

Accordingly, the invention provides a modified iPSC or haemogenic lineage cell in which the nucleic acid encoding the heterologous TCR may be connected to a nucleic acid encoding an endogenous protein by a nucleic acid sequence encoding a peptide comprising an enzymatic cleavage site and/or a nucleic acid sequence which mediates ribosome-skipping.

Accordingly, the invention provides a modified iPSC or haemogenic lineage cell in which the nucleic acid encoding the heterologous TCR may be connected to a nucleic acid encoding an endogenous protein by at least one, optionally two, nucleic acid sequence encoding a peptide comprising an enzymatic cleavage site and/or at least one, optionally two, nucleic acid sequence which mediates ribosome-skipping. The nucleic acid sequence which mediates ribosome-skipping may be either a T2A and/or P2A skip sequence. The nucleic acid sequence encoding a peptide comprising an enzymatic cleavage site may encode a furin cleavage site, the sequence of the encoded furin site may be preferably RAKR.

The nucleic acid encoding the heterologous TCR may comprise a coding sequence of a TCRα and/or TCRβ chain, optionally with at least one, optionally two, intervening nucleic acid sequences encoding a peptide comprising an enzymatic cleavage site and/or at least one, optionally two, nucleic acid sequence which mediates ribosome-skipping. The nucleic acid sequence which mediates ribosome-skipping may be either a T2A and/or P2A skip sequence. The nucleic acid sequence encoding a peptide comprising an enzymatic cleavage site may encode a furin cleavage site. The sequence of the encoded furin site may be preferably RAKR.

Accordingly, the transcription and/or expression of the heterologous TCR and the endogenous protein of the modified iPSC or haemogenic lineage cell according to the invention may be from the same promoter. Accordingly, the heterologous nucleic acid sequence encoding a heterologous TCR and nucleic acid sequence or gene encoding the endogenous protein may share the same regulatory sequences for transcription and/or post transcriptional modification or co-transcriptional modification and/or translation, regulatory sequences as referred to herein. Post transcriptional modification or co-transcriptional may include processing of an RNA primary transcript following transcription to produce a mature, functional RNA molecule and/or may include any of mRNA processing and/or 5′ processing and/or capping and/or 3′ processing and/or cleavage and polyadenylation and/or introns splicing and/or histone mRNA processing.

A modified iPSC or haemogenic lineage cell according to the invention may comprise a heterologous TCR that is expressed as a fusion protein with an endogenous protein, optionally connected by a peptide comprising an enzymatic cleavage site, preferably a furin cleavage site, preferably RAKR and/or ribosome skip sequence, preferably T2A and/or P2A skip sequence.

Accordingly, the fusion protein may be processed in the cell to release the nascent, free or native heterologous TCR, optionally by cleavage of the enzyme cleavage site or furin cleavage site and/or removal the skip sequence peptides optionally performed in the endoplasmic reticulum of the cell.

The present invention provides a modified iPSC or haemogenic lineage cell according to the invention, such as a T cell, wherein the heterologous TCR is expressed and/or presented in the cell as a nascent heterologous TCR comprising a TCR alpha chain and TCR beta chain, preferably expressed and/or presented at the cell surface. The nascent protein may be separated from the fusion protein and thereby from the endogenous protein for example during post translational processing in the cell, such as in the cell endoplasmic reticulum or Golgi, it is then optionally trafficked and/or presented at the cell surface as a membrane bound functional TCR, preferably a membrane anchored heterodimeric TCR protein, preferably an alpha; beta TCR optionally capable of binding or specifically binding to a cancer or tumour antigen or peptide thereof and/or to tumour and/or cancer cells and/or tumour and/or cancer tissue and/or to peptides or antigenic peptides therefrom and/or promoting activation of the modified iPSC or haemogenic lineage cell, such as a T cell, as herein described and/or activating T cell activity as herein described.

According to the present invention, the endogenous protein can be a member of the elongation factor complex, preferably a translation elongation factor, preferably eukaryotic translation elongation factor 1 alpha 1 optionally wherein the locus is at exon 8 of the eukaryotic translation elongation factor 1 alpha 1 (EEF1A1) gene, optionally before the TAG stop codon or immediately adjacent to and/or before the TAG stop codon.

The at least one heterologous nucleic acid sequence encoding a heterologous T-cell receptor (TCR) can be integrated at or into a locus in the cell genome where the locus is or is in a gene encoding a member of the elongation factor complex, preferably a translation elongation factor, preferably eukaryotic translation elongation factor 1 alpha 1 optionally wherein the locus is at exon 8 of the eukaryotic translation elongation factor 1 alpha 1 (EEF1A1) gene, optionally before the TAG stop codon or immediately adjacent to and/or before the TAG stop codon.

The at least one heterologous nucleic acid sequence encoding a heterologous T-cell receptor (TCR) can be integrated at or into a locus in the cell genome where the locus or site of integration is at exon 8 of the EEFA1A gene, preferably in the EEFA1A gene on chromosome 6, optionally before the TAG stop codon or immediately adjacent to and/or before the TAG stop codon.

The present invention provides a modified iPSC or haemogenic lineage cell according to the present invention, wherein the fusion gene or sequence or multicistronic fusion gene or sequence comprises the nucleic acid encoding the heterologous TCR and nucleic acid encoding EEFA1A with an intervening nucleic acid sequence encoding a peptide comprising an enzymatic cleavage site, preferably a furin cleavage site, and/or nucleic acid sequence which mediates ribosome-skipping, preferably selected from a T2A and/or P2A skip sequence and wherein the nucleic acid sequence encoding the heterologous TCR comprises the coding sequence of a TCRα and TCRβ chain, preferably with an intervening nucleic acid sequence encoding a peptide comprising an enzymatic cleavage site, preferably a furin cleavage site, and/or nucleic acid sequence which mediates ribosome-skipping, preferably selected from T2A and/or P2A skip sequence.

TCR Binding

The heterologous TCR may bind or specifically bind to an antigen or peptide antigen which is any of:
(a) a cancer and/or tumour antigen or peptide antigen thereof, or
(b) a cancer and/or tumour antigen or peptide antigen thereof associated with a cancerous condition and/or presented by tumour or cancer cell or tissue.

According to the present invention, the heterologous TCR may bind or specifically and/or selectively bind to an antigen or peptide antigen thereof, for example to a cancer and/or tumour antigen or peptide antigen thereof, optionally presented by a cancer and/or tumour and/or in complex with a peptide presenting molecule, for example major histocompatibility complex (MHC) or an HLA, alternatively presented without a peptide presenting molecule (for example as an endogenously expressed cancer and/or tumour cell surface antigen or peptide antigen). The cancer and/or tumour antigen or peptide antigen thereof can be an antigen or peptide antigen thereof expressed by cancer and/or tumour cells or tissue at a higher level than on normal cells or tissue, preferably a significantly higher level, for example 10, 100, 1000, 10,000, 100,000, 1000,000 or greater times higher, preferably the antigen or peptide antigen thereof is not expressed by normal cells or tissue. The cancer and/or tumour antigen or peptide antigen thereof may be any of a cancer-testis antigen, NY-ESO-1, MART-1 (melanoma antigen recognized by T cells), WT1 (Wilms tumor 1), gp 100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (α-fetoprotein), a MAGE, melanoma associated antigen or member of the MAGEA gene family, MAGE-A1, MAGE-A2, MAGE-A3, MAGE-A4, MAGE-A6, MAGE-A8, and MAGE-A9, MAGE-A10, or MAGE-A12, or peptide antigen thereof.

The TCR may bind MAGE A4, or an antigenic peptide thereof, for example Human MAGE A4 or MAGE A4 of SEQ ID NO: 1 or an antigenic peptide thereof. The TCR may bind to an antigenic peptide of MAGE A4 comprising SEQ ID NO: 2, GVYDGREHTV. Alternatively, the TCR may bind alpha fetoprotein (AFP), or an antigenic peptide thereof, for example alpha fetoprotein (AFP) of SEQ ID NO: 20 or a peptide antigen of AFP or a peptide antigen of AFP comprising FMNKFIYEI (SEQ ID No: 21) or residues 158-166 derived from alpha fetoprotein (AFP) SEQ ID NO: 20.

The TCR may bind to an AFP antigenic peptide comprising or consisting of;
(a) residues 158-166 of SEQ ID NO:20 (AFP158),
(b) residues 137-145 of SEQ ID NO:20 (AFP137),
(c) residues 325-334 of SEQ ID NO:20 (AFP325)
(d) residues 542-550 of SEQ ID NO:20 (AFP542), or
(e) residues FMNKFIYEI (SEQ ID No: 21).

### Specific Binding TCR

The heterologous TCR may bind or specifically bind an antigen or peptide antigen thereof for example a cancer and/or tumour antigen or peptide antigen thereof as described herein, optionally associated with a cancerous condition and/or presented by tumour or cancer cell or tissue.

Specificity describes the strength of binding between the heterologous TCR and a specific target cancer and/or tumour antigen or peptide antigen thereof and may be described by a dissociation constant, Kd, the ratio between bound and unbound states for the receptor-ligand system. Additionally, the fewer different cancer and/or tumour antigens or peptide antigen thereof the heterologous TCR can bind, the greater its binding specificity.

The heterologous TCR may bind to less than 10, 9, 8, 7, 6, 5, 4, 3 or 2 different cancer and/or tumour antigens or peptide antigen thereof.

The heterologous TCR may bind, an antigen or peptide antigen thereof, for example to a cancer and/or tumour antigen or peptide antigen, for example MAGE A4 of SEQ ID NO: 1, an antigenic peptide of MAGE A4 comprising SEQ ID NO: 2, alpha fetoprotein (AFP) of SEQ ID NO: 20 or a peptide antigen of AFP or a peptide antigen of AFP comprising FMNKFIYEI (SEQ ID No: 21), with a dissociation constant of between: 0.01 μM and 100 μM, between 0.01 μM and 50 μM, between 0.01 μM and 20 μM, between 0.05 μM and 20 μM or of 0.01, 0.02, 0.03, 0.04, 0.05, 0.06, 0.07, 0.08, 0.09, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5μM, 7.0 μM, 7.5μM, 8.0 μM, 8.5 μM, 9.0 μM, 9.5 μM, 10.0 μM; or between 10 μM and 1000 μM, between 10 μM and 500 μM, between 50 μM and 500 μM or of 10, 20 30, 40, 50 60,70, 80, 90, 100 μM, 150 μM, 200 μM, 250 μM, 300 μM, 350 μM, 400 μM, 450 μM, 500 μM; optionally measured with surface plasmon resonance, optionally at 25° C., optionally between a pH of 6.5 and 6.9 or 7.0 and 7.5. The dissociation constant, KD or koff/kon may be determined by experimentally measuring the dissociation rate constant, koff, and the association rate constant, kon. A TCR dissociation constant may be measured using a soluble form of the TCR, wherein the TCR comprises a TCR alpha chain variable domain and a TCR beta chain variable domain.

The heterologous T cell receptor (TCR), and/or modified cells comprising the heterologous T cell receptor (TCR) may specifically and/or with high affinity, and/or selectively bind, an antigen or peptide antigen thereof, for example to a cancer and/or tumour antigen or peptide antigen, as herein described in complex with or presented by a peptide presenting molecule for example major histocompatibility complex (MHC) or an HLA, optionally class I or II, for example with HLA-A2, or selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642; for example with a dissociation constant as herein above described, preferably of between 0.01 μM and 100 μM such as 50 μM, 100 μM, 200 μM, 500 μM, preferably between 0.05 μM to 20.0 μM.

The heterologous T cell receptor (TCR), and/or modified cells comprising the heterologous T cell receptor (TCR) may specifically and/or with high affinity, and/or selectively bind, MAGE A4 (SEQ ID NO: 1) a peptide antigen thereof or an antigenic peptide thereof comprising SEQ ID NO: 2, GVYDGREHTV optionally in complex with HLA-A*02, optionally selected from HLA*02, HLA-A*02:01, HLA-A*02:02, HLA-A*02:03, HLA-A*02:04, HLA-A*02:06, HLA-A*02:642 or HLA-A*02:07, preferably HLA-A*02:01 or HLA-A*02; for example with a dissociation constant as herein above described, preferably of between 0.01 μM and 100 μM such as 50 μM, 100 μM, 200 μM, 500 μM, preferably between 0.05 μM to 20.0 μM.

The heterologous T cell receptor (TCR), and/or modified cells comprising the heterologous T cell receptor (TCR) may specifically and/or with high affinity, and/or selectively bind, to AFP (SEQ ID NO: 20) a peptide antigen thereof or peptide antigen comprising the sequence FMNKFIYEI (SEQ ID No: 21), optionally in complex with HLA-A2, or with any HLA selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642; for example with a dissociation constant as herein above described, preferably of between 0.01 μM and 100 μM such as 50 μM, 100 μM, 200 μM, 500 μM, preferably between 0.05 μM to 20.0 μM.

Alternatively, the heterologous T cell receptor (TCR), and modified cells comprising the heterologous T cell receptor (TCR) may bind or specifically and/or selectively bind and/or bind with high affinity to an antigen or peptide antigen thereof, for example to a cancer and/or tumour antigen or peptide antigen, as herein described as an endogenously expressed cancer and/or tumour cell surface antigen or peptide antigen (e.g. AFP or MAGE A4 or antigenic peptides thereof) optionally wherein the binding is independent of presentation of the cell surface antigen as a complex with an peptide-presenting or antigen-presenting molecule, for example major histocompatibility complex (MHC) or human leukocyte antigen (HLA) or major histocompatibility complex class related protein (MR) 1; for example with a dissociation constant as herein above described, preferably of between 0.01 μM and 100 μM such as 50 μM, 100 μM, 200 μM, 500 μM, preferably between 0.05 μM to 20.0 μM.

TCR binding may be specific for one antigen or peptide antigen thereof, for example a cancer and/or tumour antigen or peptide antigen thereof, as herein described (e.g. AFP or MAGE A4 or antigenic peptides thereof), optionally as expressed on a cancer and/or tumour cell surface, in comparison to a closely related cancer and/or tumour antigen or peptide antigen sequence. The closely related cancer and/or tumour antigen or peptide antigen sequence may be of similar or identical length and/or may have a similar number or identical number of amino acid residues. The closely related peptide antigen sequence may share between 50 or 60 or 70 or 80 to 90% identity, preferably between 80 to 90% identity and/or may differ by 1, 2, 3 or 4 amino acid residues. For example, the closely related peptide sequence may be derived from the polypeptide sequence comprising the sequence or having the sequence GVYDGREHTV, SEQ ID NO: 2 or FMNKFIYEI, SEQ ID No: 21.

The binding affinity may be determined by equilibrium methods (e.g. enzyme-linked immunosorbent assay (ELISA) or radioimmunoassay (RIA)), or kinetics (e.g. BIACORE™ analysis). Avidity is the sum total of the strength of binding of two molecules to one another at multiple sites, e.g. taking into account the valency of the interaction. The modified pluripotent stem cell iPSC or haemogenic lineage cells may demonstrate improved affinity and/or avidity to a cancer and/or tumour antigen or peptide antigen thereof, or a cancer and/or tumour antigen or peptide antigen thereof presented by tumour of cancer cell or tissue and recognized by the heterologous TCR in comparison to cells lacking the heterologous TCR or having an alternative heterologous TCR or CAR.

Selective binding denotes that the heterologous TCR binds with greater affinity to one cancer and/or tumour antigen or peptide antigen thereof in comparison to another. Selective binding is denoted by the equilibrium constant for the displacement by one ligand antigen of another ligand antigen in a complex with the heterologous TCR.

### Heterologous TCR

A modified iPSC or haemogenic lineage cell, such as a T cell, comprising at least one heterologous nucleic acid sequence encoding a heterologous TCR integrated into a locus in the cell genome may express a heterologous T cell receptor (TCR). Upon binding to the antigen, a modified iPSC or haemogenic lineage cell, such as a T cell, can exhibit T cell effector functions and/or cytolytic effects towards cells bearing the antigen and/or undergo proliferation and/or cell division. In certain embodiments, the modified iPSC or haemogenic lineage cell, such as a T cell, comprising the TCR exhibits comparable or better therapeutic potency compared to T cells comprising either a transduced TCR or a chimeric antigen receptor (CAR) targeting the same cancer and/or tumour antigen and/or peptide (antigenic peptide). Activated modified iPSCs or haemogenic lineage cells, such as T cells, can secrete anti-tumour cytokines which can include, but are not limited to, TNFalpha, IFNγ and IL2.

The term “heterologous” or “exogenous” refers to a polypeptide or nucleic acid that is foreign to a particular biological system, such as a cell or host cell, and is not naturally present in that system and which may be introduced to the system by artificial or recombinant means. Accordingly, the expression of a TCR which is heterologous, may thereby alter the immunogenic specificity of an iPSC or haemogenic lineage cell, for example a T cell, so that it recognises or displays improved recognition for one or more tumour or cancer antigens and/or peptides as herein described that are present on the surface of the cancer cells of an individual with cancer. The modification of immunogenic cells or T cells and their subsequent expansion may be performed in vitro and/or ex vivo.

### TCR Structure

Preferably, the heterologous TCR is not naturally or endogenously expressed by the iPSCs or haemogenic lineage cells, i.e. prior to modification (i.e., the TCR is exogenous or heterologous). A heterologous TCR may include a αβTCR heterodimer.

A heterologous TCR may be a recombinant or synthetic or artificial TCR i.e. a TCR that does not exist in nature. For example, a heterologous TCR may be engineered to increase its affinity or avidity for a specific cancer and/or tumour antigen or peptide antigen thereof (i.e. an affinity enhanced TCR or specific peptide enhanced affinity receptor (SPEAR) TCR). The affinity enhanced TCR or (SPEAR) TCR may comprise one or more mutations relative to a naturally occurring TCR, for example, one or more mutations in the hypervariable complementarity determining regions (CDRs) of the variable regions of the TCRα and β chains. These mutations may increase the affinity of the TCR for a cancer and/or tumour antigen or peptide antigen thereof, as herein described or MHCs that display a cancer and/or tumour antigen or peptide antigen thereof, as herein described, optionally when expressed by tumour and/or cancer cells. Suitable methods of generating affinity enhanced or matured TCRs include screening libraries of TCR mutants using phage or yeast display and are well known in the art (see for example Robbins et al J Immunol (2008) 180 (9): 6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to tumour or cancer cells expressing the tumour antigen of the MAGE family, or AFP or peptide antigens thereof as described herein.

### MAGEA4/AFP TCR

The heterologous TCR may be a MAGE A4 TCR, which may comprise the α chain reference amino acid sequence of SEQ ID NO: 5 or a variant thereof and the β chain reference amino acid sequence of SEQ NO: 7 or a variant thereof. Alternatively, the heterologous TCR may be an AFP TCR which may comprise the α chain reference amino acid sequence of SEQ ID NO: 22 or a variant thereof and the β chain reference amino acid sequence of SEQ NO: 23 or a variant thereof.

A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence (for example, with respect to either α chain reference sequence and/or β chain reference sequence). The TCR may be encoded by the α chain reference nucleotide sequence of SEQ ID NO: 6 or a variant thereof and the β chain reference nucleotide sequence of SEQ ID NO: 8 or a variant thereof (MAGE A4) or by the α chain reference nucleotide sequence of SEQ ID NO: 24 or a variant thereof and the β chain reference nucleotide sequence of SEQ ID NO: 25 (AFP) or a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence (for example, with respect to either α chain reference sequence and/or β chain reference sequence for an AFP or MAGE A4 TCR).

### TCR CDRs

According to the present invention, a heterologous TCR, for example for MAGE A4, may comprise a TCR alpha chain variable domain and a TCR beta chain variable domain, wherein:
the alpha chain variable domain comprises CDRs having the sequences VSPFSN (αCDR1), SEQ ID NO:11 or amino acids 48-53 of SEQ ID NO:5, LTFSEN (αCDR2), SEQ ID NO: 12 or amino acids 71-76 of SEQ ID NO:5, and CVVSGGTDSWGKLQF (αCDR3), SEQ ID NO: 13 or amino acids 111-125 of SEQ ID NO:5, and/or
the beta chain variable domain comprises CDRs having the sequences KGHDR (βCDR1), SEQ ID NO:14 or amino acids 46-50 of SEQ ID NO:7, SFDVKD (βCDR2), SEQ ID NO:15 or amino acids 68-73 of SEQ ID NO:7, and CATSGQGAYEEQFF (βCDR3), SEQ ID NO: 16 or amino acids 110-123 of SEQ ID NO:7 or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto, optionally 100% sequence identity thereto, respectively.

According to the invention, a heterologous TCR, for example for AFP, may comprise a TCR alpha chain variable domain and a TCR beta chain variable domain, wherein:
(i) the alpha chain variable domain comprises CDRs having the sequences DRGSQS (αCDR1), SEQ ID NO:26 or amino acids 27-32 of SEQ ID NO:22, or a sequence having at least 50% sequence identity thereto, IYSNGD (αCDR2), SEQ ID NO:27 or amino acids 50-55 of SEQ ID NO:22, or a sequence having at least 50% sequence identity thereto, and AVNSDSGYALNF (αCDR3), SEQ ID NO:28 or amino acids 90-101 of SEQ ID NO: 22, or a sequence having at least 50% sequence identity thereto, and/or
(ii) the beta chain variable domain comprises CDRs having the sequences SGDLS (βCDR1), SEQ ID NO:29 or amino acids 27-31 of SEQ ID NO:23, or sequence having at least 50% sequence identity thereto, YYNGEE (βCDR2), SEQ ID NO:30 or amino acids 49-54 of SEQ ID NO:23, or sequence having at least 50% sequence identity thereto, and ASSLGGESEQY (βCDR3), SEQ ID NO:31 or amino acids 92-102 of SEQ ID NO:23; or sequences having at least 50% sequence identity thereto; optionally the sequence identity may be any of least 50%, at least 55%, at least 60%, at least 65%, at least 70%, at least 75%, at least 80%, at least 85%, at least 90%, at least 95%, at least 98% or at least 99% sequence identity, optionally 100% sequence identity thereto respectively.

### TCR Variable Domains

The heterologous TCR may comprise a TCR, e.g. MAGE A4 TCR, in which the alpha chain variable domain comprises an amino acid sequence that has at least 50%, 55%, 60%, 65%, 70%, 75%, 80%, 85%, 90%, 95%, 96%, 97%, 98% or 99% or 100% identity to SEQ ID NO: 9 or the sequence of amino acid residues 1-136 of SEQ ID NO:5, and/or the beta chain variable domain comprising an amino acid sequence that has at least 50%, 55%, 60%, 65%, 70%, 75%, 80%, 85%, 90%, 95%, 96%, 97%, 98% or 99% or 100% identity to SEQ ID NO: 10 or the sequence of amino acid residues 1-133 of SEQ ID NO:7.

The heterologous TCR, e.g. AFP TCR, may comprise a TCR in which the alpha chain variable domain comprises an amino acid sequence that has at least 50%, 55%, 60%, 65%, 70%, 75%, 80%, 85%, 90%, 95%, 96%, 97%, 98% or 99% or 100% identity to the sequence of amino acid residues 1-112 of SEQ ID NO: 22, and/or the beta chain variable domain comprising an amino acid sequence that has at least 50%, 55%, 60%, 65%, 70%, 75%, 80%, 85%, 90%, 95%, 96%, 97%, 98% or 99% or 100% identity to the sequence of amino acid residues 1-112 of SEQ ID NO: 23.

### MAGE A4 & AFP TCR CDRs and Frameworks

In some embodiments, the terms “progenitor TCR” or “parental TCR”, may be used herein to refer to a TCR comprising the MAGE A4 TCRα chain and MAGE A4 TCRβ chain of SEQ ID NOs: 5 and 7 respectively, or a TCR comprising the AFP TCRα chain and AFP TCRβ chain of SEQ ID NOs: 22 and 23, respectively.

It may be desirable to provide heterologous TCRs that are mutated or modified relative to the progenitor TCR and have an equal, equivalent or higher affinity and/or an equal, equivalent or slower off-rate for the peptide-HLA complex than the progenitor TCR. The heterologous TCR may have more than one mutation present in the alpha chain variable domain and/or the beta chain variable domain relative to the progenitor TCR and may be denoted, “engineered TCR” or “mutant TCR”. These mutation(s) may improve the binding affinity and/or specificity and/or selectivity and/or avidity for target AFP or MAGE A4 or peptide antigens thereof. In certain embodiments, there are 1, 2, 3, 4, 5, 6, 7 or 8 mutations in alpha chain variable domain, for example 4 or 8 mutations, and/or 1, 2, 3, 4 or 5 mutations in the beta chain variable domain, for example 5 mutations. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 9 (MAGE A4) or amino acids 1-112 of SEQ ID NO: 22 (AFP). In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 10 (MAGE A4) or amino acids 1-112 of SEQ ID NO: 23 (AFP).

The heterologous TCR, (e.g. MAGE A4 TCR), may comprise a TCR in which, the alpha chain variable domain comprises SEQ ID NO: 9 or the amino acid sequence of amino acid residues 1-136 of SEQ ID NO:5 or an amino acid sequence in which amino acid residues 1-47, 54-70, 77-110 and 126-136 thereof have at least 70%, 75%, 80%, 85%, 90%, 95%, 96%, 97%, 98% or 99% identity to the sequence of amino acid residues 1-47, 54-70, 77-110 and 126-136 respectively of SEQ ID NO:9 and/or in which amino acid residues 48-53, 71-76 and 111-125, CDR 1, CDR 2, CDR 3 respectively, have at least 70%, 75%, 80%, 85%, 90%, 95%, 96%, 97%, 98% or 99% identity to the sequence of amino acid residues 48-53, 71-76 and 111-125, CDR 1, CDR 2, CDR 3, respectively of SEQ ID NO:9.

The heterologous TCR may comprise a TCR in which, in the alpha chain variable domain, the sequence of:
(i) amino acid residues 1-47 thereof may have (a) at least 70%, 75%, 80%, 85%, 90% or 95% identity to the sequence of amino acid residues 1-47 of SEQ ID NO:9 or (b) may have one, two or three amino acid residues inserted or deleted relative to residues 1-47 of SEQ ID NO:9,
(ii) amino acid residues 48-53 are VSPFSN, CDR 1, SEQ ID NO:11 or amino acids 48-53 of SEQ ID NO:9,
(iii) amino acid residues 54-70 thereof may have (a) at least 70%, 75%, 80%, 85%, 90% or 95% identity to the sequence of amino acid residues 54-70 of SEQ ID NO: 9 or (b) may have one, two or three amino acid residues inserted or deleted relative to the sequence of amino acid residues 54-70 of SEQ ID NO: 9,
(iv) amino acid residues 71-76 may be LTFSEN, CDR 2, SEQ ID NO: 12 or amino acids 71-76 of SEQ ID NO:9,
(v) amino acid residues 77-110 thereof may have at least 70%, 75%, 80%, 85%, 90% or 95% identity to the sequence of amino acid residues 77-110 of SEQ ID NO:9 or may have one, two or three insertions, deletions or substitutions relative to the sequence of amino acid residues 77-110 of SEQ ID NO:9,
(vi) amino acids 111-125 may be CVVSGGTDSWGKLQF, CDR 3, SEQ ID NO: 13 or amino acids 111-125 of SEQ ID NO:9; and/or
(vii) amino acid residues 126-136 thereof may have at least 70%, 75%, 80%, 85%, 90% or 95% identity to the sequence of amino acid residues 126-136 of SEQ ID NO: 9 or may have one, two or three insertions, deletions or substitutions relative to the sequence of amino acid residues 126-136 of SEQ ID NO:9.

The heterologous TCR, (e.g. MAGE A4 TCR), may comprise a TCR in which, in the beta chain variable domain comprises the amino acid sequence of SEQ ID NO: 10, or an amino acid sequence in which amino acid residues 1-45, 51-67, 74-109, 124-133 thereof have at least 70%, 75%, 80%, 85%, 90% or 95% identity to the sequence of amino acid residues 1-45, 51-67, 74-109, 124-133 respectively of SEQ ID NO: 10 and in which amino acid residues 46-50, 68-73 and 110-123 have at least 70%, 75%, 80%, 85%, 90% or 95% identity to the sequence of amino acid residues 46-50, 68-73 and 110-123, CDR 1, CDR 2, CDR 3, respectively of SEQ ID NO:10.

The heterologous TCR may comprise a TCR in which, in the beta chain variable domain, the sequence of:
(i) amino acid residues 1-45 thereof may have (a) at least 70%, 75%, 80%, 85%, 90% or 95% identity to the sequence of amino acid residues 1-45 of SEQ ID NO: 10 or (b) may have one, two or three amino acid residues inserted or deleted relative to residues 1-45 of SEQ ID NO: 10,
(ii) amino acid residues 46-50 is KGHDR, CDR 1, SEQ ID NO:14 or amino acids 46-50 of SEQ ID NO: 10,
(iii) amino acid residues 51-67 thereof may have (a) at least 70%, 75%, 80%, 85%, 90% or 95% identity to the sequence of amino acid residues 51-67 of SEQ ID NO: 10 or (b) may have one, two or three amino acid residues inserted or deleted relative to the sequence of amino acid residues 51-67 of SEQ ID NO: 10,
(iv) amino acid residues 68-73 may be SFDVKD, CDR 2, SEQ ID NO: 15 or amino acids 68-73 of SEQ ID NO: 10,
(v) amino acid residues 74-109 thereof may have at least 70%, 75%, 80%, 85%, 90% or 95% identity to the sequence of amino acid residues 74-109 of SEQ ID NO: 10 or may have one, two or three insertions, deletions or substitutions relative to the sequence of amino acid residues 74-109 of SEQ ID NO: 10;
(vi) amino acids 110-123 may be CATSGQGAYEEQFF, CDR 3, SEQ ID NO: 16 or amino acids 110-123 of SEQ ID NO: 10, and/or
(vii) amino acid residues 124-133 thereof may have at least 70%, 75%, 80%, 85%, 90% or 95% identity to the sequence of amino acid residues 124-133 of SEQ ID NO: 10 or may have one, two or three insertions, deletions or substitutions relative to the sequence of amino acid residues 124-133 of SEQ ID NO:10.

The heterologous TCR may comprise a TCR which comprises an alpha chain variable domain of SEQ ID NO: 9 and/or a beta chain variable domain of SEQ ID NO: 10. The TCR may comprise a TCR which comprises an alpha chain of SEQ ID NO: 5 and/or a beta chain of SEQ ID NO: 7.

### AFP TCR CDRs and Frameworks

The heterologous TCR, (e.g. AFP TCR), may comprise a TCR in which, the alpha chain variable domain comprises the amino acid sequence of amino acid residues 1-112 of SEQ ID NO: 22, or an amino acid sequence in which amino acid residues 1-26, 33-49, 56-89 and 102-112 thereof have at least 70%, 75%, 80%, 85%, 90%, 95%, 96%, 97%, 98% or 99% identity to the sequence of amino acid residues 1-26, 33-49, 56-89 and 102-112, respectively of SEQ ID NO:22 and/or in which amino acid residues 27-32, 50-55, 90-101, CDR 1, CDR 2, CDR 3 respectively, have at least 70%, 75%, 80%, 85%, 90%, 95%, 96%, 97%, 98% or 99% identity to the sequence of amino acid residues 27-32, 50-55, 90-101, CDR 1, CDR 2, CDR 3, respectively of SEQ ID NO:22.

The heterologous TCR, (e.g. AFP TCR), may comprise a TCR in which, in the alpha chain variable domain, the sequence of:
(i) amino acid residues 1-26 thereof may have (a) at least 70%, 75%, 80%, 85%, 90% or 95% identity to the sequence of amino acid residues 1-26 of SEQ ID NO:22 or (b) may have one, two or three amino acid residues inserted or deleted relative to residues 1-26 of SEQ ID NO:22,
(ii) amino acid residues 27-32 is DRGSQS, αCDR 1, SEQ ID NO:26 or amino acids 27-32 of SEQ ID NO:22, or DRGSQA, SEQ ID NO:32,
(iii) amino acid residues 33-49 thereof may have (a) at least 70%, 75%, 80%, 85%, 90% or 95% identity to the sequence of amino acid residues 33-49 of SEQ ID NO: 22 or (b) may have one, two or three amino acid residues inserted or deleted relative to the sequence of amino acid residues 33-49 of SEQ ID NO: 22,
(iv) amino acid residues 50-55 may be IYSNGD, αCDR 2, SEQ ID NO:27 or amino acids 50-55 of SEQ ID NO:22,
(v) amino acid residues 56-89 thereof may have at least 70%, 75%, 80%, 85%, 90% or 95% identity to the sequence of amino acid residues 56-89 of SEQ ID NO:22 or may have one, two or three insertions, deletions or substitutions relative to the sequence of amino acid residues 56-89 of SEQ ID NO:22,
(vi) amino acids 90-100 may be AVNSDSGYALN or amino acids 90-100 of SEQ ID NO: 22,
(vii) amino acids 90-101 may be AVNSDSGYALNF, αCDR 3, SEQ ID NO:28 or amino acids 90-101 of SEQ ID NO:22,
(viii) amino acid residues 102-112 thereof may have at least 70%, 75%, 80%, 85%, 90% or 95% identity to the sequence of amino acid residues 102-112 of SEQ ID NO: 22 or may have one, two or three insertions, deletions or substitutions relative to the sequence of amino acid residues 102-112 of SEQ ID NO: 22.

The TCR, (e.g. AFP TCR), may comprise a TCR in which, in the beta chain variable domain comprises the amino acid sequence of amino acid residues 1-112 of SEQ ID NO: 23, or an amino acid sequence in which amino acid residues 1-26, 32-48, 55-91, 103-112 thereof have at least 70%, 75%, 80%, 85%, 90% or 95% identity to the sequence of amino acid residues 1-26, 32-48, 55-91, 103-112 respectively of SEQ ID NO: 23 and in which amino acid residues 27-31, 49-54 and 92-102 have at least 70%, 75%, 80%, 85%, 90% or 95% identity to the sequence of amino acid residues 27-31, 49-54 and 92-102, βCDR 1, βCDR 2, βCDR 3, respectively of SEQ ID NO: 23.

The TCR may comprise a TCR in which, in the beta chain variable domain, the sequence of:
(i) amino acid residues 1-26 thereof may have (a) at least 70%, 75%, 80%, 85%, 90% or 95% identity to the sequence of amino acid residues 1-26 of SEQ ID NO: 23 or (b) may have one, two or three amino acid residues inserted or deleted relative to residues 1-26 of SEQ ID NO: 23,
(ii) amino acid residues 27-31 is SGDLS, βCDR 1, SEQ ID NO:29 or amino acids 27-31 of SEQ ID NO: 23,
(iii) amino acid residues 32-48 thereof may have (a) at least 70%, 75%, 80%, 85%, 90% or 95% identity to the sequence of amino acid residues 32-48 of SEQ ID NO: 23 or (b) may have one, two or three amino acid residues inserted or deleted relative to the sequence of amino acid residues 32-48 of SEQ ID NO: 23,
(iv) amino acid residues 49-54 may be YYNGEE, βCDR 2, SEQ ID NO: 30 or amino acids 49-54 of SEQ ID NO: 23,
(v) amino acid residues 55-91 thereof may have at least 70%, 75%, 80%, 85%, 90% or 95% identity to the sequence of amino acid residues 55-91 of SEQ ID NO: 23 or may have one, two or three insertions, deletions or substitutions relative to the sequence of amino acid residues 55-91 of SEQ ID NO: 23
(vi) amino acids 92-102 may be ASSLGGESEQY, βCDR 3, SEQ ID NO: 31 or amino acids 92-102 of SEQ ID NO: 23,
(vii) amino acid residues 103-112 thereof may have at least 70%, 75%, 80%, 85%, 90% or 95% identity to the sequence of amino acid residues 103-112 of SEQ ID NO: 23 or may have one, two or three insertions, deletions or substitutions relative to the sequence of amino acid residues 103-112 of SEQ ID NO: 23.

The heterologous TCR may comprise a TCR in which the alpha chain comprises amino acid residues of SEQ ID No: 41, and the beta chain variable domain comprises amino acid residues of SEQ ID No: 23 or SEQ ID NO: 42.

### Mutant AFP TCRs

In some embodiments, a heterologous TCR may be mutated relative to the parental AFP TCR, (in which the alpha chain comprises amino acid residues of SEQ ID No: 22, and the beta chain variable domain comprises amino acid residues of SEQ ID No: 23) or an AFP TCR in which in which the alpha chain comprises amino acid residues of SEQ ID No: 41, and the beta chain variable domain comprises amino acid residues of SEQ ID No: 23 or SEQ ID NO: 42.

Accordingly, such a mutated heterologous TCR may comprise an
a. αCDR1 having the sequence DRGSQA, SEQ ID NO:32,
b. αCDR3 having the sequence AVNSDSSYALNF, SEQ ID NO:33,
c. αCDR3 having the sequence AVNSDSGVALNF, SEQ ID NO:34,
d. αCDR1 having the sequence DRGSQA, SEQ ID NO:32 and αCDR3 having the sequence AVNSDSGVALNF, SEQ ID NO:34,
e. αCDR3 having the sequence AVNSQSGYALNF, SEQ ID NO: 35,
f. αCDR3 having the sequence AVNSQSGYSLNF, SEQ ID NO: 36,
g. αCDR3 having the sequence AVNSQSSYALNF, SEQ ID NO: 40,
h. αCDR1 having the sequence DRGSQA, SEQ ID NO:32 and αCDR3 having the sequence AVNSQSGYALNF, SEQ ID NO: 35,
i. αCDR3 having the sequence AVNSQSGVALNF, SEQ ID NO: 36,
j. αCDR3 having the sequence AVNSQNGYALNF, SEQ ID NO: 37,
k. αCDR 1 having the sequence DRGSFS, SEQ ID NO: 38,
l. αCDR1 having the sequence DRGSYS, SEQ ID NO: 39,
m. αCDR1 having the sequence DRGSYS, SEQ ID NO: 39 and αCDR3 having the sequence AVNSDSSYALNF SEQ ID NO: 33,
n. αCDR1 having the sequence DRGSYS, SEQ ID NO: 39 and αCDR3 having the sequence AVNSDSGVALNF SEQ ID NO: 34; or
o. αCDR1 having the sequence DRGSYS, SEQ ID NO: 39 and αCDR3 having the sequence AVNSQSGYALNF, SEQ ID NO: 35.

The heterologous TCR, or heterologous mutated TCR, may comprise an alpha chain variable domain that includes a mutation in one or more of the amino acids corresponding to: 31Q, 32S, 94D, 95S, 96G, 97Y, and 98A, with reference to the numbering shown in SEQ ID No: 22. For example, the alpha chain variable domain may have one or more of the following mutations: Q31F/Y, S32A, D94Q, S95N, G96S, Y97V, A98S, according to the numbering shown in SEQ ID No: 22.

The heterologous TCR may comprise a TCR alpha chain variable domain of SEQ ID NO: 22 but that includes a mutation in one or more of the amino acids corresponding to: 31Q, 32S, 94D, 95S, 96G, 97Y, and 98A, for example one or more of Q31F/Y, S32A, D94Q, S95N, G96S, Y97V, A98S, according to the numbering shown in SEQ ID No: 22, and/or a beta chain variable domain comprising D1 to T112 of SEQ ID NO: 23 or SEQ ID NO: 42.

The heterologous TCR may comprise a TCR alpha chain variable domain and a TCR beta chain variable domain, wherein:
(i) the alpha chain variable domain comprises CDRs having the sequences DRGSQA (αCDR1), SEQ ID NO:32 or amino acids 27-32 of SEQ ID NO:41, IYSNGD (αCDR2), SEQ ID NO:27 or amino acids 50-55 of SEQ ID NO:22, and AVNSQSGYALNF (αCDR3), SEQ ID NO:35 or amino acids 90-101 of SEQ ID NO: 41, and
(ii) the beta chain variable domain comprises CDRs having the sequences SGDLS (βCDR1), SEQ ID NO:29 or amino acids 27-31 of SEQ ID NO:23, YYNGEE (βCDR2), SEQ ID NO:30 or amino acids 49-54 of SEQ ID NO:23, and ASSLGGESEQY (βCDR3), SEQ ID NO:31 or amino acids 92-102 of SEQ ID NO:23.

The heterologous TCR may comprise a TCR in which the alpha chain variable domain comprises amino acid residues 1-112 of SEQ ID No: 41, and the beta chain variable domain comprises amino acid residues 1-112 of SEQ ID No: 33 or SEQ ID NO:42.

The heterologous TCR may comprise a TCR in which the alpha chain comprises amino acid residues of SEQ ID No: 41, and the beta chain variable domain comprises amino acid residues 1-112 of SEQ ID No: 33 or SEQ ID NO:42.

### TCR Variants

Amino acid and nucleotide sequence identity is generally defined with reference to the algorithm GAP (GCG Wisconsin Package™, Accelrys, San Diego CA). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST, psiBLAST or TBLASTN (which use the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), generally employing default parameters.

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, up to 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue, or an aromatic residue may be replaced by another aromatic residue. Conservative substitutions may, for example, be between amino acids within the following groups:
alanine and glycine;
glutamic acid, aspartic acid, glutamine, and asparagine arginine and lysine;
asparagine, glutamine, glutamic acid and aspartic acid
isoleucine, leucine and valine;
phenylalanine, tyrosine, and tryptophan; and
serine, threonine, and cysteine.

### The CD8α Co-Receptor

A modified iPSC or haemogenic lineage cell, such as a T cell, comprising at least one heterologous nucleic acid sequence encoding a heterologous TCR integrated into a locus in the cell genome and/or expressing or presenting a heterologous TCR described herein may further express or present a heterologous co-receptor (e.g., the cell is transduced with or engineered to comprise for example by gene knock-in, a nucleic acid encoding the co-receptor, e.g. CD8 co-receptor).

The heterologous co-receptor may be a CD8 co-receptor. The CD8 co-receptor may comprise a dimer or pair of CD8 chains which comprises a CD8-α and CD8-β chain or a CD8-α and CD8-α chain. Preferably, the CD8 co-receptor is a CD8αα co-receptor comprising a CD8-α and CD8-α chain. A CD8α co-receptor may comprise the amino acid sequence of at least 80% identity to SEQ ID NO: 3, SEQ ID NO: 3 or a variant thereof. The CD8α co-receptor may be a homodimer.

Preferably, the CD8 co-receptor binds to class 1 MHCs and potentiates TCR signaling. The CD8 co-receptor may comprise the reference amino acid sequence of SEQ ID NO: 3 or may be a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence SEQ ID NO: 3. The CD8 co-receptor may be encoded by the reference nucleotide sequence of SEQ ID NO: 4 or may be a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence SEQ ID NO: 4.

The heterologous CD8 co-receptor may comprise a CD8 co-receptor in which, in the Ig like V-type domain comprises CDRs having the sequence;
(i) VLLSNPTSG, CDR1, SEQ ID NO: 17, or amino acids 45-53 of SEQ ID NO: 3,
(ii) YLSQNKPK, CDR2, SEQ ID NO: 18 or amino acids 72-79 of SEQ ID NO: 3,
(iii) LSNSIM, CDR3, SEQ ID NO: 19 or amino acids 80-117 of SEQ ID NO: 3,
or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto respectively.

The heterologous CD8 co-receptor may comprise a CD8 co-receptor which comprises or in which, in the Ig like V-type domain comprises, residues 22-135 of the amino acid sequence of SEQ ID No:3, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, CDR 1, CDR 2, CDR 3, respectively of SEQ ID No:3 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID NO:3.

The CD8 co-receptor may comprise a CD8 co-receptor in which,
(i) amino acid residues 22-44 thereof may have (a) at least 70%, 75%, 80%, 85%, 90% or 95% identity to the sequence of amino acid residues 22-44 of SEQ ID NO:3 or (b) may have one, two or three amino acid residues inserted or deleted relative to residues 22-44 of SEQ ID NO:3,
(ii) amino acid residues 45-53 is VLLSNPTSG, SEQ ID NO:17, CDR1, or amino acids 45-53 of SEQ ID NO:3,
(iii) amino acid residues 54-71 thereof may have (a) at least 70%, 75%, 80%, 85%, 90% or 95% identity to the sequence of amino acid residues 54-71 of SEQ ID NO:3 or (b) may have one, two or three amino acid residues inserted or deleted relative to the sequence of amino acid residues 54-71 of SEQ ID NO:3,
(iv) amino acid residues 72-79 may be YLSQNKPK, CDR2, SEQ ID NO: 18 or amino acids 72-79 of SEQ ID NO:3,
(v) amino acid residues 80-117 thereof may have at least 70%, 75%, 80%, 85%, 90% or 95% identity to the sequence of amino acid residues 80-117 of SEQ ID NO:3 or may have one, two or three insertions, deletions or substitutions relative to the sequence of amino acid residues 80-117 of SEQ ID NO:3;
(vi) amino acids 118-123 may be LSNSIM, CDR3, SEQ ID NO: 19 or amino acids 80-117 of SEQ ID NO:3,
(vii) amino acid residues 124-135 thereof may have at least 70%, 75%, 80%, 85%, 90% or 95% identity to the sequence of amino acid residues 124-135 of SEQ ID NO:3 or may have one, two or three insertions, deletions or substitutions relative to the sequence of amino acid residues 124-135 of SEQ ID NO:3.

The modified induced pluripotent stem cells iPSC or haemogenic lineage cells, preferably T cells, that comprise a nucleic acid encoding the heterologous CD8 co-receptor and/or which express heterologous CD8 co-receptor may demonstrate improved affinity and/or avidity and/or improved T-cell activation, as determinable by the assays disclosed herein, towards or on stimulation by antigenic peptide, tumour or cancer antigen optionally when presented on HLA relative to modified cells (e.g. induced pluripotent stem cells iPSC or haemogenic lineage cells, preferably T cells) that do not express heterologous CD8 co-receptor.

The heterologous CD8 of modified cells may interact or bind specifically to an MHC. The MHC may be class I or class II, preferably class I major histocompatibility complex (MHC), HLA-I molecule or the MHC class I HLA-A/B2M dimer. Preferably the CD8-α interacts with the α3 portion of the Class I MHC (between residues 223 and 229), preferably via the IgV-like domain of CD8. The heterologous CD8 may improve TCR binding of the cells to the HLA and/or antigenic peptide bound or presented by HLA pMHCI or pHLA, optionally on the surface of antigen presenting cell, dendritic cell and/or tumour or cancer cell, tumour or cancer tissue compared to cells lacking the heterologous CD8.

The heterologous CD8 can improve or increase the off-rate (koff) of the cell (TCR)/peptide-major histocompatibility complex class I (pMHCI) interaction of the modified iPSCs or haemogenic lineage cells, preferably T cells, and hence its half-life, optionally on the surface of antigen presenting cell, dendritic cell and/or tumour or cancer cell, or tumour or cancer tissue compared to cells (iPSC or haemogenic lineage cells, preferably T cells) lacking the heterologous CD8, and thereby may also provide improved ligation affinity and/or avidity. The heterologous CD8 can improve organizing the TCR on the stem cells iPSC or haemogenic lineage cells surface to enable cooperativity in pHLA binding and may provide improved therapeutic avidity. Accordingly, the heterologous CD8 co-receptor modified induced pluripotent stem cells iPSC or haemogenic lineage cells, preferably T cells, may bind or interact with LCK (lymphocyte-specific protein tyrosine kinase) in a zinc-dependent manner leading to activation of transcription factors like NFAT, NF-κB, and AP-1.

The modified induced pluripotent stem cells iPSC or haemogenic lineage cells, preferably T cells, may have an improved or increased expression of CD40L, cytokine production, cytotoxic activity, induction of dendritic cell maturation or induction of dendritic cell cytokine production, optionally in response to cancer and/or tumour antigen or peptide antigen thereof optionally as presented by tumour of cancer cell or tissue, in comparison to iPSC or haemogenic lineage cells, preferably T cells, lacking the heterologous CD8 co-receptor.

### Co-Stimulatory Ligand

The modified pluripotent stem cell iPSC or haemogenic lineage cells, such as T cells, may further comprise (e.g. express or present) an exogenous or heterologous or recombinant co-stimulatory ligand (e.g., the cell is transduced with or engineered to comprise for example by gene knock-in, a nucleic acid encoding the co-stimulatory ligand) at least one co-stimulatory ligand, optionally one, two, three or four. The modified cell or cells may co-express the heterologous TCR and the at least one exogenous co-stimulatory ligand. The interaction between the heterologous TCR and at the least one exogenous co-stimulatory ligand may provide a non-antigen-specific signal and activation of the cell.

Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. TNF superfamily members include, but are not limited to, nerve growth factor (NGF), CD40L (CD40L)/CDI54, CD137L/4-1BBL, TNF-alpha, CD134L/OX40L/CD252, CD27L/CD70, Fas ligand (FasL), CD30L/CD153, tumour necrosis factor beta (TNFP)/lymphotoxin-alpha (LTa), lymphotoxin-beta (TTb), CD257/B cell-activating factor (BAFF)/Blys/THANK/TalI-I, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof. The at least one exogenous or recombinant co-stimulatory ligand can be 4-1BBL or CD80, preferably, the at least one exogenous or recombinant co-stimulatory ligand is 4-1BBL.

The modified pluripotent stem cell iPSC or haemogenic lineage cells, such as T cells, may comprise two exogenous or recombinant co-stimulatory ligands, preferably the two exogenous or recombinant co-stimulatory ligands are 4-1BBL and CD80.

The modified cells may comprise an exogenous or a recombinant (e.g., the cell is transduced with or engineered to comprise for example by gene knock-in, a nucleic acid encoding) at least one construct which overcomes the immunosuppressive tumour microenvironment. Such constructs can be, but are not limited to, cyclic AMP phosphodiesterases and dominant-negative transforming growth factor beta (TGFbeta) receptor II. The modified stem cells iPSC or haemogenic lineage cells, for example a modified T cell or a population of modified T cells, may be engineered to release cytokines which have a positive effect on the cytolytic activity of said cells. Such cytokines include, but are not limited to interleukin-7, interleukin-15 and interleukin-21.

### Cell Function and Activation

The present invention provides a modified iPSC or haemogenic lineage cell according to the invention, preferably a T cell, wherein the binding of the iPSC or haemogenic lineage cell and/or heterologous TCR to the antigen or peptide antigen, e.g. cancer and/or tumour antigen or peptide antigen thereof or a cancer and/or tumour cell or tissue expressing or presenting the cancer and/or tumour antigen or peptide antigen thereof as herein described, can induce activation and/or function of the iPSC or haemogenic lineage cell, optionally as determined by any one or more of;
(a) up-regulation of T cell activation markers, for example either CD69 and/or CD25 on CD3+ cells,
(b) up-regulation of cytokine production, for example any one or more of IFN gamma, IL-2 or Granzyme B,
(c) induced cell cytotoxic activity in the presence of the antigen or antigen peptide,
(d) ability to kill tumour cells presenting the antigen or antigen peptide,
(e) increased iPSC or haemogenic lineage cell secretion of cytokines and/or interferon,
(f) increased iPSC or haemogenic lineage cell proliferation, for example as judged by cell markers (for example as judged by Ki67 expression level),
(g) increased iPSC or haemogenic lineage cell antigen responsiveness,
(h) increased iPSC or haemogenic lineage cell target cell killing,
(i) increased iPSC or haemogenic lineage cell CD28 signaling,
(j) increased iPSC or haemogenic lineage cell ability to infiltrate tumour,
(k) increased ability of iPSC or haemogenic lineage cell to recognise and bind to dendritic cell presented antigen.
(l) increased iPSC or haemogenic lineage cell ability to resist T-cell repressive factors as determined for example by a reduced repressive response to T-regs, Myeloid derived suppressor cells (MDSCs), PD-L1 protein expression, or serum cytokine levels selected from CCL3, IL8, IL1β, CXCL10, or sIL2Rα or levels of inhibitory receptors, selected from PD-1, CTLA-4, TIM-3, LAG-3, BTLA or TIGIT, or
(m) increase in level of interferon-γ, interleukin-6, interleukin-10, cytokine production, such as IL-2, TNF-α, IFN-γ and granzyme B by the iPSC or haemogenic lineage cell.

Preferably, the iPSC or haemogenic lineage cell of (a) to (m) is a T cell.

### TCR+ Cell Persistence

The present invention also provides a modified iPSC or haemogenic lineage cell according to the invention, preferably a T cell, which demonstrates improved TCR+ cell persistence in culture or subject samples for example, as determined by the measurement of the persistence of modified pluripotent stem cell iPSC or haemogenic lineage cells expressing or presenting a heterologous T-cell receptor (TCR) as herein described. Persistence of the infused engineered and modified pluripotent stem cell iPSC or haemogenic lineage cells is correlated with therapeutic effect and is also a long-term safety measure. Cell persistence can be determined by qPCR or flow cytometry (FCM). For example, the quantitation of TCR+ (e.g. AFP TCR or MAGE-A4 TCR) cells by PCR of coding gene from DNA. Alternatively, quantification of T cell phenotype and activity associated with the heterologous TCR may be determined by a range of assays, for example:
Phenotype analysis for determination of T-cell lineages in cell product/culture or in the subject blood pre and post infusion.
Quantitation of the senescence and activation status of T-cells in cell product/culture or from subject samples.
Quantitation of soluble factors reflecting in vivo function of TCR+ T cells,

### Nucleic Acid Construct or Vector

The present invention also provides a nucleic acid construct or vector comprising a nucleic acid region encoding the heterologous TCR according to the invention and at least one homology region comprising a nucleic acid region homologous to a nucleic acid region at the locus in the cell genome for integration of the nucleic acid region encoding the heterologous TCR. Preferably the locus is as herein before described.

Preferably the locus is or is in a gene encoding a member of the elongation factor complex, preferably a translation elongation factor, preferably eukaryotic translation elongation factor 1 alpha 1 optionally wherein the locus is at exon 8 of the eukaryotic translation elongation factor 1 alpha 1 (EEF1A1) gene, optionally before the TAG stop codon or immediately adjacent to and/or before the TAG stop codon.

The nucleic acid encoding the heterologous TCR may comprise a coding sequence of a TCRα and/or TCRβ chain, for example as described herein, optionally with an intervening nucleic acid sequence encoding a peptide comprising an enzymatic cleavage site and/or nucleic acid sequence which mediates ribosome-skipping, preferably wherein the nucleic acid sequence encoding a peptide comprising an enzymatic cleavage site encodes a furin cleavage site, preferably RAKR, preferably wherein the nucleic acid sequence which mediates ribosome-skipping is a ribosome skip sequence, preferably T2A and/or P2A skip sequence.

The construct or vector can comprise a left hand and/or a right hand homology region each homologous to a nucleic acid region at the locus in the cell genome for integration of the nucleic acid region encoding the heterologous TCR, optionally which flank opposite sides of the integration site.

Accordingly, the construct or vector may further comprise any one or more of:
(a) a recombination target sequence, preferably loxP (locus of X-over P1) sequence, for example the loxP sequence of SEQ ID No: 48,
(b) an expressible selection marker sequence, preferably an antibiotic resistance gene, optionally a neomycin resistance gene, preferably constitutively expressed from a promoter for example from an EF1A promoter, such as the EF1A promoter sequence of SEQ ID No: 47.

The construct or vector may comprise a nucleotide sequence encoding the heterologous TCR comprising the sequence SEQ ID No: 45, homology regions comprising the sequences SEQ ID No: 43 and SEQ ID No: 44, and optionally a recombination target sequence comprising SEQ ID No: 48 and/or an expressible selection marker sequence comprising SEQ ID No: 47, SEQ ID No: 49 or SEQ ID No: 56. In some embodiments, the construct or vector may comprise the nucleotide sequence of SEQ ID No: 57.

### Production of Cell Populations

The present invention further provides a process of producing a modified iPSC or haemogenic lineage cell, such as a T cell, according to the invention comprising introducing the nucleic acid construct or vector according to the invention into an unmodified iPSC or haemogenic lineage cell, optionally as herein described, under conditions to permit integration of the nucleic acid sequence encoding a heterologous T-cell receptor (TCR) at or into a locus in the cell genome and optionally isolating the modified iPSC or haemogenic lineage cell. The process may be achieved using known genome editing methodologies such as those which employ AAV, Transcription activator-like effector nucleases (TALEN), CRISPR, Zinc finger nucleases, or engineered meganucleases.

Preferred embodiments provide processes relating to the introduction of the nucleic acid construct or vector according to the invention into an unmodified iPSC to produce a modified iPSC, optionally as herein described, and the differentiation of the modified iPSC into a T cell. For example, a method of producing a population of T cells may comprise;
(i) introducing a heterologous nucleic acid encoding a T-cell receptor at or into a locus in the cell genome of a population of iPSCs, and
(ii) differentiating the population of iPSCs into T cells.

Other embodiments provide processes relating to the production of a modified immune cell, such as a T cell. For example, a method of producing a population of T cells may comprise:
(i) optionally, differentiating a population of iPSCs into T cells (or other immune cells), or acquiring a population of T cells (or other immune cells) from a different source; and
(i) introducing a heterologous nucleic acid encoding a T-cell receptor at or into a locus in the cell genome of the T cells (or other immune cells).

Preferably, the locus in the cell genome is the eukaryotic translation elongation factor 1 alpha 1 (EEF1A1) gene.

In some embodiments, iPSCs for use in the methods described herein may be RAG inactivated iPSCs (RAG−/−iPSCs), for example RAG1 and/or RAG2 inactivated iPSCs.

The population of induced pluripotent stem cells (iPSCs) may be differentiated into T cells by differentiating the population of iPSCs into haemogenic lineage cells and differentiating and/or maturing the haemogenic lineage cells into T cells. For example, a population of induced pluripotent stem cells (iPSCs) may be differentiated into T cells by a method comprising;
(i) differentiating a population of induced pluripotent stem cells (iPSCs) into mesoderm cells
(ii) differentiating the mesoderm cells (MCs) to produce a population of haemogenic endothelial cells (HECs)
(iii) differentiating the HECs into a population of haematopoietic progenitor cells (HPCs);
(iv) differentiating the population of HPCs into progenitor T cells; and
(v) maturing the progenitor T cells to produce a population of double positive CD4+ CD8+ T cells.

The method may further comprise;
(vi) activating and expanding the T cells to produce a population of single positive CD8+ T cells or a population of single positive CD4+ T cells.

Differentiation and maturation of the cell populations in the steps of the methods described herein is induced by culturing the cells in a culture medium supplemented with a set of differentiation factors. The set of differentiation factors that is listed for each culture medium is preferably exhaustive and medium may be devoid of other differentiation factors. In preferred embodiments, the culture media are chemically defined media. For example, a culture medium may consist of a chemically defined nutrient medium that is supplemented with an effective amount of one or more differentiation factors, as described below. A chemically defined nutrient medium may comprise a basal medium that is supplemented with one or more serum-free culture medium supplements.

Differentiation factors are factors which modulate, for example promote or inhibit, a signaling pathway which mediates differentiation in a mammalian cell. Differentiation factors may include growth factors, cytokines and small molecules which modulate one or more of the Activin/Nodal, FGF, Wnt or BMP signaling pathways. Examples of differentiation factors include Activin/Nodal, FGFs, BMPs, retinoic acid, vascular endothelial growth factor (VEGF), stem cell factor (SCF), TGFβ ligands, GDFs, LIF, Interleukins, GSK-3 inhibitors and phosphatidylinositol 3-kinase (PI3K) inhibitors.

Differentiation factors which are used in one or more of the media described herein include TGFβ ligands, such as activin, fibroblast growth factor (FGF), bone morphogenetic protein (BMP), stem cell factor (SCF), vascular endothelial growth factor (VEGF), GSK-3 inhibitors (such as CHIR-99021), interleukins, and hormones, such as IGF-1 and angiotensin II. A differentiation factor may be present in a medium described herein in an amount that is effective to modulate a signaling pathway in cells cultured in the medium.

In some embodiments, a differentiation factor listed above or below may be replaced in a culture medium by a factor that has the same effect (i.e. stimulation or inhibition) on the same signaling pathway. Suitable factors are known in the art and include proteins, nucleic acids, antibodies and small molecules.

The extent of differentiation of the cell population during each step may be determined by monitoring and/or detecting the expression of one or more cell markers in the population of differentiating cells. For example, an increase in the expression of markers characteristic of the more differentiated cell type or a decrease in the expression of markers characteristic of the less differentiated cell type may be determined. The expression of cell markers may be determined by any suitable technique, including immunocytochemistry, immunofluorescence, RT-PCR, immunoblotting, fluorescence activated cell sorting (FACS), and enzymatic analysis. In preferred embodiments, a cell may be said to express a marker if the marker is detectable on the cell surface. For example, a cell which is stated herein not to express a marker may display active transcription and intracellular expression of the marker gene, but detectable levels of the marker may not be present on the surface of the cell.

A population of partially differentiated cells that is produced by a step in the methods described herein may be cultured, maintained or expanded before the next differentiation step. Partially differentiated cells may be expanded by any convenient technique.

After each step, the population of partially differentiated cells which is produced by that step may contain 1% or more, 5% or more, 10% or more or 15% or more partially differentiated cells, following culture in the medium. If required, the population of partially differentiated cells may be purified by any convenient technique, such as MACs or FACS.

Cells may be cultured in a monolayer, in the absence of feeder cells, on a surface or substrate coated with extracellular matrix protein, such as fibronectin, laminin or collagen. Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho WY et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% or 21% Oxygen, 5% Carbon Dioxide. Media is preferably changed every two days and cells allowed to settle by gravity.

Cells may be cultured in a culture vessel. Suitable cell culture vessels are well-known in the art and include culture plates, dishes, flasks, bioreactors, and multi-well plates, for example 6-well, 12-well or 96-well plates.

The culture vessels are preferably treated for tissue culture, for example by coating one or more surfaces of the vessel with an extracellular matrix protein, such as fibronectin, laminin or collagen. Culture vessels may be treated for tissue culture using standard techniques, for example by incubating with a coating solution, as described herein, or may be obtained pre-treated from commercial suppliers.

In a first stage, iPSCs may be differentiated into mesoderm cells by culturing the population of iPSCs under suitable conditions to promote mesodermal differentiation. For example, the iPSCs cells may be cultured sequentially in first, second and third mesoderm induction media to induce differentiation into mesoderm cells.

A suitable first mesoderm induction medium may stimulate SMAD2 and SMAD3 mediated signaling pathways. For example, the first mesoderm induction medium may comprise activin.

A suitable second mesoderm induction medium may (i) stimulate SMAD1, SMAD2, SMAD3, SMAD5 and SMAD9 and/or SMAD1, SMAD2, SMAD3, SMAD5 and SMAD9 mediated signaling pathways and (ii) have fibroblast growth factor (FGF) activity. For example, the second mesoderm induction medium may comprise activin, preferably activin A, BMP, preferably BMP4 and FGF, preferably bFGF.

A suitable third mesoderm induction medium may (i) stimulate SMAD1, SMAD2, SMAD3, SMAD5 and SMAD9 and/or SMAD1, SMAD2, SMAD3, SMAD5 and SMAD9 mediated signaling pathways (ii) have fibroblast growth factor (FGF) activity and (iii) inhibit glycogen synthase kinase 3β. For example, the third mesoderm induction medium may comprise activin, preferably activin A, BMP, preferably BMP4, FGF, preferably bFGF, and a GSK3 inhibitor, preferably CHIR99021.

The first, second and third mesoderm induction media may be devoid of differentiation factors other than the differentiation factors set out above.

SMAD2 and SMAD3 mediated intracellular signaling pathways may be stimulated by the first, second and third mesoderm induction media through the presence in the media of a first TGFβ ligand. The first TGFβ ligand may be Activin. Activin (Activin A: NCBI Gene ID: 3624 nucleic acid reference sequence NM_002192.2 GI: 62953137, amino acid reference sequence NP_002183.1 GI: 4504699) is a dimeric polypeptide which exerts a range of cellular effects via stimulation of the Activin/Nodal pathway (Vallier et al., Cell Science 118:4495-4509 (2005)). Activin is readily available from commercial sources (e.g. Stemgent Inc. MA USA; Miltenyi Biotec Gmbh, DE). Conveniently, the concentration of Activin in a medium described herein may be from 1 to 100 ng/ml, for example any of about 2, 3, 4, 5, 6, 7, 8, 9, 10, 12, 15, 17, 20, 25, 30, 35, 40, 45 or 50, 55, 60, 65, 70, 75, 80, 85, 90 or 95 ng/ml, preferably about 5 to 50 ng/ml.

The fibroblast growth factor (FGF) activity of the second and third mesoderm induction media may be provided by the presence of fibroblast growth factor (FGF) in the media. Fibroblast growth factor (FGF) is a protein factor which stimulates cellular growth, proliferation and cellular differentiation by binding to a fibroblast growth factor receptor (FGFR). Suitable fibroblast growth factors include any member of the FGF family, for example any one of FGF1 to FGF14 and FGF15 to FGF23. Preferably, the FGF is FGF2 (also known as bFGF, NCBI GeneID: 2247, nucleic acid sequence NM_002006.3 GI: 41352694, amino acid sequence NP_001997.4 GI: 41352695); FGF7 (also known as keratinocyte growth factor (or KGF), NCBI GeneID: 2247, nucleic acid sequence NM_002006.3 GI: 41352694, amino acid sequence NP_001997.4 GI: 41352695); or FGF10 (NCBI GeneID: 2247, nucleic acid sequence NM_002006.3 GI: 41352694, amino acid sequence NP_001997.4 GI: 41352695). Most preferably, the fibroblast growth factor is FGF2.

Conveniently, the concentration of FGF, such as FGF2 in a medium described herein may be from 0.5 to 50 ng/ml, for example any of about 0.5, 1, 2, 3, 4, 5, 6, 7, 8, 9, 10, 11, 12, 13, 14, 15, 20, 25, 30, 35, 40, 45, 50 ng/ml, preferably about 5 ng/ml. Fibroblast growth factors, such as FGF2, FGF7 and FGF10, may be produced using routine recombinant techniques or obtained from commercial suppliers (e.g. R&D Systems, Minneapolis, MN; Stemgent Inc, USA; Miltenyi Biotec Gmbh, DE).

SMAD1, SMAD5 and SMAD9 mediated intracellular signaling pathways may be stimulated by the second and third mesoderm induction media through the presence in the media of a second TGFβ ligand.

The second TGFβ ligand may be a Bone Morphogenic Protein (BMP). Bone Morphogenic Proteins (BMPs) bind to Bone Morphogenic Protein Receptors (BMPRs) and stimulate intracellular signaling through pathways mediated by SMAD1, SMAD5 and SMAD9. Suitable Bone Morphogenic Proteins include any member of the BMP family, for example BMP2, BMP3, BMP4, BMP5, BMP6 or BMP7. Preferably the second TGFβ ligand is BMP2(NCBI GeneID: 650, nucleic acid sequence NM_001200.2 GI: 80861484; amino acid sequence NP_001191.1 GI: 4557369) or BMP4 (NCBI GeneID: 652, nucleic acid sequence NM_001202.3 GI: 157276592; amino acid sequence NP_001193.2 GI: 157276593). Suitable BMPs include BMP4. Conveniently, the concentration of a Bone Morphogenic Protein, such as BMP2 or BMP4 in a medium described herein may be from 1 to 500 ng/ml, for example any of about 0.5, 1, 2, 3, 4, 5, 6, 7, 8, 9, 10, 11, 12, 13, 14, 15, 20, 25, 30, 35, 40, 45, 50, 100, 150, 200, 250, 300, 350, 400, 450, 500 ng/ml, preferably about 10 ng/ml. BMPs may be produced using routine recombinant techniques or obtained from commercial suppliers (e.g. R&D, Minneapolis, USA, Stemgent Inc, USA; Miltenyi Biotec Gmbh, DE).

The GSK3β inhibition activity of the third mesoderm induction medium may be provided by the presence of a GSK3β inhibitor in the medium. GSK3β inhibitors inhibit the activity of glycogen synthase kinase 3β (Gene ID 2932: EC2.7.11.26). Preferred inhibitors specifically inhibit the activity of glycogen synthase kinase 3β. Suitable inhibitors include CHIR99021 (6-((2-((4-(2,4-Dichlorophenyl)-5-(4-methyl-1H-imidazol-2-yl) pyrimidin-2-yl)amino)ethyl)amino)nicotinonitrile; Ring D. B. et al., Diabetes, 52:588-595 (2003)) alsterpaullone, kenpaullone, BIO(6-bromoindirubin-3′-oxime (Sato et al Nat Med. 2004 January; 10(1):55-63), SB216763 (3-(2,4-dichlorophenyl)-4-(1-methyl-1H-indol-3-yl)-1H-pyrrole-2,5-dione), Lithium and SB415286 (3-[(3-chloro-4-hydroxyphenyl)amino]-4-(2-nitrophenyl)-1H-pyrrole-2,5-dione; Coghlan et al Chem Biol. 2000 October; 7(10):793-803). In some preferred embodiments, the GSK3β inhibitor is CHIR99021.Suitable glycogen synthase kinase 3β inhibitors may be obtained from commercial suppliers (e.g. Stemgent Inc. MA USA; Cayman Chemical Co. MI USA; Selleckchem, MA USA). For example, the third mesoderm induction medium may contain 0.1 to 100 μM, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 μM, of a GSK3β inhibitor, such as CHIR99021, preferably about 10 μM.

In preferred embodiments, the first, second and third mesoderm induction media are chemically defined media. For example, the first mesoderm induction medium may consist of a chemically defined nutrient medium supplemented with an effective amount of activin, preferably activin A, for example 50 ng/ml activin A; the second mesoderm induction medium may consist of a chemically defined nutrient medium supplemented with an effective amount of activin preferably activin A, for example 5 ng/ml activin A, BMP, preferably BMP4, for example 10 ng/ml BMP4; and FGF, preferably bFGF (FGF2), for example 5 ng/ml bFGF; and the third mesoderm induction medium may consist of a chemically defined nutrient medium supplemented with an effective amount of activin preferably activin A, for example 5 ng/ml activin A, BMP, preferably BMP4, for example 10 ng/ml BMP4; FGF, preferably bFGF (FGF2), for example 5 ng/ml bFGF; and GSK3 inhibitor, preferably CHIR-99021, for example 10 μM CHIR-99021.

A chemically defined medium (CDM) is a nutritive solution for culturing cells which contains only specified components, preferably components of known chemical structure. A CDM is devoid of undefined components or constituents which include undefined components, such as feeder cells, stromal cells, serum, and complex extracellular matrices, such as matrigel™ For example, a CDM does not contain stromal cells, such as OP9 cells, expressing Notch ligands, such as DLL1 or DLL4.

The chemically defined nutrient medium may comprise a chemically defined basal medium. Suitable chemically defined basal media include Iscove's Modified Dulbecco's Medium (IMDM), Ham's F12, Advanced Dulbecco's modified eagle medium (DMEM) (Price et al Focus (2003), 25 3-6), Williams E (Williams, G. M. et al Exp. Cell Research, 89, 139-142 (1974)), RPMI-1640 (Moore, G. E. and Woods L. K., (1976) Tissue Culture Association Manual. 3, 503-508) and StemPro™-34 PLUS (ThermoFisher Scientific).

The basal medium may be supplemented by serum-free culture medium supplements and/or additional components in the medium. Suitable supplements and additional components are described above and may include L-glutamine or substitutes, such as GlutaMAX-1™, ascorbic acid, monothiolglycerol (MTG), antibiotics such as penicillin and streptomycin, human serum albumin, for example recombinant human serum albumin, such as Cellastim™ (Merck/Sigma) and Recombumin™ (albumedix.com), insulin, transferrin and 2-mercaptoethanol. A basal medium may be supplemented with a serum substitute, such as Knockout Serum Replacement (KOSR; Invitrogen).

The iPSCs may be cultured in the first mesoderm induction medium for 1 to 12 hours, for example any of 1, 2, 3, 4, 5, 6, 7, 8, 9, 10, 11 or 12 hours, preferably about 4 hours; then cultured in the second mesoderm induction medium for 30 to 54 hours, for example any of 35, 36, 37, 38, 39, 40, 41, 42, 43, 44, 45, 46, 47, 48, 49 or 50 hours, preferably about 44 hours; and then cultured in the third mesoderm induction medium for 36 to 60 hours,, for example any of 37, 38, 39, 40, 41, 42, 43, 44, 45, 46, 47, 48, 49, 50, 51, 52 or 53 hours, preferably about 48 hours to produce a population of mesodermal cells.

Mesoderm cells are partially differentiated progenitor cells that are committed to mesodermal lineages and are capable of differentiation under appropriate conditions into all cell types in the mesenchyme (fibroblast), muscle, bone, adipose, vascular and haematopoietic systems. Mesoderm cells may express one or more mesodermal markers. For example, the mesoderm cells may express any one, two, three, four, five, six or all seven of Brachyury, Goosecoid, MixI1, KDR, FoxA2, GATA6 and PDGFαR.

In a second stage, mesoderm cells may be differentiated into haemogenic endothelial cells (HECs) by culturing the population of mesoderm cells under suitable conditions to promote haemogenic endothelial (HE) differentiation. For example, the iPSCs cells may be cultured in an HE induction medium.

A suitable HE induction medium may (i) stimulate cKIT receptor (CD117) and/or cKIT receptor (CD117) mediated signaling pathways and (ii) stimulate VEGFR and/or VEGFR mediated signaling pathways. For example, the HE induction medium may comprise SCF and VEGF.

Vascular endothelial growth factor (VEGF) is a protein factor of the PDGF family which binds to VEGFR tyrosine kinase receptors and stimulates vasculogenesis and angiogenesis. Suitable VEGFs include any member of the VEGF family, for example any one of VEGF-A to VEGF-D and PIGF. Preferably, the VEGF is VEGF-A (also known as VEGF, NCBI Gene ID: 7422, nucleic acid sequence NM_001025366.2, amino acid sequence NP_001020537.2). VEGF is readily available from commercial sources (e.g. R&D Systems, USA). Conveniently, the concentration of VEGF in an HE induction medium described herein may be from 1 to 100 ng/ml, for example any of about 2, 3, 4, 5, 6, 7, 8, 9, 10, 12, 15, 16, 17, 18, 19, 20, 25, 30, 35, 40, 45 or 50, 55, 60, 65, 70, 75, 80, 85, 90 or 95 ng/ml, preferably about 15 ng/ml.

In some examples of HE induction media, VEGF may be replaced by a VEGF activator or agonist that stimulates VEGFR mediated signaling pathways. Suitable VEGF activators are known in the art and include proteins, such as gremlin (Mitola et al (2010) Blood 116 (18) 3677-3680) nucleic acids, such as shRNA (e.g. Turunen et al Circ Res. 2009 Sep. 11; 105(6):604-9), CRISPR-based plasmids (e.g. VEGF CRISPR activation plasmid; Santa Cruz Biotech, USA), antibodies and small molecules.

Stem cell factor (SCF) is a cytokine that binds to the KIT receptor (KIT proto-oncogene, receptor tyrosine kinase) (CD117; SCFR) and is involved in haematopoiesis. SCF (also called KITLG, NCBI GeneID: 4254) may have the reference nucleic acid sequence NM_000899.5 or NM_03994.5 and the reference amino acid sequence NP_000890.1 or NP_003985.5. SCF is readily available from commercial sources (e.g. R&D Systems, USA). Conveniently, the concentration of SCF in an HE induction medium described herein may be from 1 to 1000 ng/ml, for example any of about 10, 20, 30, 40, 50, 60, 70, 80, 90, 100, 110, 120, 130, 140, 150, 200, 250, 300, 350, 400, 450, 500, 600, 700, 800, 900 ng/ml, preferably about 100 ng/ml.

In preferred embodiments, the HE induction medium is a chemically defined medium. For example, the HE induction medium may consist of a chemically defined nutrient medium supplemented with effective amounts of VEGF, for example 15 ng/ml VEGF; and SCF, for example 100 ng/ml SCF.

Suitable chemically defined nutrient media are described above and include StemPro™-34 (ThermoFisher Scientific).

The mesoderm cells may be cultured in the HE induction medium for 2 to 6 days or 3 to 5 days, preferably about 4 days, to produce a population of HE cells.

Haemogenic endothelial cells (HECs) are partially differentiated endothelial progenitor cells that have hematopoietic potential and are capable of differentiation under appropriate conditions into haematopoietic lineages. HE cells may express CD34. In some embodiments, HECs may not express CD73 or CXCR4 (CD184). For example, the HE cells may have the phenotype CD34+ CD73− or CD34+ CD73− -CXCR4−.

In a third stage, haemogenic endothelial (HE) cells may be differentiated into haematopoietic progenitor cells (HPCs) by culturing the population of HE cells under suitable conditions to promote haematopoietic differentiation. For example, the HE cells may be cultured in a haematopoietic induction medium.

A suitable haematopoietic induction medium may stimulate the following (i) cKIT receptor (CD117) and/or cKIT receptor (CD117) mediated signaling pathways, (ii) VEGFR and/or VEGFR mediated signaling pathways, (iii) MPL (CD110) and/or MPL (CD110) mediated signaling pathways (iv) FLT3 and/or FLT3 mediated signaling pathways (v) IGF1R and/or IGF1R mediated signaling pathways (vi) SMAD1, 5 and 9 and/or SMAD1, 5 and 9 mediated signaling pathways (vii) Hedgehog and/or Hedgehog signaling pathways (viii) EpoR and/or EpoR mediated signaling pathway and (ix) AGTR2 and/or AGTR2 mediated signaling pathways. A suitable haematopoietic induction medium may also inhibit the AGTR1 (angiotensin II type 1 receptor (AT₁)) and/or AGTR1 (angiotensin II type 1 receptor (AT₁)) mediated signaling pathway. A suitable haematopoietic induction medium may also have interleukin (IL) activity and FGF activity.

For example, a haematopoietic induction medium may comprise the differentiation factors: VEGF, SCF, Thrombopoietin (TPO), Flt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist. An example of a suitable haematopoietic induction medium is the Stage 3 medium shown in Table 1 below.

Thrombopoietin (TPO) is a glycoprotein hormone that regulates platelet production. TPO (also called THPO, NCBI Gene ID: 7066) may have the reference nucleic acid sequence NM_000460.4 and the reference amino acid sequence NP_000451.1. TPO is readily available from commercial sources (e.g. R&D Systems, USA; Miltenyi Biotec Gmbh, DE). Conveniently, the concentration of TPO in a haematopoietic induction medium described herein may be from 3 to 300 ng/ml, for example any of 3, 4, 5, 6, 7, 8, 9, 10, 11, 12, 13, 14, 15, 20, 25, 27, 30, 32, 35, 40, 45, 50, 60, 70, 80, 90 or 100, 110, 120, 130, 140, 150, 160, 170, 180, 190, 200, 225, 250, 275 or 300 ng/ml, preferably about 30 ng/ml.

Flt3 ligand (Fms-related tyrosine kinase 3 ligand or FLT3L) is a cytokine with haematopoietic activity which binds to the FLT3 receptor and stimulates the proliferation and differentiation of progenitor cells. Flt3 ligand (also called FLT3LG, NCBI GeneID: 2323) may have the reference nucleic acid sequence NM_001204502.2 and the reference amino acid sequence NP_001191431.1. Flt3 is readily available from commercial sources (e.g. R&D Systems, USA; Miltenyi Biotec Gmbh, DE). Conveniently, the concentration of Flt3 ligand in a haematopoietic induction medium described herein may be from 0.25 to 250 ng/ml, for example any of about 0.25, 0.5, 1, 2, 3, 4, 5, 6, 7, 8, 9, 10, 11, 12, 13, 14, 15, 20, 25, 30, 35, 40, 45, 50, 60, 70, 80, 90, 100, 125, 150, 175, 200, 225 or 250 ng/ml, preferably about 25 ng/ml.

Interleukins (ILs) are cytokines that play major roles in immune development and function. ILs in a haematopoietic induction medium may include IL-3, IL-6, IL-7, and IL-11.

IL-3 (also called IL3 or MCGF, NCBI GeneID: 3562) may have the reference nucleic acid sequence NM_000588.4 and the reference amino acid sequence NP_000579.2. IL-3 is readily available from commercial sources (e.g. R&D Systems, USA; Miltenyi Biotec Gmbh, DE). Conveniently, the concentration of IL-3 in a haematopoietic induction medium described herein may be from 0.25 to 250 ng/ml, for example any of about 0.25, 0.5, 1, 2, 3, 4, 5, 6, 7, 8, 9, 10, 11, 12, 13, 14, 15, 20, 25, 30, 35, 40, 45, 50, 60, 70, 80, 90, 100, 125, 150, 175, 200, 225 or 250 ng/ml, preferably about 25 ng/ml.

IL-6 (also called IL6 or HGF, NCBI GeneID: 3569) may have the reference nucleic acid sequence NM_000600.5 and the reference amino acid sequence NP_000591.5. IL-6 is readily available from commercial sources (e.g. R&D Systems, USA; Miltenyi Biotec Gmbh, DE). Conveniently, the concentration of IL-6 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml preferably about 10 ng/ml.

IL-7 (also called IL7, NCBI GeneID: 3574) may have the reference nucleic acid sequence NM_000880.4 and the reference amino acid sequence NP_000871.1. IL-7 is readily available from commercial sources (e.g. R&D Systems, USA; Miltenyi Biotec Gmbh, DE). Conveniently, the concentration of IL-7 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-11 (also called AGIF, NCBI GeneID: 3589) may have the reference nucleic acid sequence NM_000641.4 and the reference amino acid sequence NP_000632.1. IL-11 is readily available from commercial sources (e.g. R&D Systems, USA; Miltenyi Biotec Gmbh, DE). Conveniently, the concentration of IL-11 ligand in a haematopoietic induction medium described herein may be from 0.5 to 100 ng/ml, for example any of about 0.5, 0.75, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 5 ng/ml.

Insulin-like growth factor 1 (IGF-1) is a hormone that binds to the tyrosine kinases IGF-1 receptor (IGF1R) and insulin receptor and activates the multiple signaling pathways. IGF-1 (also called IGF or MGF, NCBI GeneID: 3479) may have the reference nucleic acid sequence NM_000618.5 and the reference amino acid sequence NP_000609.1. IGF-1 is readily available from commercial sources (e.g. R&D Systems, USA). Conveniently, the concentration of IGF-1 in a haematopoietic induction medium described herein may be from 0.25 to 250 ng/ml, for example any of about 0.25, 0.5, 1, 2, 3, 4, 5, 6, 7, 8, 9, 10, 11, 12, 13, 14, 15, 17, 20, 22, 25, 27, 30, 35, 40, 45, 50, 60, 70, 80, 90, 100, 125, 150, 175, 200, 225 or 250 ng/ml, preferably about 25 ng/ml.

Sonic hedgehog (SHH) is a ligand of the hedgehog signaling pathway that regulates vertebrate organogenesis. SHH (also called TPT or HHG1, NCBI GeneID: 6469) may have the reference nucleic acid sequence NM_000193.4 and the reference amino acid sequence NP_000184.1. SHH is readily available from commercial sources (e.g. R&D Systems, USA; Miltenyi Biotec Gmbh, DE). Conveniently, the concentration of SHH in a haematopoietic induction medium described herein may be from 0.25 to 250ng/ml, for example any of about 0.25, 0.5, 1, 2, 3, 4, 5, 6, 7, 8, 9, 10, 11, 12, 13, 14, 15, 17, 20, 22, 25, 27, 30, 35, 40, 45, 50, 60, 70, 80, 90, 100, 125, 150, 175, 200, 225 or 250 ng/ml, preferably about 25 ng/ml.

Erythropoietin (EPO) is a glycoprotein cytokine that binds to the erythropoietin receptor (EpoR) and stimulates erythropoiesis. EPO (also called DBAL, NCBI GeneID: 2056) may have the reference nucleic acid sequence NM_000799.4 and the reference amino acid sequence NP_000790.2. EPO is readily available from commercial sources (e.g. R&D Systems, USA; PreproTech, USA). Conveniently, the concentration of EPO in haematopoietic induction medium described herein may be from 0.02 to 20 U/ml, for example any of about 0.025, 0.05, 0.075, 0.1, 0.5, 0.75, 1.0, 1.5, 2.0, 2.5, 3, 4, 5, 6, 7, 8, 9, 10 13, 15, 17, or 19 U/ml, preferably about 2 U/ml.

Angiotensin II is a heptapeptide hormone that is formed by the action of angiotensin converting enzyme (ACE) on angiotensin I. Angiotensin II stimulates vasoconstriction. Angiotensin I and II are formed by the cleavage of angiotensinogen (also called AGT, NCBI GeneID: 183), which may have the reference nucleic acid sequence NM_000029.4 and the reference amino acid sequence NP_000020.1. Angiotensin II is readily available from commercial sources (e.g. R&D Systems, USA; Tocris, USA). Conveniently, the concentration of angiotensin II in a haematopoietic induction medium described herein may be from 0.05 to 50 ng/ml, for example any of about 0.05, 0.075, 0.1, 0.5, 0.75, 1.0, 1.5, 2.0, 2.5, 3, 4, 5, 6, 7, 8, 9, 10, 15, 20, 30, 40 or 50 ng/ml preferably about 5 ng/ml.

Angiotensin II type 1 receptor (AT1) antagonists (ARBs) are compounds that selectively block the activation of AT₁ receptor (AGTR1; Gene ID 185). Suitable AT₁ antagonists include losartan (2-Butyl-4-chloro-1-{[2′-(1H-tetrazol-5-yl)-4-biphenylyl]methyl}-1H-imidazol-5-yl)methanol), valsartan ((2S)-3-Methyl-2-(pentanoyl{[2′-(1H-tetrazol-5-yl) biphenyl-4-yl]methyl}amino)butanoic acid), and telmisartan (4′[(1,4′-Dimethyl-2′-propyl [2,6′-bi-1H-benzimidazol]-1′-yl) methyl][1, 1′-biphenyl]-2-carboxylic acid. In some preferred embodiments, the AT₁ antagonist is losartan. Suitable AT1 antagonists may be obtained from commercial suppliers (e.g. Tocris, USA; Cayman Chemical Co. MI USA). Conveniently, the concentration of angiotensin II type 1 receptor (AT₁) antagonist in a haematopoietic induction medium described herein may be from 1 to 1000 μM, for example any of about 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 600, 700, 800, 900 ng/ml, preferably about 100 μM.

In preferred embodiments, the haematopoietic induction medium is a chemically defined medium. For example, the haematopoietic induction medium may consist of a chemically defined nutrient medium supplemented with effective amounts of VEGF, for example 15 ng/ml; SCF, for example 100 ng/ml; thrombopoietin (TPO), for example 30 ng/ml; Flt3 ligand (FLT3L), for example 25 ng./ml; IL-3, for example 25 ng/ml; IL-6, for example 10 ng/ml; IL-7, for example 10 ng/ml; IL-11, for example 5 ng/ml; IGF-1, for example 25 ng/ml; BMP, for example BMP4 at 10 ng/ml; FGF, for example bFGF at 5 ng/ml; Sonic hedgehog (SHH), for example 25 ng/ml; erythropoietin (EPO), for example 2 U/ml; angiotensin II, for example 10 μg/ml, and an angiotensin II type 1 receptor (AT₁) antagonist, for example losartan, at 100 μM. A suitable haematopoietic induction medium be devoid of other differentiation factors. For example, a haematopoietic induction medium may consist of a chemically defined nutrient medium supplemented with one or more differentiation factors, wherein the one or more differentiation factors consist of VEGF, SCF, Thrombopoietin (TPO), Flt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT1) antagonist (i.e. the medium does not contain any differentiation factors other than VEGF, SCF, Thrombopoietin (TPO), Flt3 ligand (FIt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist).

Suitable chemically defined nutrient media are described above and include StemPro™-34PLUS (ThermoFisher Scientific) or a basal medium such as IMDM supplemented with albumin, insulin, selenium transferrin, and lipids as described below.

The HE cells may be cultured in the haematopoietic induction medium for 8-21 days, for example any of about 9, 10, 11, 12, 13, 14, 15, 16, 17, 18, 19 or 20 days, preferably about 16 days, to produce the population of HPCs.

HPCs (also called hematopoietic stem cells) are multipotent stem cells that are committed to a hematopoietic lineage and are capable of further hematopoietic differentiation into all blood cell types including myeloid and lymphoid lineages, including monocytes, B cells and T cells. HPCs may express CD34. HPCs may co-express CD133, CD45 and FLK1 (also known as KDR or VEGFR2) and may be negative for expression of CD38 and other lineage specific markers. For example, HPCs may display the phenotype CD34+ CD133+ CD45+ FLK1+ CD38−.

Following the generation of HPCs from HE cells, a population of HPCs expressing one or more cell surface markers, such as CD34, may be purified, for example by magnetic activated cell sorting (MACS), before being subjected to further differentiation. For example, a population of CD34+ HPCs may be purified. The CD34+ HPCs may be purified after 8 days, for example 8, 9 or 10 days, culture in the HE induction medium. The CD34+ HPCs may be purified after 16 days of differentiation, for example on day 16, 17 or 18 of the differentiation method.

In a fourth stage, haematopoietic progenitor cells (HPCs) may be differentiated into progenitor T cells by culturing the population of HPCs under suitable conditions to promote lymphoid differentiation. For example, the haematopoietic progenitor cells may be cultured in a lymphoid expansion medium.

A lymphoid expansion medium is a cell culture medium that promotes the lymphoid differentiation of HPCs into progenitor T cells.

A suitable lymphoid expansion medium may (i) stimulate cKIT receptor (CD117; KIT receptor tyrosine kinase) and/or cKIT receptor (CD117; KIT receptor tyrosine kinase) mediated signaling pathways, (ii) stimulate MPL (CD110) and/or MPL (CD110) mediated signaling pathways (iii) FLT3 and/or FLT3 mediated signaling pathways and (iv) have interleukin (IL) activity. For example, a lymphoid expansion medium may comprise the differentiation factors SCF, FLT3L, TPO and IL7.

In preferred embodiments, the lymphoid expansion medium is a chemically defined medium. For example, the lymphoid expansion medium may consist of a chemically defined nutrient medium supplemented with effective amounts of the above differentiation factors. Suitable lymphoid expansion media are well-known in the art and include Stemspan™ SFEM II (Cat #9605; StemCell Technologies Inc, CA).with Stemspan™ lymphoid expansion supplement (Cat #9915; StemCell Technologies Inc, CA).

The HPCs may be cultured on a surface during differentiation into progenitor T cells. For example, the HPCs may be cultured on a surface of a culture vessel, bead or other biomaterial or polymer.

Preferably, the surface may be coated with a factor that stimulates Notch signaling, for example a Notch ligand, such as Delta-like 1 (DLL1) or Delta-like 4 (DLL4). Suitable Notch ligands are well-known in the art and available from commercial suppliers.

The surface may also be coated with an extracellular matrix protein, such as fibronectin, vitronectin, laminin or collagen and/or one or more cell surface adhesion proteins, such as VCAM1. In some embodiments, the surface for HPC culture may have a coating that comprises a factor that stimulates Notch signaling, for example a Notch ligand, such as DLL4, without the extracellular matrix protein or cell surface adhesion protein.

In some embodiments, the surface for HPC culture may have a coating that comprises a factor that stimulates Notch signaling, for example a Notch ligand, such as DLL4, an extracellular matrix protein, such as vitronectin, and a cell surface adhesion protein, such as VCAM1. The surface may be coated with an extracellular matrix protein, factor that stimulates Notch signaling and cell surface adhesion protein by contacting the surface with a coating solution. For example, the coating solution may be incubated on the surface under suitable conditions to coat the surface. Conditions may, for example, include about 2 hours at room temperature. Coating solutions comprising an extracellular matrix protein and a factor that stimulates Notch signaling are available from commercial suppliers (StemSpan™ Lymphoid Differentiation Coating Material; Cat #9925; Stem Cell Technologies Inc, CA).

The HPCs may be cultured in the lymphoid expansion medium on the substrate or surface for a time sufficient for the HPCs to differentiate into progenitor T cells. For example, the HPCs may be cultured for 2-6 weeks, 2 to 5 weeks or 2-4 weeks, preferably 3 weeks.

Progenitor T cells are multi-potent lymphopoietic progenitor cells that are capable of giving rise to αβ T cells, γδ T cells, tissue resident T cells and NK T cells. Progenitor T cells may commit to the αβ T cell lineage after pre-TCR selection in the thymus. Progenitor T cells may be capable of in vivo thymus colonization and may be capable of committing to the T cell lineage after pre-TCR selection in the thymus. Progenitor T cells may also be capable of maturation into cytokine-producing CD3⁺ T-cells.

Progenitor T cells may express CD5 and CD7 i.e. the progenitor T cells may have a CD5+CD7+ phenotype. Progenitor T cells may also co-express CD44, CD25 and CD2. For example, progenitor T cells may have a CD5+, CD7+CD44+, CD25+CD2+ phenotype. In some embodiments, progenitor T cells may also co-express CD45. Progenitor T cells may lack expression of CD3, CD4 and CD8, for example on the cell surface.

In a fifth stage, progenitor T cells may be matured into T cells by culturing the population of progenitor T cells under suitable conditions to promote T cell maturation. For example, the progenitor T cells may be cultured in a T cell maturation medium.

A T cell maturation medium is a cell culture medium that promotes the maturation of progenitor T cells into mature T cells. A suitable T cell maturation medium may (i) stimulate cKIT receptor (CD117; KIT receptor tyrosine kinase) and/or cKIT receptor (CD117; KIT receptor tyrosine kinase) mediated signaling pathways (ii) FLT3 and/or FLT3 mediated signaling pathways and (iii) have interleukin (IL) activity. For example, a T cell maturation medium may comprise the differentiation factors SCF, FLT3L, and IL7.

In preferred embodiments, the T cell maturation medium is a chemically defined medium. For example, the T cell maturation medium may consist of a chemically defined nutrient medium supplemented with effective amounts of the above differentiation factors. Suitable T cell maturation media are well-known in the art and include Stemspan™ SFEM II (Cat #9605; StemCell Technologies Inc, CA) with Stemspan™ T cell maturation supplement (Cat #9930; StemCell Technologies Inc, CA) and other media suitable for expansion of PBMCs and CD3+ cells, such as ExCellerate Human T cell expansion medium (R& D Systems, USA). Other suitable T cell maturation media may include a basal medium such as IMDM, supplemented with ITS, albumin and lipids, as described elsewhere herein and further supplemented with effective amounts of the above differentiation factors.

The progenitor T cells may be cultured on a surface. For example, the progenitor T cells may be cultured on a surface of a culture vessel, bead or other biomaterial or polymer.

Preferably, the surface may be coated with a factor that stimulates Notch signaling, for example a Notch ligand, such as Delta-like 1 (DLL1) or Delta-like 4 (DLL4). Suitable Notch ligands are well-known in the art and available from commercial suppliers. The surface may also be coated with an extracellular matrix protein, such as fibronectin, vitronectin, laminin or collagen and/or one or more cell surface adhesion proteins, such as VCAM1. Suitable coatings are well-known in the art and described elsewhere herein.

The progenitor T cells may be cultured in the T cell maturation medium on the substrate or surface for a time sufficient for the progenitor T cells to mature into T cells. For example, the progenitor T cells may be cultured for 1-4 weeks, preferably 2 or 3 weeks.

In some embodiments, the T cells produced by maturation of progenitor T cells may be double positive CD4+CD8+ T cells.

Progenitor T cells may be matured into T cells by the methods described above. T cells (also called T lymphocytes) are white blood cells that play a central role in cell-mediated immunity. T cells can be distinguished from other lymphocytes by the presence of a T cell receptor (TCR) on the cell surface. There are several types of T cells, each type having a distinct function.

T helper cells (TH cells) are known as CD4⁺ T cells because they express the CD4 surface glycoprotein. CD4⁺ T cells play an important role in the adaptive immune system and help the activity of other immune cells by releasing T cell cytokines and helping to suppress or regulate immune responses. They are essential for the activation and growth of CD4+ CD8+ T cells.

Cytotoxic T cells (T_{C} cells, CTLs, killer T cells, CD4+ CD8+T cells) are known as CD8⁺ T cells because they express the CD8 surface glycoprotein. CD8⁺ T cells act to destroy virus-infected cells and tumour cells. Most CD8⁺ T cells express TCRs that can recognise a specific antigen displayed on the surface of infected or damaged cells by a class I MHC molecule. Specific binding of the TCR and CD8 glycoprotein to the antigen and MHC molecule leads to T cell-mediated destruction of the infected or damaged cells.

T cells produced as described herein may be double positive CD4+CD8+ T cells or single positive CD4+ or CD8+ T cells.

T cells produced as described herein may be mature CD3+ T cells. For example, the cells may have a TCR+ CD3+ CD8+ CD4+ phenotype. In some embodiments, T cells may also express CD45 and CD28.

T cells produced as described herein may be γδ T cells, αβ T cells or NKT cells.

In some preferred embodiments, the T cells produced as described herein are αβ T cells.

Following maturation of progenitor T cells (stage 5), the population of T cells may be predominantly double positive CD4+CD8+ T cells.

In a sixth stage, the population of TCR T cells may be activated and/or expanded to produce or increase the proportion of single positive CD4+ T cells, or more preferably single positive CD8+ T cells. Suitable methods for activating and expanding T cells are well-known in the art. For example, T cells may be exposed to a T cell receptor (TCR) agonist under appropriate culture conditions. Suitable TCR agonists include ligands, such as peptides displayed on a class I or II MHC molecule (MHC-peptide complexes) on the surface of a bead or an antigen presenting cell, such as a dendritic cell, and soluble factors, such as anti-TCR antibodies for example antibody CD28 antibodies, and multimeric MHC-peptide complexes, such as MHC-peptide tetramers, pentamers or dextramers.

Activation refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA). In some embodiments, T cells may be activated by exposure to anti-αCD3 antibodies and IL2, IL7 or IL15. More preferably, T cells are activated by exposure to anti-αCD3 antibodies and anti-αCD28 antibodies. The activation may occur in the presence or absence of CD14⁺ monocytes. The T cells may be activated with anti-CD3 and anti-CD28 antibody coated beads. For example, PBMCs or T cell subsets including CD4⁺ and/or CD8⁺ cells may be activated, without feeder cells (antigen presenting cells) or antigen, using antibody coated beads, for example magnetic beads coated with anti-CD3 and anti-CD28 antibodies, such as Dynabeads® Human T-Activator CD3/CD28 (ThermoFisher Scientific). In other embodiments, soluble tetrameric antibody complexes that bind CD3, CD28 and CD2 cell surface ligands, such as ImmunoCult™ Human CD3/CD28/CD2 T Cell Activator or Human CD3/CD28 T Cell Activator, may be used to activate the T cells. In other embodiments, T cells may be activated with an MHC-peptide complex, preferably a multimeric MHC-peptide complex, optionally in combination with an anti-CD28 antibody.

In some embodiments, double positive CD4+CD8+ T cells may be cultured in a T cell maturation medium as described herein supplemented with IL-15. The medium may be further supplemented with a T cell receptor (TCR) agonist, for example one or more anti-TCR antibodies, such as anti-αCD3 antibodies, and anti-αCD28 antibodies, as described above in order to activate and expand the population and produce single positive T cells.

The T cells may be cultured using any convenient technique to produce the expanded population. Suitable culture systems include stirred tank fermenters, airlift fermenters, roller bottles, culture bags or dishes, and other bioreactors, in particular hollow fibre bioreactors.

The use of such systems is well-known in the art

T cells produced as described herein may express a heterologous T cell receptor (TCR) that binds a target antigen. For example, the heterologous TCR may bind specifically to cancer cells that express a tumor antigen or peptide fragment thereof. In some embodiments, the T-cells may be RAG inactivated (i.e. the RAG1 and/or RAG2 genes in the cells are knocked out by a deletion or other mutation), such that they do not express endogenous TCR. The T cells may be useful for example in immunotherapy, as described below.

Following production, the population of T cells, for example (double positive) DP CD4+CD8+ cells, (single positive) SP CD4+ cells or SP CD8+ cells, may be isolated and/or purified. Any convenient technique may be used, including fluorescence-activated cell sorting (FACS) or magnetic-activated cell sorting using antibody coated magnetic particles (MACS).

The population of T cells, for example DP CD4+CD8+ cells, SP CD4+ cells or SP CD8+ cells, may be expanded and/or concentrated. Optionally, the population of T cells produced as described herein may be stored, for example by cryopreservation, before use.

### Treatment

The present invention further provides a pharmaceutical composition comprising the modified iPSC or haemogenic lineage cell, preferably T cell, according to the invention and a pharmaceutically acceptable carrier.

The present invention further provides a modified iPSC or haemogenic lineage cell preferably a T cell, or pharmaceutical composition according to the invention, for use in therapy and/or medicine.

The invention also provides any one or more of the following medical uses or methods of treatment comprising:
(a) a modified iPSC or haemogenic lineage cell, preferably T cell, or pharmaceutical composition according to the invention, for use in treatment, prevention or delaying the progression of cancer and/or tumour in an individual or subject optionally wherein the treatment is cancer immunotherapy therapy and/or adoptive T cell therapy, optionally allogenic adoptive T cell therapy,
(b) use of a modified iPSC or haemogenic lineage cell, preferably T cell, or pharmaceutical composition according to the invention, in the manufacture of a medicament for the treatment of cancer and/or tumour in an individual or subject, optionally wherein the treatment is cancer immunotherapy therapy and/or adoptive T cell therapy, optionally allogenic adoptive T cell therapy,
(c) a method of treating, preventing or delaying the progression of cancer and/or tumour in an individual or subject comprising administering to the individual the modified iPSC or haemogenic lineage cell, preferably T cell, or pharmaceutical composition according to the invention, optionally wherein the treatment is cancer immunotherapy therapy and/or adoptive T cell therapy, optionally allogenic adoptive T cell therapy.

According to the present invention, the cancer and/or tumour, which may be a solid tumour, may be selected from; lung cancer, non-small cell lung cancer (NSCLC), metastatic or advanced NSCLC, squamous NSCLC, adenocarcinoma NSCLC, adenosquamous NSCLC, large cell NSCLC, ovarian cancer, gastric cancer, urothelial cancer, esophageal cancer, esophagogastric junction cancer (EGJ), melanoma, bladder cancer, head and neck cancer, head and neck squamous cell carcinoma (HNSCC), cancer of the oral cavity, cancer of the oropharynx, cancer of the hypopharynx, cancer of the throat, cancer of the larynx, cancer of the tonsil, cancer of the tongue, cancer of the soft palate, cancer of the pharynx, synovial sarcoma, myxoid round cell liposarcoma (MRCLS): optionally wherein the cancer or tumour express a MAGE or AFP protein, peptide, antigen or peptide antigen thereof as described herein, optionally MAGE-A4 protein, peptide, antigen or peptide antigen thereof as described herein.

According to the present invention, the cancer may be selected from any one or more of; breast cancer, metastatic breast cancer, liver cancer, renal cell carcinoma, synovial sarcoma, urothelial cancer or tumour, pancreatic cancer, colorectal cancer, metastatic stomach cancer, metastatic gastric cancer, metastatic liver cancer, metastatic ovarian cancer, metastatic pancreatic cancer, metastatic colorectal cancer, metastatic lung cancer, colorectal carcinoma or adenocarcinoma, lung carcinoma or adenocarcinoma, pancreatic carcinoma or adenocarcinoma, mucinous adenoma, ductal carcinoma of the pancreas, and hematological malignancy: optionally wherein the cancer or tumour express a MAGE or AFP protein, peptide, antigen or peptide antigen thereof as described herein, optionally MAGE-A4 protein, peptide, antigen or peptide antigen thereof as described herein.

The cancer and/or tumour may be liver cancer, for example liver cancer selected from any of; cholangiocarcinoma, liver angiosarcoma, hepatoblastoma, hepatocellular carcinoma (HCC), optionally wherein the cancer is not amenable to transplant or resection, preferably the cancer is hepatocellular carcinoma (HCC); additionally the liver cancer may be coincident with any one or more of; diabetes, obesity, hepatitis B, hepatitis C, cirrhosis: optionally wherein the cancer or tumour express a MAGE or AFP protein, peptide, antigen or peptide antigen thereof as described herein, optionally MAGE-A4 protein, peptide, antigen or peptide antigen thereof as described herein.

According to the present invention, the cancer can be relapsed cancer or refractory cancer or recurrent cancer or locally recurrent cancer or metastatic cancer, non-resectable cancer or locally confined, cancer with no surgical or radiotherapy option or inoperable cancer, or any combination thereof. The cancer and/or tumour may be a solid tumour.

The invention also provides a modified iPSC or haemogenic lineage cell, preferably T cell, or pharmaceutical composition according to the invention, for use in a method of,
(a) up-regulation of T cell activation,
(b) up-regulation of cytokine production, for example any one or more of IFN gamma, IL-2 or Granzyme B,
(c) induction of cell cytotoxic activity in the presence of the antigen or antigen peptide, or
(d) inducing the killing of tumour cells presenting the antigen or antigen peptide; optionally in a subject with cancer and/or tumour, optionally wherein the cancer and/or tumour express a cancer and/or tumour antigen or peptide antigen thereof as described herein, preferably a MAGE or AFP protein, peptide, antigen or peptide antigen thereof as described herein.

### Combinations

The present invention further provides a modified iPSC or haemogenic lineage cell, preferably T cell, or pharmaceutical composition according to the invention, for use according to the forgoing medical uses or methods of treatment, wherein the modified iPSC or haemogenic lineage cell, preferably T cell, or pharmaceutical composition is for use, or is used, in combination with one or more further therapeutic agent optionally administered or for administration separately, sequentially or simultaneously.

Accordingly, the one or more further therapeutic agent can comprise, chemotherapy agent, hormonal therapy agent, a targeted drug, or immunotherapy. Accordingly, the chemotherapy may comprise any one or more of, gemcitabine, docetaxel, pemetrexed, topotecan, irinotecan, etoposide, vinorelbine Lipoplatin, Cisplatin, Carboplatin, Oxaliplatin, Nedaplatin, Triplatin tetranitrate, Phenanthriplatin, Satraplatin, Picoplatin, methotrexate, capecitabine, taxane, anthracycline, paclitaxel, docetaxel, paclitaxel protein bound particles, doxorubicine, epirubicine, 5-fluorouracil, cyclophosphamide, vincristine, etoposide. Additionally, or alternatively the chemotherapy may comprise one or more chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide. Additionally or alternatively the one or more further therapeutic agent can comprise any one or more of Sorafenib, a PD1 or PD-L1 antagonist or inhibitor, Regorafenib, Cabozantinib, Sunitinib Brivanib, Everolimus, Tivantinib, Linifanibfrom Sorafenib, a PD1 or PD-L1 antagonist or inhibitor, Regorafenib, Cabozantinib, Sunitinib, Brivanib, Everolimus, Tivantinib, Linifanib, or a tyrosine kinase inhibitor or epidermal growth factor receptor (EGFR) inhibitor, such as crizotinib, erlotinib, gefitinib and afatinib, an immune checkpoint inhibitor, such as pembrolizumab or nivolumab, or monoclonal antibody against nuclear factor kappa-B ligand, such as denosumab, or an Epidermal Growth Factor Receptor Antagonist antibody, optionally Cetuximab.

As used herein the term “modified iPSC or haemogenic lineage cell” may also include modified induced pluripotent stem cells iPSCs or haemogenic lineage cells or modified population of induced pluripotent stem cells iPSCs or population haemogenic lineage cells, (wherein the term modified also applies to both IPSC(s) and haemogenic lineage cell(s)).

### Kits

The present invention further provides a kit comprising,
(a) the modified iPSC or haemogenic lineage cell, preferably T cell, or pharmaceutical composition according to the invention, and a package insert comprising instructions for use thereof for treating, preventing or delaying the progression of cancer and/or tumour in an individual or subject, or
(b) the modified iPSC or haemogenic lineage cell, preferably T cell, or pharmaceutical composition according to the invention, and a package insert comprising instructions for use thereof for treating, preventing or delaying the progression of cancer and/or tumour in an individual or subject, in combination with one or more further therapeutic agent, optionally as herein described, optionally administered or for administration separately, sequentially or simultaneously.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

Any section headings used herein are for organizational purposes only and are not to be construed as limiting the subject matter described.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### EXPERIMENTS

### Introduction

We describe here the generation of iT-cells expressing a defined heterologous recombinant αβTCR. Starting with a GMP compliant hiPSC source, we knocked-in the recombinant heterologous T-cell receptor ADP A2M4 that recognises the MAGE-A4 peptide (GVYDGREHTV) presented by HLA-A*02. We show that ADP A2M4 iT-cells derived from engineered iPSCs specifically express the ADP A2M4 TCR as measured by anti-TCRVα24 and dextramer staining. Edited ADP A2M4 iT-cells up regulate activation markers including CD25 and CD69 when incubated with HLA-A*02 expressing tumour lines that express the cognate antigen and exhibit potent antigen dependent killing of these lines. The following examples represents the development of an allogeneic hiPSC derived platform, with limited genome editing, that permits the production of ADP A2M4 TCR iT-cells of therapeutic value and activity.

ADP A2M4 TCR is stably introduced into the iT-cells by knock-in at the EEF1A1 locus. An rAAV targeting plasmid was generated to enable the knock-in of the ADP A2M4 TCR into EEF1A1 exon 8. The construct was designed to generate a multicistronic fusion gene between EEF1A1 and ADP A2M4 SPEAR. This permits the regulated transcription and expression of EEF1A1 and ADP A2M4 from the endogenous EEF1A1 promoter. A furin cleavage site combined with T2A or P2A skip sequence peptide separates the EEF1A1 and ADP A2M4 TCRα and A2M4 TCRβ coding sequences (FIG. 2). This allows the production of multiple polypeptides from a single mRNA. Cleavage by the furin protease in the endoplasmic reticulum will remove the skip sequence peptides allowing the expression of nascent protein sequences.

### Production of iPSC Cell Lines

The iPSC line (HCP1324_c1A12) is a derivative of GR1.1, which was created via re-programming of CD34+ progenitors isolated from umbilical cord blood. [Baghbaderani B A, Syama A, Sivapatham R, Pei Y, Mukherjee O, Fellner T, Zeng, Rao M S, Detailed Characterization of Human Induced Pluripotent Stem Cells Manufactured for Therapeutic Applications. Stem Cell Rev. 2016 Jun. 10] Genomic stability was assessed via cyto-SNP analysis, karyotyping prior to TCR editing. Pluripotency was determined with IHC (immunohistochemistry), flow cytometry and Pluritest [Muller, F. J., et al., A bioinformatic assay for pluripotency in human cells. Nat Methods, 2011. 8 (4): p. 315-7]. Differentiation of iT-cells from HCP1324_c1A12 iPSC clones was also confirmed.

### iPSC Cell Culture

The iPSC line HCP1324_c1A12 was used for editing experiments. The iPSC line was cultured on Vitronectin with mTeSR™ Plus culture media (STEMCELL Technologies) and adapted for enzymatic dissociation with TrypLE™ Select (ThermoFisher) and passaged every 4-5 days. iPSC cultures were maintained in a humidified 37° C., 5% O₂, 5% CO₂ incubator. The expression of pluripotency markers (POU5F1, NANOG, TRA-160 and SOX2) and absence of the differentiation marker SSEA-1 was routinely monitored by FACS analysis.

### Generation of the AAV Targeting Vector

The pAAV-MCS (Agilent) plasmid was digested with NotI (NEB) to remove the sequences between the left and right inverted terminal repeat (ITR) regions. The vector backbone was purified by agarose gel extraction. The left and right homology arms (LHA, SEQ ID No: 43 and RHA, SEQ ID No: 44), FuTA_A2M4 transgene cassette, SEQ ID No: 45, and P2A_PURO cassette, SEQ ID No: 48, were amplified by PCR. Each primer contained overlapping sequence to allow generation of the targeting vector by Gibson assembly. The left and right homology arm sequences (500 bp) were amplified from genomic DNA (gDNA) isolated from the iPSC line HCP1324_c1A12. The ADP A2M4 TCR transgene contains the coding sequence of the TCRα and TCRβ chains with a furin cleavage site (RAKR) and P2A skip sequence between two TCR chains.

### AAV Production

The packaging plasmids pHelper (Agilent 240071) and AAV-3 Rep-Cap Plasmid (Cell Biolabs VPK-423) used for rAAV production were grown in NEBR 5-alpha Competent E. coli. The AAV targeting plasmid containing the homology arms, P2A-Puro_T2A_A2M4 transgene cassette were grown in NEBR Stable Competent E. coli. All plasmid DNA was purified using Plasmid Plus Giga Kit (Qiagen) or EndoFree Plasmid Giga Kit (Qiagen). The ITR integrity in the AAV transgene plasmid was confirmed with Ahdl and Bgll single restriction enzyme digest.

The 293AAV Cell Line (Cell Biolabs) used to produce rAAV was cultured with 10% (v/v) Heat Inactivated FBS (Life Technologies), DMEM, high glucose, GlutaMAX™ Supplement, pyruvate (Life technologies) Pen/Strep (Life Technologies) and Non Essential Amino Acids (Life Technologies). 293AAV were seeded in a 5-cell stack (Corning) at a density of 216×106/5CS 24 hrs before transfection. 293AAV were transfected with the rAAV transgene plasmid, pHelper and AAV-3 Rep-Cap at a molar ratio of 2:1:1 using Turbofect transfection reagent (Thermofisher). A total 2.6 mg plasmid DNA was used for each transfection. Cells were cultured for 72 hrs before harvesting and virus purification. To harvest cells, EDTA (Thermofisher) was added to a final concentration of 6.25 mM to promote cell detachment from the tissue culture plastic. rAAV virus was purified via iodixanol gradient ultracentrifugation or the AAVpro® Purification Kit (Takara) according to the manufacturer's instructions.

For iodixanol gradient purification, cell pellets were collected by centrifugation (300 g, 5 min), resuspended in 5 ml cell lysis buffer (150 mM NaCl, 5 mM Tris-HCl PH 8.5) and subjected to three rounds of freeze thaw using a dry-ice/ethanol bath and a 37° C. water bath. Following the final thaw, the lysate was treated with Benzonase (200 U/ml) (Sigma) for 1 hr at 37° C. Remaining debris was removed by centrifugation (1000 g, 29 min, 4° C.) before loading onto the iodixanol gradient. The iodixanol gradient (9 ml 15% iodixanol, 6 ml 25% iodixanol, 5 ml 40% iodixanol and 5 ml, 60% iodixanol) was prepared by diluting 60% iodixanol (OptiPrep), (STEMCELL Technologies). The 25% and 40% iodixanol phases were prepared by diluting 60% iodixanol in PBS-MK buffer (1×PBS, 2.76 mM MgCl₂, 2 mM KCl). The 15% iodixanol phase was prepared by diluting 60% iodixanol in PBS-MK buffer (1×PBS 1 M NaCl, 2.76 mM MgCl₂, 2 mM KCl). Phenol red was added to the 25% and 60% phases into order to distinguish these layers. Gradient phases are added step wise into OptiSeal Polypropylene Centrifuge tubes (Beckman Coulter). The cell lysate is loaded on top of the 15% phase and tubes centrifuged in a 70Ti ultracentrifuge rotor at 67,000 rpm for 90 min at 18° C. rAAV is extracted from the 40% phase following centrifugation. rAAV virus preps were concentrated further using centrifugal filter units (MWCO 100 kDa) (Millipore) and the buffer was exchanged for PBS 0.001% (v/v) Pluronic Acid (Sigma). Virus preps were stored at −80° C. Viral titres were confirmed by qPCR using the QuantiTect Probe PCR kit (Qiagen). Primer and probe sequences are designed against the ITR (Aurnhamer et al., 2012. Human Gene Ther Methods). Primer sequences ITR_F GGAACCCCTAGTGATGGAGTT, SEQ ID NO: 54, ITR_R CGGCCTCAGTGAGCGA SEQ ID NO: 55, Probe 56-FAM/CACTCCCTC/ZEN/TCTGCGCGCTCG/3IABKFQ. Viral quantification was determined against a standard curve prepared from pAAVS derived plasmid DNA of known concentration and copy number. The PCR program consisted of one cycle of denaturation at 95° C. for 15 min followed by 40 cycles of denaturation at 95° C. for 15 s and primer annealing/extension at 60° C. for 60 s. qPCR reactions were performed on the QuantStudio 7 and analysed with the QuantStudio™ Real Time PCR software.

### Gene Editing—Knock-In of A2M4 SPEAR Into Exon 8 at the EEF1a Locus

iPSC line, HCP1324_c1A12, was passaged enzymatically using TrypLE Select (ThermoFisher) as a single cell suspension. Following dissociation, iPSCs were counted and seeded in a well of a coated (vitronectin) 6-well plate containing complete mTeSR™ plus. Media was exchanged for complete mTeSR™ plus the following day. iPSCs were cultured for 48 hours prior to AAV transduction.

iPSCs were transduced with AAV3 serotyped virus (500-10000 Vg/cell). Antibiotic selection (puromycin, 30-100 μg/ml) was commenced 48 hrs post transduction and was continued throughout the remaining culture. 3-6 days after viral transduction, iPSCs were seeded into coated 96 well plates for screening and clonal isolation. Clones were expanded in 96 well plates for 10-16 days before PCR screening. In some instances, positive clones were subjected to additional clonal isolation by limiting dilution. All clones were screened by PCR to confirm integration. All PCR products were sequence verified.

### Clone Isolation and Screening

iPSC clones were isolated following selection of the edited iPSC cell pools with puromycin. Edited clones from the HCP1324_c1A12 line were isolated by limiting dilution in 96 well plates coated with vitronectin. Cells were seeded at an average of 100 cells/well. 9600 iPSC were seeded in well A1 before 2× serial dilution in column 1 (A1-H1). Cells were then diluted 2× across all rows of the plate. Edited HCP1324_c1A12 were seed into mTeSR™ Plus. Media was exchanged every 2 days for complete mTeSR™ Plus. Cells were cultured for 12-14 days before screening wells for the presence of single colonies. Clones were expanded and genomic DNA isolated using QuickExtract™ (Lucigen) for screening by PCR in order to confirm targeted transgene insertion.

PCR reactions were performed across homology arm boundaries. The 5′ LHA boundary was amplified with primer 1824 (SEQ ID No.50) TAAGTTGGCTGTAAACAAAGTTGAA which is upstream of the 5′ homology arm and primer 1825 (SEQ ID No. 51)

GCACGTCGTCTCGGTAGC which is in the puromycin resistance gene The 3′ RHA boundary was amplified with the primer 2294 (SEQ ID No. 52) TCTACGAGAGGCCTGGGCTTC which is in the puromycin resistance gene and primer 2295 (SEQ ID No. 53) GTGGGGTGGCAGGTATTAGG which is in the right hand homology region of EEF1A1 exon 8.

PCR reactions were performed with Q5@ Hot Start High-Fidelity 2× Master Mix (NEB). PCR program denaturation 98° C., 30 s, 1 cycle, 40 cycles denaturation 98° C., 5 s, annealing 66° C., 10 s, Extension 72° C., 25 s, 1 cycle extension 72° C., 5 min. PCR products were gel purified and Sanger sequenced to confirm integration. Positive clones were expanded, and small cell banks produced. Maintenance of pluripotency marker expression was confirmed by flow cytometry. Four clones were progressed for differentiation, named 4B11, 5F4, 5H4 and 3D10.

RAG1 Exon 2 was targeted with guide RNA sequence AACATCTTCTGTCGCTGACT. This sequence corresponds to chromosome 11:36,574,494-36,574,513, build GRCh38/hg38.

### Nucleofection

CRISPR-Cas9 Ribonucleoprotein (RNP) complexes were formed prior to start of detachment of cells. Each nucleofection contained 52 pmol SpyFi-Cas9 (Aldevron) and 60 pmol of single-guide RNA targeting RAG1 (IDT) in DPBS at a final volume of 5 ul. Cells were detached using Accutase for 6 minutes at 37° C. and 2*10E5 (3E10) cells were used for each nucleofection in an Amaxa Nucleofector 4D. Cells were centrifuged, washed in DPBS and resuspended in 20 ul supplemented primary buffer P3. 5μ of RNPs was added, transferred to a slot in a 16-strip nucleocuvette and nucleofected using program CA-137. Cells were incubated for 3 min, 80 ul pre-warmed mTeSR-Plus/1xCloneR was added, incubated for another 1 min and then transferred to wells of a 12 well plate containing 1 ml per well mTeSR-Plus/1xCloneR.

### Single Cell Seeding and Clonal Expansion

Five days after nucleofection, cells were seeded in ten 96-well plates using Solentim VIPS™ and imaged at 2 hrs after seeding and for the first four days every day, then every other day with Solentim Cell Metric®. CloneR was present in the first three days of single-cell growth, at 1× concentrations for 24 hrs, then 0.25× for another 48 hrs. Plates were fed very other day. On days 13 and 14 after seeding, colonies that originated from a single cell, were detached with Versene and split into 12 well plates (40%, for expansion) and 96 well plates (60%, for genotyping). After two days, cells were genotyped using lon Torrent and only those with biallelic edits resulting in multiple premature stop codons were expanded into 6-well plates. Clones were subject to colony PCR using primers ATCCTGTGACATCTGCAACACT and ACAGCCCATCAATAATGCCAAC and cryopreserved after positive confirmation.

### T Cell Differentiation

HiPSC maintenance medium was removed, the cells washed twice with DMEM/F12 (Invitrogen). 2 mL of StemPro34 PLUS (StemPro34 from Invitrogen; StemPro34 basal media, with supplement added and Penicillin Streptomycin (1% v/v: Invitrogen) and Glutamine (2 mM: Invitrogen), Ascorbic Acid (50 mg/ml: Sigma Aldrich) and monothioglycerol (100 mM: Sigma Aldrich), further supplemented with 50 ng/ml of Activin A (Miltenyi Biotec) was added and incubated for 4 hours. Volumes are dependent of culture flask size, typically at least 2 mls/9 cm², and 20 mls/150 cm².

After 4 hours, the medium was removed, and the cells washed twice with DMEM/F12 to remove residual high concentration Activin A. The medium was replaced with 2 mL of StemPro34 PLUS supplemented with 5 ng/ml of Activin A, 10 ng/ml of BMP4 (Miltenyi Biotec) and 5 ng/ml of bFGF (Miltenyi Biotec) and incubated for 44 hours (Stage 1 media).

The medium was then replaced with fresh Stage 1 media and supplemented with 10 μM CHIR-99021 (Selleckchem) and further cultured for 48 hours.

On Day 4, the medium was removed, and the cells washed twice with DMEM/F12 to remove residual stage 1 cytokines. The medium was then replaced with StemPro34 PLUS supplemented with 100 ng/ml of SCF (Miltenyi Biotec) and 15 ng/ml of VEGF (Miltenyi Biotec) and incubated for 48 hours (Stage 2 media). The medium was then replenished with fresh Stage 2 media and the cells cultured for a further 48 hours.

The medium was then replaced by the Stage 3 medium which requires the minimum of SCF and VEGF and the cells cultured for between 16-18 days, with demi depletion feeding every 48 h. Typically this involved harvest of media and collection of cells in suspension by centrifugation (300 g, 10 min), and returning suspension cells to culture with fresh media (i.e 20 ml for a T150 flask).

On approximately day 16 CD34+ cells were isolated from resulting monolayers for onward culture. CD34+ cells were harvested by sequential incubation with Accutase (STEMCELL Technologies) for 30 mins at 37° C. and then Collagenase II (Invitrogen: 2 mg/ml) for 30 mins at 37° C. Cell suspensions were collected and washed (×2 centrifugation at 300 g for 12 min in DMEM/F12), prior to CD34+ cell isolation via Magnetic activated beads (MACS) isolation (Miltenyi: according to manufacturer's instructions).

For continued lymphoid proliferation and differentiation, STEMCELL Technologies proprietary 2 stage (Lymphoid Proliferation/T cell Maturation) media was employed.

During culture in STEMCELL Technologies Lymphoid Proliferation media T-cell progenitors non-edited control lines were lentivirally transduced with the ADP A2M4 TCR in the presence of poloxamer F108 (Sigma).

### T Cell Phenotyping

iPSC derived T Cells were phenotyped using flow cytometry. The cells were stained with CD3 (clone SK7); A2M4 dextramer and live/dead stain EF506 to show expression of ADP A2M4 TCR in iPSC derived T Cells.

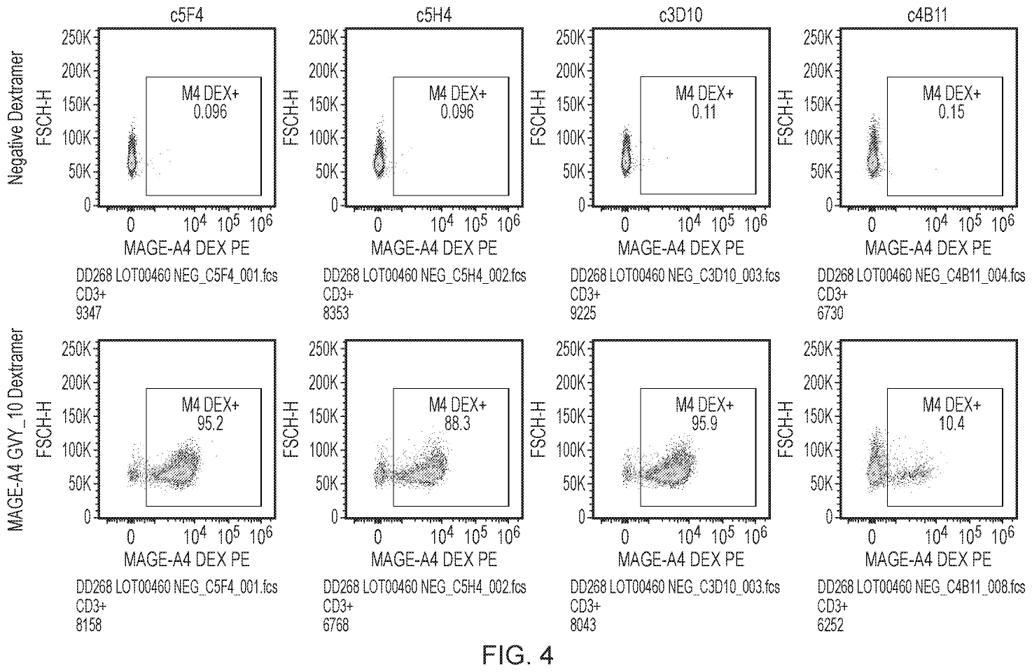

Expression of ADP A2M4 in iT cells can be induced via lentiviral transduction of CD7+CD5+ progenitors as described above. The iPSC line HCP1324_c1A 12 was differentiated and transduced with lentivirus express containing the ADP A2M4 T cell at Stage 4 (CD7+CD5+ progenitor) and the transduced cells were progressed through the differentiation protocol. Cells were phenotyped by FACS and ADP A2M4 expression measured by staining with anti-TCR antibody anti-Vα24 and MAGE-A4 GVYDGREHTV/HLA-A*0201 dextramer. Differentiation was found to be unaffected by editing. In addition, knock-in of ADP A2M4 at a single allele is sufficient to promote ADP A2M4 expression. (FIG. 4).

### Flow Cytometry Staining

The following antibodies were used for flow cytometry: CD8αβ-BV650 (clone2ST8.5H7), TCRγδ-APC (clone B1), TCR Vα24-PE (Clone IP26), CD3-APC-R700 (clone UCHT1), CD4-BV605 (clone RPA-T4), CD8α-PE-CY7 (clone RPA-T8), CD69-BUV395 (clone FN50), CD25-BV421 (Clone BC96), HLA-DR-AF488 (clone L243), and EF506-BV510. Samples were acquired on the BD Fortessa.

### KILR Cytotoxicity Assay

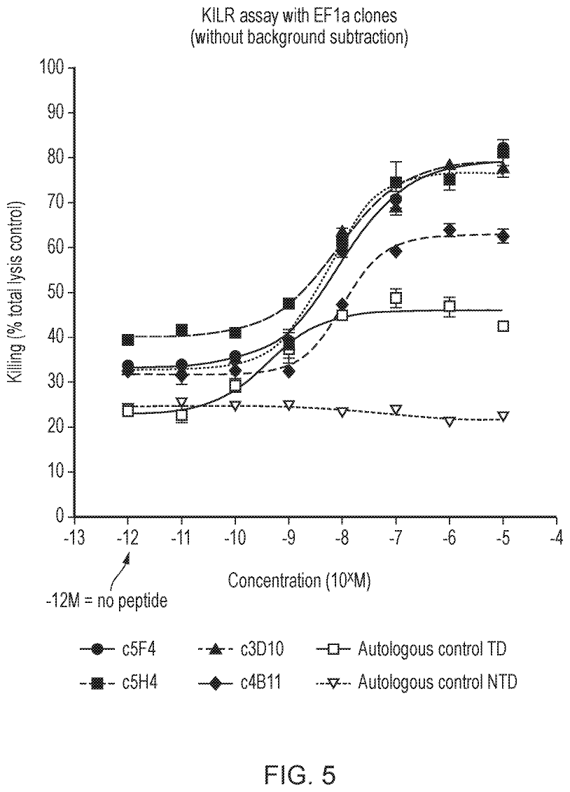

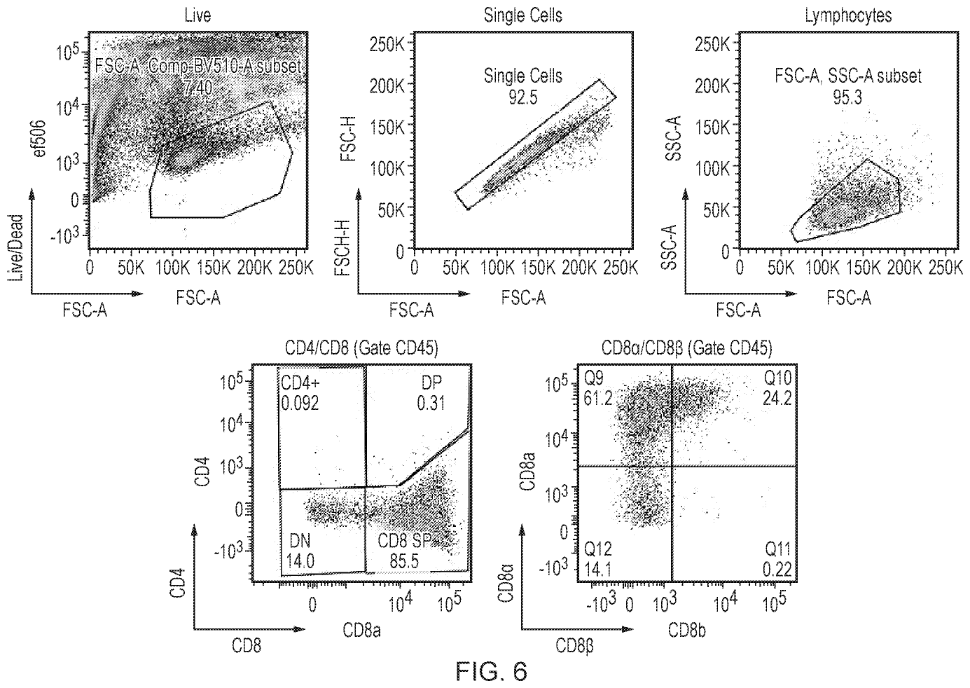

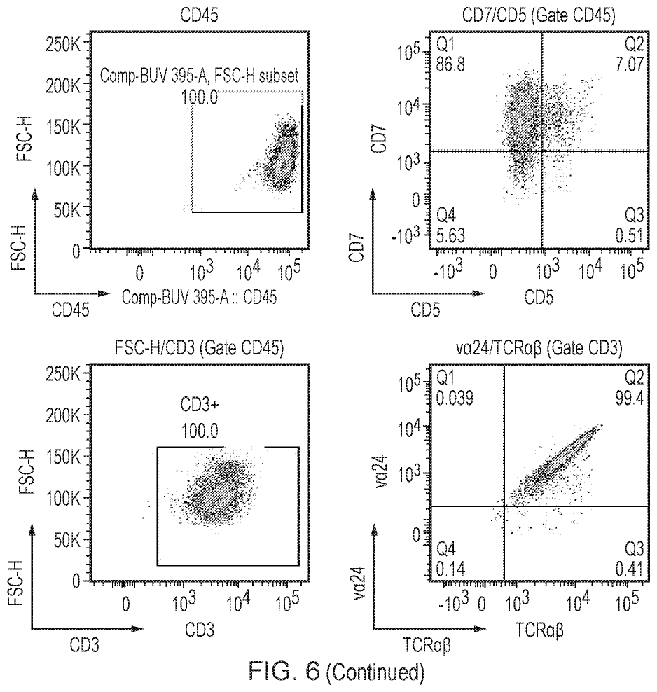

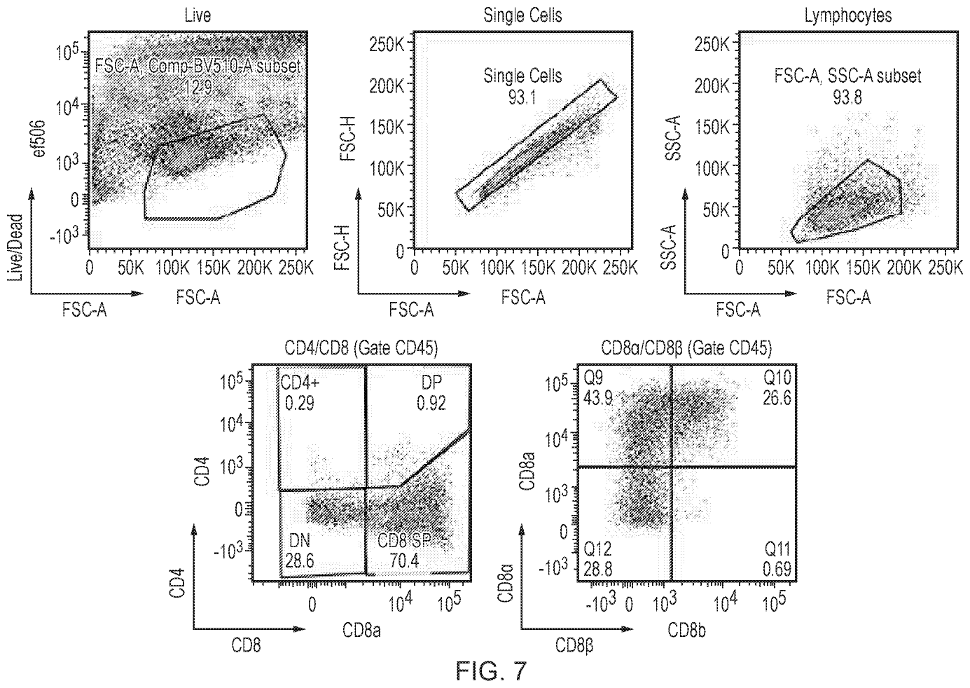

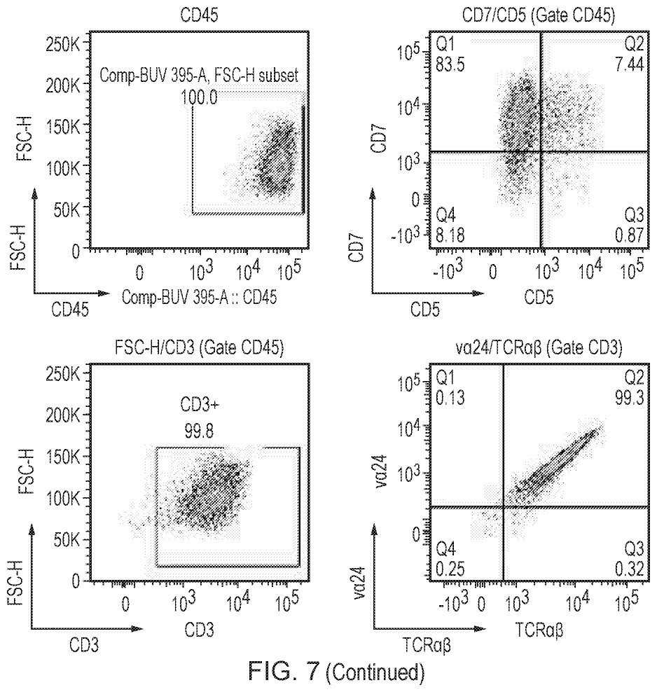

10,000 KILR T2s (generated by transduction with KILR retroparticles (DiscoverX)) were added per well of a 384 well white LUMITRAC 600 microplate (Greiner). T cells derived from iPSC lines at the end of stage 6 were added at 20,000 cells per well. MAGE-A4 peptide (GVYDGREHTV) was added to cells in a titration from 10−5M to 10−11M. Cells and peptide were co-cultured for 24 hours at 37° C. under normoxia before addition of KILR detection solution (KILR Detection Kit (DiscoverX)) for 1 hr at room temperature. Luminescence from samples was detected using the FLUOstar Omega Microplate Reader. iT cells differentiated from edited iPSC clones were found to exhibit specific and potent cytolytic effector function (FIG. 5).

### Incucyte® Cytotoxicity Assay

GFPtive A375 (MAGE-A4^{+ive}, HLA-A2*02^{+ive}) target cells were seeded in 384 well plates (Greiner Bio-one) at a density of 1500/well in 20 μl of complete RPMI 1640 (RPMI 1640, 10% (v/v) FCS, 100 U/ml penicillin, 100 μg/ml streptomycin and 2 mM L-Glutamine). Plates were added to the Incucyte Zoom and incubated at 37° C, with 5% CO₂ in a humidified incubator. 24 hours later, targets were either pulsed with MAGE-A4 peptide (GVYDGREHTV) at a final concentration of 10 μM or left without peptide. 6000 iT cells were added in triplicate in 10 μl media. Images were taken every 2 hours, for 6 days in the Incucyte. Data was then analysed using ZOOM2018A software using top hat process definitions.

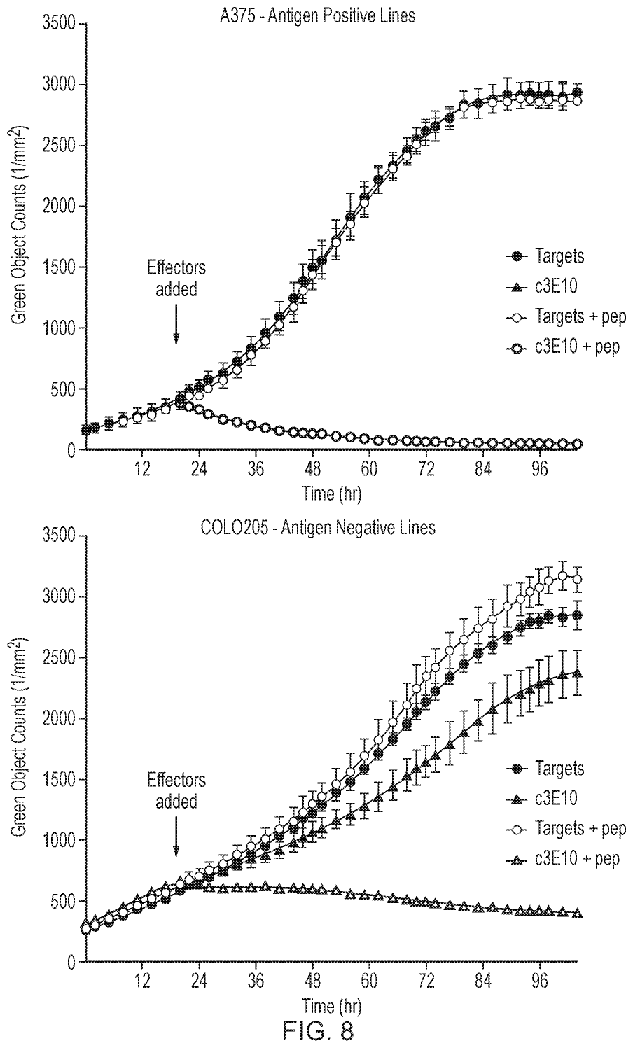

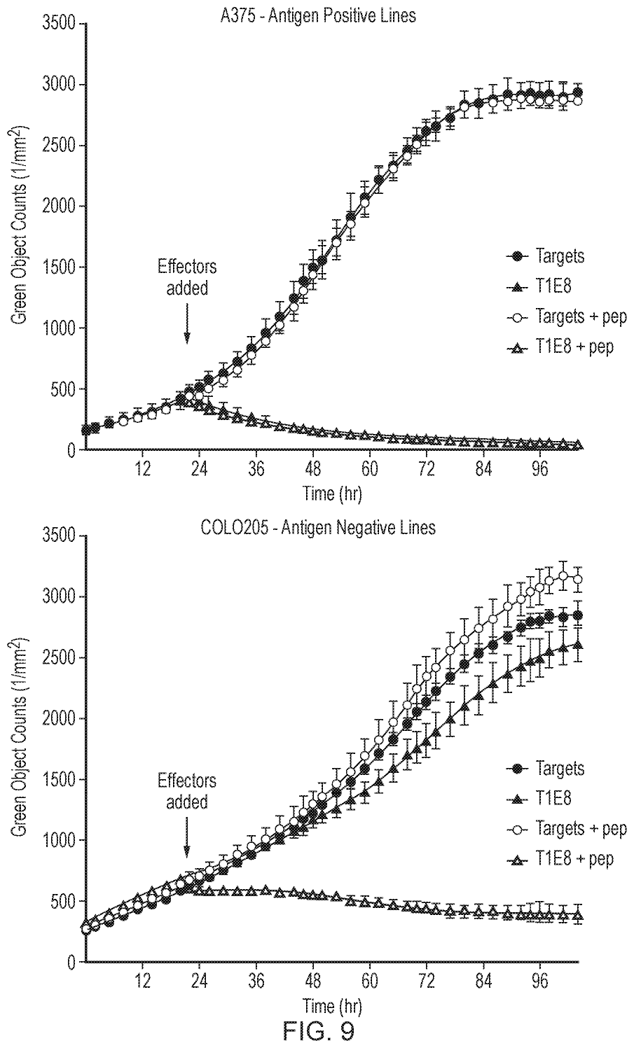

EEF1α^{+/A2M4} iT cells (c3E10) and RAG^{-/-}EEF1α^{+/A2M4} iT cells (T1E8) were found to induce apoptosis in both the presence or absence of MAGE-A4 derived peptide GVYDGREHTV in MAGEA4+cells, but to only induce apoptosis in MAGEA4⁻ cells in the presence of the MAGE-A4 derived peptide (FIGS. 8 and 9).

### CONCLUSION

We have presented a novel editing strategy that has enabled the differentiation of ADP A2M4 TCR expressing iT-cells. Edited ADP A2M4 TCR iT-cell activation and cytolytic effector function has been shown to be antigen dependent. The phenotype of edited ADP

A2M4 iT-cells including CD8αα expression, potent cytokine and cytotoxic activity suggests that edited ADP A2M4 iT-cells possess an innate and adaptive like phenotype.

Although dextramer staining suggests that edited ADP A2M4 iT-cells only express the ADP-A2M4 TCR, the technology is applicable to a full TCR repertoire. The edited A2M4 TCR iT-cells exhibit potent cytolytic and effector function, which is comparable or increased compared to A2M4 TCR transduced PBL from healthy donors. This suggests that, like autologous A2M4 TCR T-cells, ADP-A2M4 iT-cells will be effective in cell therapy and bring clinical benefit to patients.

The generation of ADP-A2M4 iT-cells is an important milestone in producing an iPSC derived allogeneic platform. The ability to promote TCR expression in iT-cells via genetic knock-in at a single, defined locus offers an opportunity to produce multiple clonal iPSC banks encoding specific TCRs against a range of tumour antigens. This “off-the-shelf” platform may deliver a range of defined and consistent T-cell therapies to patients specific to their tumour antigen expression profile in a timely manner.

Sequences
MAGE A4
SEQ ID NO: 1
MSSEQKSQHC KPEEGVEAQE EALGLVGAQA PTTEEQEAAV SSSSPLVPGT
LEEVPAAESA GPPQSPQGAS ALPTTISFTC WRQPNEGSSS QEEEGPSTSP
DAESLFREAL SNKVDELAHF LLRKYRAKEL VTKAEMLERV IKNYKRCFPV
IFGKASESLK MIFGIDVKEV DPASNTYTLV TCLGLSYDGL LGNNQIFPKT
GLLIIVLGTI AMEGDSASEE EIWEELGVMG VYDGREHTVY GEPRELLTQD
WVQENYLEYR QVPGSNPARY EFLWGPRALA ETSYVKVLEH VVRVNARVRI
AYPSLREAAL LEEEEGV,
MAGE A4 peptide
SEQ ID NO: 2
GVYDGREHTV,
(CD8α) CDRs bold underlined, signal sequence italic underlined
SEQ ID NO: 3
MALPVTALLLPLALLLHAARPSQFRVSPLDRTWNLGETVELKCQVLLSNPTSGCSWLFQPRGAAASPTF
LLYLSQNKPKAAEGLDTQRFSGKRLGDTFVLTLSDFRRENEGYYFCSALSNSIMYFSHFVPVFLPAKPT
TTPAPRPPTPAPTIASQPLSLRPEACRPAAGGAVHTRGLDFACDIYIWAPLAGTCGVLLLSLVITLYCN
HRNRRRVCKCPRPVVKSGDKPSLSARYV;
(CD8α)
SEQ ID NO: 4
ATGGCCTTACCAGTGACCGCCTTGCTCCTGCCGCTGGCCTTGCTGCTCCACGCCGCCAGGCCGAGCCAG
TTCCGGGTGTCGCCGCTGGATCGGACCTGGAACCTGGGCGAGACAGTGGAGCTGAAGTGCCAGGTGCTG
CTGTCCAACCCGACGTCGGGCTGCTCGTGGCTCTTCCAGCCGCGCGGCGCCGCCGCCAGTCCCACC
TTCCTCCTATACCTCTCCCAAAACAAGCCCAAGGCGGCCGAGGGGCTGGACACCCAGCGGTTCTCGGGC
AAGAGGTTGGGGGACACCTTCGTCCTCACCCTGAGCGACTTCCGCCGAGAGAACGAGGGCTACTATTTC
TGCTCGGCCCTGAGCAACTCCATCATGTACTTCAGCCACTTCGTGCCGGTCTTCCTGCCAGCGAAGCCC
ACCACGACGCCAGCGCCGCGACCACCAACACCGGCGCCCACCATCGCGTCGCAGCCCCTGTCCCTGCGC
CCAGAGGCGTGCCGGCCAGCGGCGGGGGGCGCAGTGCACACGAGGGGGCTGGACTTCGCCTGTGAT
ATCTACATCTGGGCGCCCTTGGCCGGGACTTGTGGGGTCCTTCTCCTGTCACTGGTTATCACCCTTTAC
TGCAACCACAGGAACCGAAGACGTGTTTGCAAATGTCCCCGGCCTGTGGTCAAATCGGGAGACAAGCCC
AGCCTTTCGGCGAGATACGTCGGTTCAAGAGCTAAAAGAAGTGGTAGTGGTGCCCCTGTGA;
(MAGE A4 TCR α chain) CDRs bold underlined
SEQ ID NO: 5
MKKHLTTFLVILWLYFYRGNGKNQVEQSPQSLIILEGKNCTLQCNYTVSPFSNLRWYKQDTGRGPVSLT
ILTFSENTKSNGRYTATLDADTKQSSLHITASQLSDSASYICVVSGGTDSWGKLQFGAGTQVVVTPDIQ
NPDPAVYQLRDSKSSDKSVCLFTDFDSQTNVSQSKDSDVYITDKTVLDMRSMDFKSNSAVAWSNKSDFA
CANAFNNSII PEDT FFPSPESSCDVKLVEKSFETDTNLNFQNLSVIGFRILLLKVAGENLLMTLRL
WSSGSRAKR;
(MAGE A4 TCR chain coding sequence)
SEQ ID NO: 6
ATGAAGAAGCACCTGACCACCTTTCTCGTGATCCTGTGGCTGTACTTCTACCGGGGCAACGGCAAGA
ACCAGGTGGAACAGAGCCCCCAGAGCCTGATCATCCTGGAAGGCAAGAACTGCACCCTGCAGTGCAA
CTACACCGTGTCCCCCTTCAGCAACCTGCGGTGGTACAAGCAGGACACCGGCAGAGGCCCTGTGTCCCTGA
CCATCCTGACCTTCAGCGAGAACACCAAGAGCAACGGCCGGTACACCGCCACCCTGGACGCCGATACAAAG
CAGAGCAGCCTGCACATCACCGCCAGCCAGCTGAGCGATAGCGCCAGCTACATCTGCGTGGTGTCCGGCGG
CACAGACAGCTGGGGCAAGCTGCAGTTTGGCGCCGGAACACAGGTGGTCGTGACCCCCGACATCCAGAAC
CCTGACCCTGCCGTGTACCAGCTGCGGGACAGCAAGAGCAGCGACAAGAGCGTGTGCCTGTTCACCGA
CTTCGACAGCCAGACCAACGTGTCCCAGAGCAAGGACAGCGACGTGTACATCACCGACAAGACCGTGCTGG
ACATGCGGAGCATGGACTTCAAGAGCAATAGCGCCGTGGCCTGGTCCAACAAGAGCGACTTCGCCTGCGCC
AACGCCTTCAACAACAGCATTATCCCCGAGGACACATTCTTCCCAAGCCCCGAGAGCAGCTGCGACGTCAA
GCTGGTGGAAAAGAGCTTCGAGACAGACACCAACCTGAACTTCCAGAACCTGAGCGTGATCGGCTTCAGA
ATCCTGCTGCTGAAGGTGGCCGGCTTCAACCTGCTGATGACCCTGAGACTGTGGTCCAGCGGCAGCC
GGGCCAAGAGA;
(MAGE A4 TCR β chain) CDRs bold underlined
SEQ ID NO: 7
MASLLFFCGAFYLLGTGSMDADVTQTPRNRITKTGKRIMLECSQTKGHDRMYWYRQDPGLGLRLIYY
SFDVKDINKGEISDGYSVSRQAQAKFSLSLESAIPNQTALYFCATSGQGAYEEQFFGPGTRLTVLED
LKNVFPPEVAVFEPSEAEISHTQKATLVCLATGFYPDHVELSWWVNGKEVHSGVSTDPQPLKEQPALNDSR
YCLSSRLRVSATFWQNPRNHFRCQVQFYGLSENDEWTQDRAKPVTQIVSAEAWGRADCGFTSESYQQ
GVLSATILYEILLGKATLYAVLVSALVLMAMVKRKDSRG;
(MAGE A4 TCR β chain coding sequence)
SEQ ID NO: 8
ATGGCCAGCCTGCTGTTCTTCTGCGGCGCCTTCTACCTGCTGGGCACCGGCTCTATGGATGCCGACGTG
ACCCAGACCCCCCGGAACAGAATCACCAAGACCGGCAAGCGGATCATGCTGGAATGCTCCCAGACCAAG
GGCCACGACCGGATGTACTGGTACAGACAGGACCCTGGCCTGGGCCTGCGGCTGATCTACTACAGCTTC
GACGTGAAGGACATCAACAAGGGCGAGATCAGCGACGGCTACAGCGTGTCCAGACAGGCTCAGGCCAAG
TTCAGCCTGTCCCTGGAAAGCGCCATCCCCAACCAGACCGCCCTGTACTTTTGTGCCACAAGCGGCCAG
GGCGCCTACGAGGAGCAGTTCTTTGGCCCTGGCACCCGGCTGACAGTGCTGGAAGATCTGAAGAACGTG
TTCCCCCCAGAGGTGGCCGTGTTCGAGCCTTCTGAGGCCGAAATCAGCCACACCCAGAAAGCCACACTC
GTGTGTCTGGCCACCGGCTTCTACCCCGACCACGTGGAACTGTCTTGGTGGGTCAACGGCAAAGAG
GTGCACAGCGGCGTGTCCACCGATCCCCAGCCTCTGAAAGAACAGCCCGCCCTGAACGACAGCCGGTAC
TGCCTGAGCAGCAGACTGAGAGTGTCCGCCACCTTCTGGCAGAACCCCAGAAACCACTTCAGATGCCAG
GTGCAGTTTTACGGCCTGAGCGAGAACGACGAGTGGACCCAGGACAGAGCCAAGCCCGTGACACAGATC
GTGTCTGCCGAAGCTTGGGGGCGCGCCGATTGTGGCTTTACCAGCGAGAGCTACCAGCAGGGCGTGCTG
AGCGCCACCATCCTGTACGAGATCCTGCTGGGAAAGGCCACACTGTACGCCGTGCTGGTGTCTGCC
CTGGTGCTGATGGCCATGGTCAAGCGGAAGGACAGCCGGGGC;
(MAGE A4 TCR α chain variable region) 136AA-CDRs bold underlined
SEQ ID NO: 9
MKKHLTTFLVILWLYFYRGNGKNQVEQSPQSLIILEGKNCTLQCNYTVSPFSNLRWYKQDTGRGPVSL
TILTFSENTKSNGRYTATLDADTKQSSLHITASQLSDSASYICVVSGGTDSWGKLQFGAGTQVVVTPD;
(MAGE A4 TCR β chain variable region) 133AA-CDRs bold underlined
SEQ ID NO: 10
MASLLFFCGAFYLLGTGSMDADVTQTPRNRITKTGKRIMLECSQTKGHDRMYWYRQDPGLGLRLIYYS
FDVKDINKGEISDGYSVSRQAQAKFSLSLESAIPNQTALYFCATSGQGAYEEQFFGPGTRLTVLE;
CDR1 MAGE A4 TCR α chain, (residues 48-53)
SEQ ID NO: 11
VSPFSN;
CDR2 MAGE A4 TCR α chain, (residues 71-76)
SEQ ID NO: 12
LTFSEN;
CDR3 MAGE A4 TCR α chain, (residues 111-125)
SEQ ID NO: 13
CVVSGGTDSWGKLQF;
CDR1 MAGE A4 TCR β chain, (residues 46-50)
SEQ ID NO: 14
KGHDR;
CDR2 MAGE A4 TCR β chain, (residues 68-73)
SEQ ID NO: 15
SFDVKD;
CDR3 MAGE A4 TCR β chain, (residues 110-123)
SEQ ID NO: 16
CATSGQGAYEEQFF;
CDR1 CD8α (residues 45-53)
SEQ ID NO: 17
VLLSNPTSG
CDR2 CD8α (residues 72-79)
SEQ ID NO: 18
YLSQNKPK
CDR3 CD8α (residues 118-123)
SEQ ID NO: 19
LSNSIM
Human Alpha-fetoprotein-TCR epitope bold underlined
SEQ ID NO: 20
MKWVESIFLI FLLNFTESRT LHRNEYGIAS ILDSYQCTAE ISLADLATIF FAQFVQEATY
KEVSKMVKDA LTAIEKPTGD EQSSGCLENQ LPAFLEELCHEKEILEKYGH SDCCSQSEEG
RHNCFLAHKK PTPASIPLFQ VPEPVTSCEAYEEDRETFMN KFIYEIARRH PFLYAPTILL
WAARYDKIIP SCCKAENAVE CFQTKAATVT KELRESSLLN QHACAVMKNF GTRTFQAITV
TKLSQKFTKV NFTEIQKLVL DVAHVHEHCC RGDVLDCLQD GEKIMSYICS QQDTLSNKIT
ECCKLTTLER GQCIIHAEND EKPEGLSPNL NRFLGDRDEN QFSSGEKNIF LASFVHEYSR
RHPQLAVSVI LRVAKGYQEL LEKCFQTENP LECQDKGEEELQKYIQESQA LAKRSCGLFQ
KLGEYYLQNA FLVAYTKKAP QLTSSELMAITRKMAATAAT CCQLSEDKLL ACGEGAADII
IGHLCIRHEM TPVNPGVGQCCTSSYANRRP CFSSLVVDET YVPPAFSDDK FIFHKDLCQA
QGVALQTMKQ EFLINLVKQK PQITEEQLEA VIADFSGLLE KCCQGQEQEV CFAEEGQKLI
SKTRAALGV
Human Alpha-fetoprotein peptide
SEQ ID NO: 21
FMNKFIYEI
Parental AFP TCR TRAV12-2*02/TRAJ41*01/TRAC alpha chain amino
acid extracellular sequence
SEQ ID NO: 22
Q K E V E Q N S G P L S V P E G A I A S L N C T Y S D
R G S Q S F F W Y R Q Y S G K S P E L I M S I Y S N G
D K E D G R E T A Q L N K A S Q Y V S L L I R D S Q P
S D S A T Y L C A V N S D S G Y A L N F G K G T S L L
V T P H I Q N P D P A V Y Q L R D S K S S D K S V C L
F T D F D S Q T N V S Q S K D S D V Y I T D K T V L D
M R S M D F K S N S A V A W S N K S D F A C A N A F N
N S I I P E D T F F P S P E S S
Parental AFP TCR TRBV9*01/TRBD2/TRBJ2-7*01/TRBC2 beta chain
aminoacid extracellular sequence
SEQ ID NO: 23
D S G V T Q T P K H L I T A T G Q R V T L R C S P R S
G D L S V Y W Y Q Q S L D Q G L Q F L I Q Y Y N G E E
R A K G N I L E R F S A Q Q F P D L H S E L N L S S L
E L G D S A L Y F C A S S L G G E S E Q Y F G P G T R
L T V T E D L K N V F P P E V A V F E P S E A E I S H
T Q K A T L V C L A T G F Y P D H V E L S W W V N G K
E V H S G V S T D P Q P L K E Q P A L N D S R Y C L S
S R L R V S A T F W Q N P R N H F R C Q V Q F Y G L S
E N D E W T Q D R A K P V T Q I V S A E A W G R A D
Reference TCR alpha chain DNA sequence
SEQ ID NO: 24
caaaaagaagttgagcagaattctggacccctcagtgttccagagggagccattgcctctctcaactgc
acttacagtgaccgaggttcccagtccttcttctggtacagacaatattctgggaaaagccctgagttg
ataatgtccatatactccaatggtgacaaagaagatggaaggtttacagcacagctcaataaagccagc
cagtatgtttctctgctcatcagagactcccagcccagtgattcagccacctacctctgtgccgtgaat
agtgattccgggtatgcactcaacttcggcaaaggcacctcgctgttggtcacaccccatatccag
aaccctgaccctgccgtgtaccagctgagagactctaagtcgagtgacaagtctgtctgcctattcacc
gattttgattctcaaacaaatgtgtcacaaagtaaggattctgatgtgtatatcacagacaaatgtgtg
ctagacatgaggtctatggacttcaagagcaacagtgtgtggcctggagcaacaaatctgactttgca
tgtgcaaacgccttcaacaacagcattattccagaagacaccttcttccccagcccagaaagttcc
Reference TCR beta chain DNA sequence
SEQ ID NO: 25
gattctggagtcacacaaaccccaaagcacctgatcacagcaactggacagcgagtgacgct
gagatgctcccctaggtctggagacctctctgtgtactggtaccaacagagcctggaccagg
gcctccagttcctcattcagtattataatggagaagagagagcaaaaggaaacattctt
gaacgattctccgcacaacagttccctgacttgcactctgaactaaacctgagctctctgg
agctgggggactcagctttgtatttctgtgccagcagcctcgggggggaatctgagcagtac
ttcgggccgggcaccaggctcacggtcacagaggacctgaaaaacgtgttcccacccgaggt
cgctgtgtttgagccatcagaagcagagatctcccacacccaaaaggccacactggtgtgcc
tggccaccggtttctaccccgaccacgtggagctgagctggtgggtgaatgggaaggaggt
gcacagtggggtctgcacagacccgcagcccctcaaggagcagcccgccctcaatgact
ccagatacgctctgagcagccgcctgagggtctcggccaccttctggcaggacccccgcaac
cacttccgctgtcaagtccagttctacgggctctcggagaatgacgagtggacccaggatag
ggccaaacccgtcacccagatcgtcagcgccgaggcctggggtagagcagac
SEQ ID NO: 26
DRGSQS (αCDR1),
SEQ ID NO: 27
TYSNGD (αCDR2),
SEQ ID NO: 28
AVNSDSGYALNF (αCDR3),
SEQ ID NO: 29
SGDLS (βCDR1),
SEQ ID NO: 30
YYNGEE (βCDR2),
SEQ ID NO: 31
ASSLGGESEQY (βCDR3),
SEQ ID NO: 32
DRGSQA (αCDR1),
SEQ ID NO: 33
AVNSDSSYALNF (αCDR3),
SEQ ID NO: 34
AVNSDSGVALNE (αCDR3),
SEQ ID NO: 35
AVNSQSGYALNF (αCDR3),
SEQ ID NO: 36
AVNSQSGYSLNF (αCDR3),
SEQ ID NO: 37
AVNSQNGYALNF (αCDR3),
SEQ ID NO: 38
DRGSFS (αCDR1),
SEQ ID NO: 39
DRGSYS (αCDR1),
SEQ ID NO: 40
AVNSQSSYALNF (αCDR3),
Variant AFP TCR (AFP TRAV12-2*02/TRAJ41*01/TRAC alpha chain
amino acid extracellular sequence, CDRs underlined
SEQ ID NO: 41
QKEVEQNSGPLSVPEGAIASLNCTYSDRGSQAFFWYRQYSGKSPELIMSIYSNGDKEDGRFTAQLNKAS
QYVSLLIRDSQPSDSATYLCAVNSQSGYALNFGKGTSLLVTPHIQNPDPAVYQLRDSKSSDKSVCLFTD
FDSQTNVSQSKDSDVYITDKTVLDMRSMDFKSNSAVAWSNKSDFACANAFNNSIIPEDTFFPSPESS
Variant AFP TCR TRBV9*01/TRBD2/TRBJ2-7*01/TRBC2 beta chain amino
acid extracellular sequence, CDRs underlined
SEQ ID NO: 42
DSGVTQTPKHLITATGQRVTLRCSPRSGDLSVYWYQQSLDQGLQFLIQYYNGEERAKGNILERFSAQQFPD
LHSELNLSSLELGDSALYFCASSLGGESEQYFGPGTRLTVTEDLKNVFPPEVAVFEPSEAEISHTQKATLV
CLATGFYPDHVELSWWVNGKEVHSGVSTDPQPLKEQPALNDSRYCLSSRLRVSATFWQNPRNHERCQVQF
YGLSENDEWTQDRAKPVTQIVSAEAWGRAD
Left Homology Arm (LHA)
SEQ ID NO: 43
AGTTGATAGAGTACTGTCTGCCTTCATAGGTATTTAGTATGCTGTAAATATTTTTAGGTATTGGT
ACTGTTCCTGTTGGCCGAGTGGAGACTGGTGTTCTCAAACCCGGTATGGTGGTCACCTTTGCTCC
AGTCAACGTTACAACGGAAGTAAAATCTGTCGAAATGCACCATGAAGCTTTGAGTGAAGCTCTTC
CTGGGGACAATGTGGGCTTCAATGTCAAGAATGTGTCTGTCAAGGATGTTCGTCGTGGCAACGTT
GCTGGTGACAGCAAAAATGACCCACCAATGGAAGCAGCTGGCTTCACTGCTCAGGTAACAATTTA
AAGTAACATTAACTTATTGCAGAGGCTAAAGTCATTTGAGACTTTGGATTTGCACTGAATGCAAA
TCTTTTTTCCAAGGTGATTATCCTGAACCATCCAGGCCAAATAAGCGCCGGCTATGCCCCTGTAT
TGGATTGCCACACGGCTCACATTGCATGCAAGTTTGCTGAGCTGAAGGAAAAGATTGATCGCCGT
TCTGGTAAAAAGCTGGAAGATGGCCCTAAATTCTTGAAGTCTGGTGATGCTGCCATTGTTGATAT
GGTTCCTGGCAAGCCCATGTGTGTTGAGAGCTTCTCAGACTATCCACCTTTGGGTAAGGATGACT
ACTTAAATGTAAAAAAGTTGTGTTAAAGATGAAAAATACAACTGAACAGTACTTTGGGTAATAAT
TAACTTTTTTTTTAATAGGTCGCTTTGCTGTTCGTGATATGAGACAGACAGTTGCGGTGGGTGTC
ATCAAAGCAGTGGACAAGAAGGCTGCTGGAGCTGGCAAGGTCACCAAGTCTGCCCAGAAAGCTCA
GAAGGCTAAA
Right Homology Arm (RHA)
SEQ ID NO: 44
TGAATATTATCCCTAATACCTGCCACCCCACTCTTAATCAGTGGTGGAAGAACGGTCTCAGAACTGT
TTGTTTCAATTGGCCATTTAAGTTTAGTAGTAAAAGACTGGTTAATGATAACAATGCATCGTAAAAC
CTTCAGAAGGAAAGGAGAATGTTTTGTGGACCACTTTGGTTTTCTTTTTTGCGTGTGGCAGTTTTAA
GTTATTAGTTTTTAAAATCAGTACTTTTTAATGGAAACAACTTGACCAAAAATTTGTCACAGAATTT
TGAGACCCATTAAAAAAGTTAAATGAGAAACCTGTGTGTTCCTTTGGTCAACACCGAGACATTTAGG
TGAAAGACATCTAATTCTGGTTTTACGAATCTGGAAACTTCTTGAAAATGTAATTCTTGAGTTAACA
CTTCTGGGTGGAGAATAGGGTTGTTTTCCCCCCACATAATTGGAAGGGGAAGGAATATCATTTAAAG
CTATGGGAGGGTTGCTTTGATTACAACACTGGAGAGAAATGCAGCATGTTGCTGATTGCCTGTCACT
AAAACAGGCCAAAAACTGAGTCCTTGTGTTGCATAGAAAGCTTCATGTTGCTAAACCAATGTTAAGT
GAATCTTTGGAAACAAAATGTTTCCAAATTACTGGGATGTGCATGTTGAAACGTGGGTTAAAATGAC
TGGGCAGTGAAAGTTGACTATTTGCCATGACATAAGAAATAAGTGTAGTGGCTAGTGTACACCCTAT
GAGTGGAAGGGTCCATTTTGAAGTCAGTGGAGTAAGCTTTATGCCAGTTTGATGGTTTCACAAGTTC
TATTGAGTGCTATTCAGAATAGGAACAAGGTTCTAATAGAAAAAGATGGCAATTTGAAGTAGCTATA
AAATTAGACTAATCTACATTGCTTTTCTCCTGCAGAGTCTAATACCTTTTATGCTTTGATAATTAGC
AGTTTGTCTAC
FuT2A_A2M4
SEQ ID NO: 45
TCCAGCGGCAGCCGGGCCAAGAGATCTGGATCAGGTGAGGGCAGAGGCAGCCTGCTGACATGTGGCGAC
GTGGAAGAAAACCCTGGCCCTATGAAGAAGCACCTGACCACCTTTCTCGTGATCCTGTGGCTGTACTTC
TACCGGGGCAACGGCAAGAACCAGGTGGAACAGAGCCCCCAGAGCCTGATCATCCTGGAAGGCAAG
AACTGCACCCTGCAGTGCAACTACACCGTGTCCCCCTTCAGCAACCTGCGGTGGTACAAGCAGGACACC
GGCAGAGGCCCTGTGTCCCTGACCATCCTGACCTTCAGCGAGAACACCAAGAGCAACGGCCGGTACACC
GCCACCCTGGACGCCGATACAAAGCAGAGCAGCCTGCACATCACCGCCAGCCAGCTGAGCGATAGCGCC
AGCTACATCTGCGTGGTGTCCGGCGGCACAGACAGCTGGGGCAAGCTGCAGTTTGGCGCCGGAACACAG
GTGGTCGTGACCCCCGACATCCAGAACCCTGACCCTGCCGTGTACCAGCTGCGGGACAGCAAGAGC
AGCGACAAGAGCGTGTGCCTGTTCACCGACTTCGACAGCCAGACCAACGTGTCCCAGAGCAAGGACAGC
GACGTGTACATCACCGACAAGACCGTGCTGGACATGCGGAGCATGGACTTCAAGAGCAATAGCGCCGTG
GCCTGGTCCAACAAGAGCGACTTCGCCTGCGCCAACGCCTTCAACAACAGCATTATCCCCGAGGACACA
TTCTTCCCAAGCCCCGAGAGCAGCTGCGACGTCAAGCTGGTGGAAAAGAGCTTCGAGACAGACACCAAC
CTGAACTTCCAGAACCTGAGCGTGATCGGCTTCAGAATCCTGCTGCTGAAGGTGGCCGGCTTCAACCTG
CTGATGACCCTGAGACTGTGGTCCAGCGGCAGCCGGGCCAAGAGATCTGGATCCGGCGCTACCAACTTT
AGCCTGCTGAAGCAGGCCGGGGACGTGGAAGAAAACCCTGGCCCTAGGATGGCCAGCCTGCTGTTCTTC
TGCGGCGCCTTCTACCTGCTGGGCACCGGCTCTATGGATGCCGACGTGACCCAGACCCCCCGGAACAGA
ATCACCAAGACCGGCAAGCGGATCATGCTGGAATGCTCCCAGACCAAGGGCCACGACCGGATGTAC
TGGTACAGACAGGACCCTGGCCTGGGCCTGCGGCTGATCTACTACAGCTTCGACGTGAAGGACATCAAC
AAGGGCGAGATCAGCGACGGCTACAGCGTGTCCAGACAGGCTCAGGCCAAGTTCAGCCTGTCCCTGGAA
AGCGCCATCCCCAACCAGACCGCCCTGTACTTTTGTGCCACAAGCGGCCAGGGCGCCTACGAGGAGCAG
TTCTTTGGCCCTGGCACCCGGCTGACAGTGCTGGAAGATCTGAAGAACGTGTTCCCCCCAGAGGTGGCC
GTGTTCGAGCCTTCTGAGGCCGAAATCAGCCACACCCAGAAAGCCACACTCGTGTGTCTGGCCACC
GGCTTCTACCCCGACCACGTGGAACTGTCTTGGTGGGTCAACGGCAAAGAGGTGCACAGCGGCGTGTCC
ACCGATCCCCAGCCTCTGAAAGAACAGCCCGCCCTGAACGACAGCCGGTACTGCCTGAGCAGCAGACTG
AGAGTGTCCGCCACCTTCTGGCAGAACCCCAGAAACCACTTCAGATGCCAGGTGCAGTTTTACGGCCTG
AGCGAGAACGACGAGTGGACCCAGGACAGAGCCAAGCCCGTGACACAGATCGTGTCTGCCGAAGCTTGG
GGGCGCGCCGATTGTGGCTTTACCAGCGAGAGCTACCAGCAGGGCGTGCTGAGCGCCACCATCCTGT
ACGAGATCCTGCTGGGAAAGGCCACACTGTACGCCGTGCTGGTGTCTGCCCTGGTGCTGATGGCCATGG
TCAAGCGGAAGGACAGCCGGGGCTAA
Synthetic PolyA sequence
SEQ ID NO: 46
AATAAAATATCTTTATTTTCATTACATCTGTGTGTTGGTTTTTTGTGTG
EF1A promoter
SEQ ID NO: 47
GCTCCGGTGCCCGTCAGTGGGCAGAGCGCACATCGCCCACAGTCCCCGAGAAGTTGGGGGGAGGGGTCG
GCAATTGAACCGGTGCCTAGAGAAGGTGGCGCGGGGTAAACTGGGAAAGTGATGTCGTGTACTGGCTCC
GCCTTTTTCCCGAGGGTGGGGGAGAACCGTATATAAGTGCAGTAGTCGCCGTGAACGTTCTTTTTC
GCAACGGGTTTGCCGCCAGAACACAG
LOXP
SEQ ID NO: 48
ATAACTTCGTATAATGTATGCTATACGAAGTTAT
Puromycin resistance gene
SEQ ID NO: 49
ATGACC GAGTACAAGC CCACCGTGAG GCTGGCTACC AGAGACGACG TGCCCAGAGC
CGTGAGAACC CTGGCTGCCG CCTTCGCTGA CTACCCTGCC ACCAGGCACA CCGTGGATCC
CGACAGGCAC ATCGAGAGGG TGACCGAGCT GCAGGAGCTG TTCCTGACCA GGGTGGGCCT
GGACATCGGC AAGGTGTGGG TGGCTGACGA TGGAGCCGCT GTGGCCGTGT GGACAACCCC
CGAGAGCGTG GAAGCTGGAG CCGTGTTCGC CGAGATCGGC CCTAGAATGG CCGAGCTGTC
CGGCAGCAGG CTGGCTGCTC AGCAGCAGAT GGAGGGCCTG CTGGCCCCTC ACAGACCTAA
GGAGCCCGCC TGGTTCCTGG CCACCGTGGG AGTGAGCCCT GACCACCAGG GCAAGGGACT
GGGCTCCGCT GTGGTGCTGC CTGGAGTGGA AGCCGCTGAG AGAGCCGGAG TGCCCGCCTT
TCTGGAAACC TCCGCCCCCA GGAACCTGCC CTTCTACGAG AGGCTGGGCT TCACCGTGAC
CGCCGATGTG GAAGTGCCTG AGGGCCCCAG GACCTGGTGC ATGACCAGGA AGCCCGGCGC C
Primer 1824
(SEQ ID NO: 50)
TAAGTTGGCTGTAAACAAAGTTGAA
Primer 1825
(SEQ ID NO: 51)
GCACGTCGTCTCGGTAGC
Primer 2294
(SEQ ID NO: 52)
TCTACGAGAGGCCTGGGCTTC
Primer 2295
(SEQ ID NO: 53)
GTGGGGTGGCAGGTATTAGG
Primer ITR_F
(SEQ ID NO: 54)
GGAACCCCTAGTGATGGAGTT
Primer ITR_R
(SEQ ID NO: 55)
CGGCCTCAGTGAGCGA
Sequence of P2A puro (see FIG. 2)
P2a sequence not underlined, Puromycin underlined
SEQ ID NO: 56
GGAAGCGGAGCTACTAACTTCAGCCTGCTGAAGCAGGCTGGAGACGTGGAGGAGAACCCTGGACCTATGACCGA
GTACAAGCCCACCGTGAGGCTGGCTACCAGAGACGACGTGCCCAGAGCCGTGAGAACCCTGGCTGCCGCCTTCG
CTGACTACCCTGCCACCAGGCACACCGTGGATCCCGACAGGCACATCGAGAGGGTGACCGAGCTGCAGGAGCTG
TTCCTGACCAGGGTGGGCCTGGACATCGGCAAGGTGTGGGTGGCTGACGATGGAGCCGCTGTGGCCGTGTGGAC
AACCCCCGAGAGCGTGGAAGCTGGAGCCGTGTTCGCCGAGATCGGCCCTAGAATGGCCGAGCTGTCCGGCAGCA
GGCTGGCTGCTCAGCAGCAGATGGAGGGCCTGCTGGCCCCTCACAGACCTAAGGAGCCCGCCTGGTTCCTGGCC
ACCGTGGGAGTGAGCCCTGACCACCAGGGCAAGGGACTGGGCTCCGCTGTGGTGCTGCCTGGAGTGGAAGCCGC
TGAGAGAGCCGGAGTGCCCGCCTTTCTGGAAACCTCCGCCCCCAGGAACCTGCCCTTCTACGAGAGGCTGGGCT
TCACCGTGACCGCCGATGTGGAAGTGCCTGAGGGCCCCAGGACCTGGTGCATGACCAGGAAGCCCGGCGCC
Sequence of P2aPuroFuT2AA2M4 transgene (see FIG. 2)
P2a sequence not underlined, Puromycin underlined, T2a sequence dashed underlined,
A2M4 α chain dotted underlined, A2M4 β chain double underlined
SEQ ID NO: 57
GGAAGCGGAGCTACTAACTTCAGCCTGCTGAAGCAGGCTGGAGACGTGGAGGAGAACCCTGGACCTATGACCGA
GTACAAGCCCACCGTGAGGCTGGCTACCAGAGACGACGTGCCCAGAGCCGTGAGAACCCTGGCTGCCGCCTTCG
CTGACTACCCTGCCACCAGGCACACCGTGGATCCCGACAGGCACATCGAGAGGGTGACCGAGCTGCAGGAGCTG
TTCCTGACCAGGGTGGGCCTGGACATCGGCAAGGTGTGGGTGGCTGACGATGGAGCCGCTGTGGCCGTGTGGAC
AACCCCCGAGAGCGTGGAAGCTGGAGCCGTGTTCGCCGAGATCGGCCCTAGAATGGCCGAGCTGTCCGGCAGCA
GGCTGGCTGCTCAGCAGCAGATGGAGGGCCTGCTGGCCCCTCACAGACCTAAGGAGCCCGCCTGGTTCCTGGCC
ACCGTGGGAGTGAGCCCTGACCACCAGGGCAAGGGACTGGGCTCCGCTGTGGTGCTGCCTGGAGTGGAAGCCGC
TGAGAGAGCCGGAGTGCCCGCCTTTCTGGAAACCTCCGCCCCCAGGAACCTGCCCTTCTACGAGAGGCTGGGCT

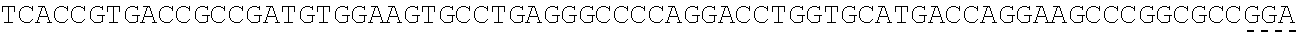

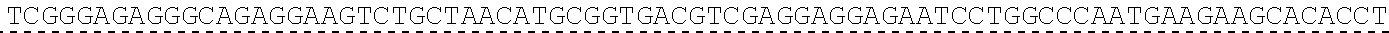

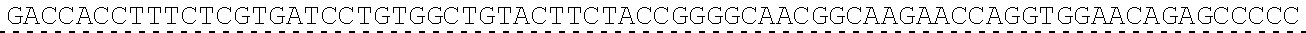

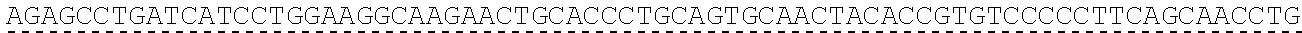

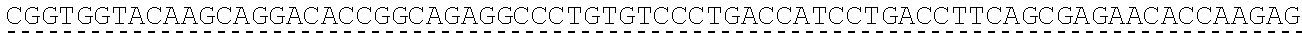

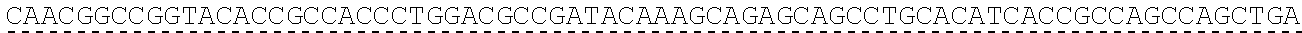

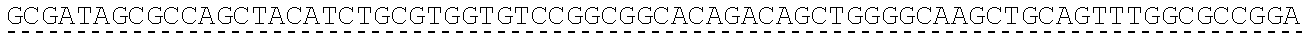

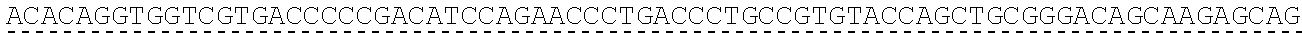

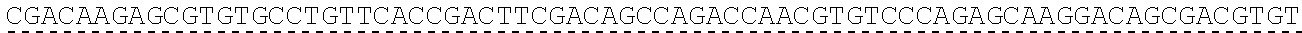

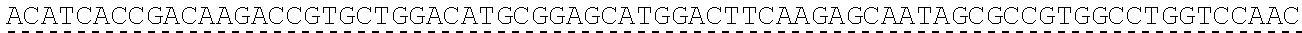

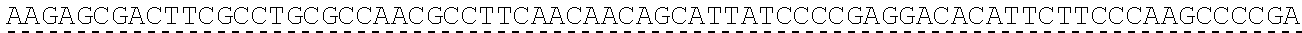

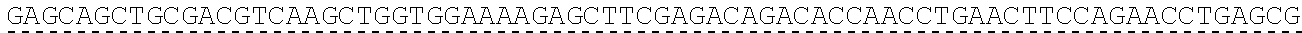

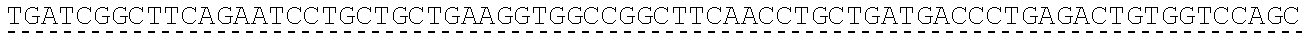

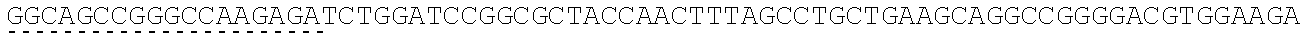

AAACCCTGGCCCTAGGATGGCCAGCCTGCTGTTCTTCTGCGGCGCCTTCTACCTGCTGGGCACCGGCTCTATGG
ATGCCGACGTGACCCAGACCCCCCGGAACAGAATCACCAAGACCGGCAAGCGGATCATGCTGGAATGCTCCCAG
ACCAAGGGCCACGACCGGATGTACTGGTACAGACAGGACCCTGGCCTGGGCCTGCGGCTGATCTACTACAGCTT
CGACGTGAAGGACATCAACAAGGGCGAGATCAGCGACGGCTACAGCGTGTCCAGACAGGCTCAGGCCAAGTTCA
GCCTGTCCCTGGAAAGCGCCATCCCCAACCAGACCGCCCTGTACTTTTGTGCCACAAGCGGCCAGGGCGCCTAC
GAGGAGCAGTTCTTTGGCCCTGGCACCCGGCTGACAGTGCTGGAAGATCTGAAGAACGTGTTCCCCCCAGAGGT
GGCCGTGTTCGAGCCTTCTGAGGCCGAAATCAGCCACACCCAGAAAGCCACACTCGTGTGTCTGGCCACCGGCT
TCTACCCCGACCACGTGGAACTGTCTTGGTGGGTCAACGGCAAAGAGGTGCACAGCGGCGTGTCCACCGATCCC
CAGCCTCTGAAAGAACAGCCCGCCCTGAACGACAGCCGGTACTGCCTGAGCAGCAGACTGAGAGTGTCCGCCAC
CTTCTGGCAGAACCCCAGAAACCACTTCAGATGCCAGGTGCAGTTTTACGGCCTGAGCGAGAACGACGAGTGGA
CCCAGGACAGAGCCAAGCCCGTGACACAGATCGTGTCTGCCGAAGCTTGGGGGCGCGCCGATTGTGGCTTTACC
AGCGAGAGCTACCAGCAGGGCGTGCTGAGCGCCACCATCCTGTACGAGATCCTGCTGGGAAAGGCCACACTGTA
CGCCGTGCTGGTGTCTGCCCTGGTGCTGATGGCCATGGTCAAGCGGAAGGACAGCCGGGGC

1. A method of producing a population of T cells comprising;
(i) introducing a heterologous nucleic acid encoding T-cell receptor at or into a locus in the cell genome of a population of iPSCs, and
(iii) differentiating the population of iPSCs into T cells;
wherein the locus is the eukaryotic translation elongation factor 1 alpha 1 (EEF1A1) gene.

2. A method according to claim 1 wherein the heterologous nucleic acid encoding the TCR is comprised in an expression vector.

3. A method according to claim 2 wherein the expression vector is a lentiviral vector or adeno-associated viral (AAV) vector.

4. A method according to claim 3 wherein the lentiviral vector is a VSVg-pseudotyped viral vector.

5. A method according to any one of the preceding claims wherein the T cells express or present the TCR encoded by the heterologous nucleic acid, preferably expressed or presented at the cell surface.

6. A method according to any one of the preceding claims wherein the heterologous nucleic acid is integrated at or into one or both alleles of the locus in the cell genome following said introduction.

7. A method according to any one of the preceding claims wherein the heterologous nucleic acid is integrated within an intron or exon of the EEF1A1 gene, optionally a 3′ exon, following said introduction.

8. A method according to claim 7 wherein the heterologous nucleic acid sequence encoding a heterologous TCR is integrated within the 3′ exon before the TAG stop sequence of the EEF1A1 gene.

9. A method according to any one of the preceding claims wherein the integration of the heterologous nucleic acid sequence encoding a heterologous TCR is non-disruptive to the production of endogenous EEF1A1 following said introduction.

10. A method according to any one of the preceding claims wherein the introduction of the heterologous nucleic acid produces a fusion sequence between the nucleic acid encoding the heterologous TCR and the nucleic acid encoding EEF1A1, preferably a fusion gene or sequence or multicistronic fusion gene or sequence between the nucleic acid encoding the heterologous TCR and the nucleic acid encoding the endogenous EEF1A1.

11. A method according to claim 10 wherein the nucleic acid encoding the heterologous TCR is connected to the nucleic acid encoding EEF1A1 by a nucleic acid sequence encoding a peptide comprising an enzymatic cleavage site and/or a nucleic acid sequence which mediates ribosome-skipping.

12. A method according to any one of the preceding claims wherein the TCR is an affinity enhanced TCR.

13. A method according to any one of the preceding claims wherein the TCR is a αβ TCR or γδ TCR.

14. A method according to any one of the preceding claims wherein the nucleic acid encoding the heterologous TCR comprises a coding sequence of a TCRα and TCRβ chain, optionally with an intervening nucleic acid sequence encoding a peptide comprising an enzymatic cleavage site and/or nucleic acid sequence which mediates ribosome-skipping.

15. A method according to claim 14 wherein the nucleic acid sequence which mediates ribosome-skipping is a T2A or P2A skip sequence.

16. A method according to claim 14 wherein the nucleic acid sequence encoding a peptide comprising an enzymatic cleavage site encodes a furin cleavage site, preferably RAKR.

17. A method according to any one of the preceding claims wherein the TCR binds specifically to an MHC displaying a peptide fragment of a target antigen expressed by cells.

18. A method according to claim 17 wherein the TCR binds specifically to an MHC displaying a peptide fragment of a tumour antigen expressed by the cancer cells.

19. A method according to claim 18 wherein the tumour antigen is alpha-fetoprotein (AFP), NY-ESO1, MAGE-A10 or MAGE-A4.

20. A method according to any one of the preceding claims wherein the population of induced pluripotent stem cells (iPSCs) is differentiated into T cells by method comprising;
differentiating the population of iPSCs into haemogenic lineage cells and
differentiating the haemogenic lineage cells into T cells.

21. A method according to any one of the preceding claims wherein the population of induced pluripotent stem cells (iPSCs) is differentiated into T cells by method comprising;
(i) differentiating a population of induced pluripotent stem cells (iPSCs) into mesoderm cells,
(ii) differentiating the mesoderm cells (MCs) to produce a population of haemogenic endothelial cells (HECs),
(iii) differentiating the HECs into a population of haematopoietic progenitor cells (HPCs),
(iv) differentiating the population of HPCs into progenitor T cells, and
(v) maturing the progenitor T cells to produce a population of double positive CD4+ CD8+ T cells.

22. A method according to claim 21 further comprising;
(vi) activating and expanding the T cells to produce a population of single positive CD8+ T cells or a population of single positive CD4+ T cells.

23. A method according to any one of claims 21 to 22 wherein the iPSCs are differentiated into mesoderm cells by culturing the population of iPSCs under suitable conditions to promote mesodermal differentiation.

24. A method according to claims 21 to 23 wherein the iPSCs are cultured sequentially in first, second and third mesoderm induction media to induce differentiation into mesoderm cells.

25. A method according to claim 24 wherein the first mesoderm induction medium stimulates SMAD2 and SMAD3 mediated signaling pathways.

26. A method according to claim 24 or 25 wherein the first mesoderm induction medium comprises activin.

27. A method according to any one of claims 24 to 26 wherein the first mesoderm induction medium consists of a chemically defined nutrient medium supplemented with one or more differentiation factors, wherein the one or more differentiation factors consist of activin.

28. A method according to any one of claims 24 to 27 wherein the second mesoderm induction medium (i) stimulates SMAD1, SMAD2, SMAD3, SMAD5 and SMAD9 mediated signaling pathways and (ii) has fibroblast growth factor (FGF) activity.

29. A method according to any one of claims 24 to 28 wherein the second mesoderm induction medium comprises activin, BMP, and FGF.

30. A method according to any one of claims 24 to 29 wherein the second mesoderm induction medium consists of a chemically defined nutrient medium supplemented with one or more differentiation factors, wherein the one or more differentiation factors consist of activin, BMP, and FGF.

31. A method according to any one of claims 24 to 30 wherein the third mesoderm induction medium (i) stimulates SMAD1, SMAD2, SMAD3, SMAD5 and SMAD9 mediated signaling pathways (ii) has fibroblast growth factor (FGF) activity and (iii) inhibits glycogen synthase kinase 3β.

32. A method according to any one of claims 24 to 31 wherein the third mesoderm induction medium comprises activin, BMP, FGF, and a GSK3 inhibitor.

33. A method according to any one of claims 24 to 32 wherein the third mesoderm induction medium consists of a chemically defined nutrient medium supplemented with one or more differentiation factors, wherein the one or more differentiation factors consist of activin, BMP, FGF, and a GSK3 inhibitor.

34. A method according to any one of claims 21 to 33 wherein the mesoderm cells display one or more of Brachyury+Goosecoid+MixI1+KDR+FoxA2+GATA6+ and PDGFαR+.

35. A method according to any one of claims 21 to 34 wherein the mesoderm cells are differentiated into HECs by culturing the population of mesoderm cells under suitable conditions to promote haemogenic endothelial (HE) differentiation.

36. A method according to any one of claims 21 to 35 wherein the mesoderm cells are cultured in an HE induction medium to induce differentiation into HECs.

37. A method according to claim 36 wherein the HE induction medium (i) stimulates KIT (KIT proto-oncogene, receptor tyrosine kinase) mediated signaling pathways and (ii) stimulates VEGFR mediated signaling pathways.

38. A method according to claim 36 or claim 37 wherein the HE induction medium comprises SCF and VEGF.

39. A method according to any one of claims 36 to 38 wherein the HE induction medium consists of a chemically defined nutrient medium supplemented with one or more differentiation factors, wherein the one or more differentiation factors consist of SCF and VEGF.

40. A method according to any one of claims 21 to 39 wherein the HPCs are differentiated into progenitor T cells under suitable conditions to promote lymphoid differentiation.

41. A method according to claim 40 wherein the HPCs are differentiated by a method comprising culturing the population of HPCs in a lymphoid expansion medium to produce the progenitor T cells.

42. A method according to claim 40 or 41 wherein the progenitor T cells have a CD5+, CD7+ phenotype.

43. A method according to any one of claims 40 to 42 wherein the progenitor T cells are matured into T cells under suitable conditions to promote T cell maturation.

44. A method according to claim 43 wherein the progenitor T cells are matured by a method comprising culturing the population of progenitor T cells in a T cell maturation medium to produce the double positive CD4⁺CD8⁺ T cells.

45. A method according to any one of claims 21 to 44 further comprising isolating or purifying the double positive CD4⁺CD8⁺ T cells and/or single positive CD4⁺ T cells or single positive CD8⁺ T cells.

46. A population of T cells comprising at least one heterologous nucleic acid encoding a T-cell receptor at or into a locus in the genome of the T cells in the population, wherein the locus is the eukaryotic translation elongation factor 1 alpha 1 (EEF1A1) gene.

47. A population of T cells produced by a method according to any one of claims 1 to 45.

48. A population according to claim 47 wherein the T cells express a heterologous T-cell receptor.

49. A population according to any one of claims 46 to 48 wherein the T cells are RAG inactivated and do not express an endogenous TCR.

50. A population of iPSCs comprising at least one heterologous nucleic acid encoding a T-cell receptor at or into a locus in the cell genome, wherein the locus is the eukaryotic translation elongation factor 1 alpha 1 (EEF1A1) gene.

51. A pharmaceutical composition comprising a population of T cells according to any one of claims 46 to 50 and a pharmaceutically acceptable excipient.

52. A population of T cells according to any one of claims 46 to 50 for use in a method of treatment.

53. A population of T cells according to any one of claims 46 to 50 for use in a method of treatment of cancer.

54. A method of treatment of cancer comprising administering a population of T cells according to any one of claims 46 to 50 to an individual in need thereof.
